SEQUENCE LISTING (20 sequences)

SEQ ID NO: 1 - protein, 12 aa, Artificial Sequence
  Synthetic construct
GGGCYFQNCPKG

SEQ ID NO: 2 - protein, 9 aa, Artificial Sequence
  Synthetic construct
CYFQNCPRG

SEQ ID NO: 3 - protein, 6 aa, Artificial Sequence
  Synthetic construct
PCAIWF

SEQ ID NO: 4 - protein, 8 aa, Artificial Sequence
  Synthetic construct
  misc_feature (3)..(3): allo-Isoleucine
GVXSQIRP

SEQ ID NO: 5 - protein, 27 aa, Artificial sequence
  Synthetic construct
SHCHWNDLAVIPAGVVHNWDFEPRKVS

SEQ ID NO: 6 - protein, 27 aa, Artificial Sequence
  Synthetic construct
GRGHLLGRLAAIVGKQVLLGRKVVVVR

SEQ ID NO: 7 - protein, 26 aa, Artificial Sequence
  Synthetic construct
SKPSFQEFVDWENVSPELNSTDQPFL

SEQ ID NO: 8 - protein, 27 aa, Artificial Sequence
  Synthetic construct
REGVELCPGNKYEMRRHGTTHSLVIHD

SEQ ID NO: 9 - protein, 15 aa, Artificial Sequence
  Synthetic construct
VDWENVSPELNSTDQ

SEQ ID NO: 10 - protein, 9 aa, Artificial Sequence
  Synthetic construct
KVPRNQDWL

SEQ ID NO: 11 - protein, 25 aa, Artificial Sequence
  Synthetic construct
CKGKGAKCSRLMYDCCTGSCRSGKC

SEQ ID NO: 12 - protein, 45 aa, Artificial Sequence
  Synthetic construct
ADNKCNSLRREIACGQCRDKVKTDGYFYECCTSDSTFKKCQDLLH

SEQ ID NO: 13 - protein, 36 aa, Artificial Sequence
  Synthetic construct
EESMLLSCPDLSCPTGYTCDVLTKKCKRLSDELWDH

SEQ ID NO: 14 - protein, 13 aa, Artificial Sequence
  Synthetic construct
GCCSDPRCRYRCR

SEQ ID NO: 15 - protein, 9 aa, Artificial Sequence
  Synthetic construct
PVNFKFLSH

SEQ ID NO: 16 - protein, 10 aa, Artificial Sequence
  Synthetic construct
  misc_feature (6)..(7): Hydroxyproline
  misc_feature (9)..(9): 3,4-dihydroxyphenylalanine (DOPA)
AKPSYXXTXK

SEQ ID NO: 17 - protein, 11 aa, Artificial Sequence
  Synthetic construct
LAGGWGERDGS

SEQ ID NO: 18 - protein, 6 aa, Artificial Sequence
  Synthetic construct
PSAIWF

SEQ ID NO: 19 - protein, 15 aa, Artificial sequence
  Synthetic construct
GPLGLAGGWGERDGS

SEQ ID NO: 20 - protein, 8 aa, Artificial Sequence
  Synthetic construct
SIINFEKL

(19) United States
(12) Patent Application Publication
(10) Pub. No.: US 2022/0280625 A1
(43) Pub. Date: Sep. 8, 2022

(54) BRUSH POLYMERS FOR THERAPEUTIC APPLICATIONS

(71) Applicant: Northwestern University, Evanston, IL (US)

(72) Inventors: Nathan C. GIANNESCHI, Evanston, IL (US); Jeremy A. LAVINE, Evanston, IL (US); Bin ZHANG, Evanston, IL (US); Or BERGER, Evanston, IL (US); Wonmin CHOI, Evanston, IL (US); Max Mu WANG, Evanston, IL (US); Claudia BATTISTELLA, Evanston, IL (US); Hao SUN, Evanston, IL (US)

(73) Assignee: Northwestern University, Evanston, IL (US)

(21) Appl. No.: 17/634,497

(22) PCT Filed: Aug. 11, 2020
(86) PCT No.: PCT/US2020/045729

§ 371 Date: Feb. 10, 2022

Related U.S. Application Data
(60) Provisional application No. 62/885,655, filed on Aug. 12, 2019.
(60) Provisional application No. 63/025,388, filed on May 15, 2020.

(51) Int. Cl. A61K 39/00; A61K 38/095; A61K 47/68; A61K 47/64; A61K 47/58; A61P 9/12; A61P 35/00

(52) U.S. Cl. CPC A61K 39/001192; A61K 38/095; A61K 47/6845; A61K 47/64; A61K 47/58; A61P 9/12; A61P 35/00; A61K 2039/6031; A61K 2039/6093; A61K 2039/627; A61K 2039/6056

(57) ABSTRACT

In an aspect, the invention provides therapeutic agents comprising brush polymers that address challenges associated with conventional administration of free therapeutic peptides. In an embodiment, for example, the invention provides brush polymers incorporating one or more therapeutic peptides as side chain moieties. Therapeutic agents of the invention comprising brush polymers include high-density brush polymers including cross-linked brush polymers and brush block copolymers. In an embodiment, brush polymers of the invention exhibit proteolysis-resistant characteristics and maintain their biological function during formulation and administration. The invention also includes methods of making and using therapeutic agents comprising brush polymers.

### CROSS-REFERENCE TO RELATED APPLICATIONS

This application claims the benefit of and priority to U.S. Provisional Patent Application No. 62/885,655 filed Aug. 12, 2019, and U.S. Provisional Patent Application No. 63/025,388 filed May 15, 2020, each of which is hereby incorporated by reference in its entirety.

### STATEMENT REGARDING FEDERALLY SPONSORED RESEARCH OR DEVELOPMENT

This invention was made with government support under Award Number NSF 1710105 awarded by the National Science Foundation. The government has certain rights in the invention.

### REFERENCE TO A SEQUENCE LISTING

A sequence listing containing SEQ ID NOs.: 1-20, created Aug. 11, 2020, 6 kB, is provided herewith in a computer-readable nucleotide/amino acid .txt file and is specifically incorporated by reference.

### BACKGROUND OF INVENTION

The use of protein and peptide therapeutics continues to increase dramatically for diverse clinical applications. However, inefficiencies in cellular uptake and rapid digestion by proteases are two problems that have limited the clinical adoption of peptide-based therapeutics. Accordingly, many peptide therapeutics are incompatible with systemic administration and, therefore, must be administered by injection at the site of action due to poor in vivo stability. This can result in poor patient compliance and, as such, many peptide therapies only are used clinically as salvage treatments.

Several approaches for producing peptides protected from proteolysis involve chemical modification of the amino acid sequence, which generally necessitates multiple rounds of structure-function studies to confirm that the activity of the peptide is not altered. Other approaches not using chemical modification of the amino acid sequence involve conjugation of the peptide to a pre-formed higher molecular weight structure, such as a polymer or nanomaterial. The downside of these approaches includes requiring additional conjugation and purification steps, as well as the formation of, and release from, the high molecular weight carrier.

Despite these challenges, there remaining significant interest in developing improved delivery systems to enhance clinical applicability and overall efficacy for therapies involving therapeutic peptides.

US Patent Publication US 2018/0042843, the disclosure of which is incorporated herein by reference, for example, discloses drug delivery vehicles involving cell penetrating high-density brush polymers including a therapeutic peptide component.

US Patent Publication US 2018/0303945, for example, describes delivery and formulation technologies to improve therapeutic properties and deliver sustained activity. According to US20180303945, there continues to be a need for new drug delivery systems suitable for the sustained release of biologically active moieties in therapeutic applications, particularly for conformationally sensitive drugs such as proteins, peptides, or antibodies rendered dysfunctional during encapsulation or subsequent storage of the encapsulated drug.

PCT International Publication WO2018/156617, for example, describes polymers associated with biotherapeutic peptides to improve efficacy of oligopeptide antigens. WO2018/156617 further describes challenges with the clinical translation of prior approaches, including manufacturing challenges, reliability of synthesizing platforms, and limited in vivo efficacy.

US Patent Publication US 20190070251, for example, discloses conjugates made from therapeutic peptide moieties covalently attached to water-soluble polymers. According to US20190070251, peptides often have unsatisfactory properties due to short in vivo half-life.

Despite the current level of understanding, there remains a need for delivery systems and methods for therapeutic peptides providing improved pharmacokinetic properties, administration routes and overall efficacies.

### SUMMARY OF THE INVENTION

In an aspect, the present compositions include brush polymer therapeutic agents comprising therapeutic peptides, including drugs and prodrugs thereof, which address challenges associated with conventional administration of a diverse class of therapeutic peptides.

In an embodiment, for example, the invention provides brush polymers incorporating one or more therapeutic peptides as side chain moieties. Therapeutic agents of the invention comprising brush polymers of some embodiments are characterized by high brush densities, including optionally cross-linked brush polymers and/or brush block copolymers. Therapeutic agents of the invention comprising brush polymers include brush polymers having polymer side chains characterized by one or more degradable linker, such as an in vivo degradable linker or triggerable linker.

In an embodiment, brush polymers of the invention exhibit proteolysis-resistant characteristics and maintain their biological function during formulation and in vivo administration to a subject. In some embodiments, conjugation of the therapeutic peptide to the brush polymer backbone renders it more resistant to in vivo degradation by proteolytic enzymes as compared to a free therapeutic peptide. Moreover, the higher molecular weight of the brush block polymer, relative to its free therapeutic peptide analogue, confers longer circulation time than the free therapeutic peptide. As a result, the therapeutic polymers can be administered less frequently and in smaller doses than the free peptide therapeutics used in the clinic. Further, the enhanced stability and resistance to degradation of the present brush polymer therapeutic agents allows for more versatility with respect to administration route and conditions, including in injection at the site of action and systemic administration.

Brush block polymers of the invention may comprises a range of therapeutic peptides including a terlipressin peptide, an anti-VEGF peptide, an ABT-898 peptide, a neoantigen-Melanoma peptide, an antigen-M30 peptide, an antigen-gp100 Melanoma peptide or a derivative, analogue, variant, isomer, or a fragment of terlipressin peptide, an anti-VEGF peptide, an ABT-898 peptide, a neoantigen-Melanoma peptide, an antigen-M30 peptide, an antigen-gp100 Melanoma peptide or a derivative, analogue, variant, isomer or fragment of any of these peptides.

The invention also includes methods of using therapeutic agents comprising brush polymers for a range of clinical applications including, by way of example, for treatment or management of conditions including hepatorenal syndrome, low blood pressure, bleeding esophageal varices, septic shock, paracentesis-induced circulatory dysfunction, age related macular degeneration, and cancer.

The invention also includes methods of making therapeutic agents comprising brush polymers, for example, via “grafting from” methods, “grafting onto” methods and “grafting through” methods. In some methods, a ring opening metathesis polymerization (ROMP) synthetic approach is used to make therapeutic agents comprising brush polymers, for example, having high graft densities and low polydispersity. The present methods of making therapeutic agents comprising brush polymers include other non-ROMP synthetic pathways such as, by way of example, reversible addition fragmentation chain transfer (RAFT) polymerization, stable free radical mediated polymerization and atom transfer radical polymerization (ATRP).

In an embodiment, the invention provides a polymer comprising a polymer comprising a first polymer block at least 2 first repeating units and optionally 2-30 or 5-30 first repeating units; wherein each of the first repeating units of the first polymer block comprises a first polymer backbone group directly or indirectly covalently linked to a first polymer side chain group comprising a therapeutic peptide.

In an aspect, a polymer is provided, the polymer is characterized by the formula (FX2a), (FX2b), or (FX2c):

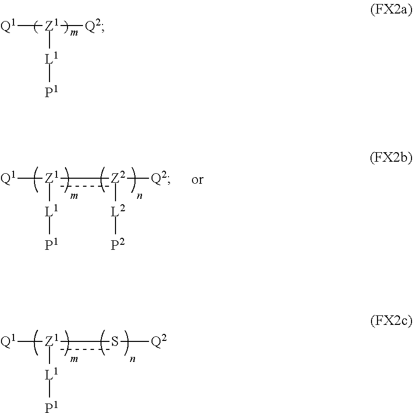

wherein each Z¹ is independently a first polymer backbone group and each Z² is independently a second polymer backbone group; each S is independently a repeating unit having a composition different from the first repeating unit; Q¹ is a first backbone terminating group and Q² is a second backbone terminating group; each Lis independently a first linking group, each L² is independently a second linking group; each P¹ is the polymer side chain comprising the peptide; wherein each P² is a polymer side chain having a composition different from that of P¹; each m is independently an integer selected from the range of 2 to 100; each n is independently an integer selected from the range of 0 to 100; and each h is independently an integer selected from the range of 0 to 100, provided that each of the first polymer backbone group and/or the second polymer backbone group is a polymerized norbornene dicarboxyimide monomer, and wherein the polymer fulfills at least one (i.e., (i), (ii), and/or (iii) of the following properties:
(i) the polymer has a degree of polymerization of 5 to 100,
(ii) the peptide comprises 5 to 100 amino acids, and
(iii) the polymer has a peptide density of greater than 50%, as defined by the following formula:

$$\frac{{P}^{1}}{{P}^{1} + {P}^{2} + S} \times 100.$$

In an aspect, a polymer is provided comprising: a first polymer block comprising at least 2 first repeating units; wherein each of the first repeating units of the first polymer block comprises a first polymer backbone group directly or indirectly covalently linked to a first polymer side chain group comprising a peptide; wherein the peptide is a terlipressin peptide, or a derivative, analogue, variant, isomer or fragment of an terlipressin peptide; and wherein the polymer exhibits efficacy for treatment or management of a condition selected from the group consisting of hepatorenal syndrome, low blood pressure, bleeding esophageal varices, septic shock and paracentesis-induced circulatory dysfunction. In an embodiment of this aspect, the peptide comprises a sequence having 80% or greater sequence homology, optionally 90% or greater, of SEQ ID NO: 1 (GGGCYFQNCPKG) or optionally a sequence having 80% or greater sequence homology, optionally 90% or greater with the full length of SEQ ID NO: 1 (GGGCYFQNCPKG). In an embodiment of this aspect, the peptide comprises the amino acid sequence of SEQ ID NO: 1 (GGGCYFQNCPKG).

In an aspect, a polymer is provided comprising: a first polymer block comprising at least 2 first repeating units; wherein each of the first repeating units of the first polymer block comprises a first polymer backbone group directly or indirectly covalently linked to a first polymer side chain group comprising a peptide; wherein the peptide is an anti-VEGF peptide, an ABT-898 peptide or a derivative, analogue, variant, isomer or fragment of an anti-VEGF peptide or an ABT-898 peptide; and wherein the polymer exhibits efficacy for treatment or management of age related macular degeneration. In an embodiment of this aspect, the peptide comprises a sequence having 80%, optionally 90% or greater, sequence homology of SEQ ID NO:3 (PCAIWF) or SEQ ID NO: 4 (GVi(allo)SQIRP), optionally a sequence having 80% or greater sequence homology, optionally 90% or greater with the full length of SEQ ID NO: 3 (PCAIWF) or SEQ ID NO: 4 (GVi(allo)SQIRP). In an embodiment of this aspect, the peptide comprises the amino acid sequence of SEQ ID NO: 3 (PCAIWF) or SEQ ID NO: 4 (GVi(allo)SQIRP).

In an aspect, a polymer is provided comprising: a first polymer block comprising at least 2 first repeating units; wherein each of the first repeating units of the first polymer block comprises a first polymer backbone group directly or indirectly covalently linked to a first polymer side chain group comprising a peptide; wherein the peptide is a neoantigen—melanoma peptide or a derivative, analogue, variant, isomer or fragment thereof; and wherein the polymer exhibits efficacy for treatment or management of cancer. In an embodiment of this aspect, the peptide comprises a sequence having 80%, optionally 90% or greater, sequence homology of SEQ ID NO:5 (SHCHWNDLAVIPAGVVHNWDFEPRKVS), SEQ ID NO: 6. (GRGHLLGRLAAIVGKQVLLGRKVVVVR), SEQ ID NO: 7. (SKPSFQEFVDWENVSPELNSTDQPFL), or SEQ ID NO: 8 (REGVELCPGNKYEMRRHGTTHSLVIHD), optionally a sequence having 80% or greater sequence homology, optionally 90% or greater with the full length of SEQ ID NO: 5 (SHCHWNDLAVIPAGVVHNWDFEPRKVS), SEQ ID NO: 6. (GRGHLLGRLAAIVGKQVLLGRKVVVVR), SEQ ID NO: 7. (SKPSFQEFVDWENVSPELNSTDQPFL), or SEQ ID NO: 8 (REGVELCPGNKYEMRRHGTTHSLVIHD). In an embodiment of this aspect, the peptide comprises the amino acid sequence of SEQ ID NO: 5 (SHCHWNDLAVIPAGVVHNWDFEPRKVS), SEQ ID NO: 6. (GRGHLLGRLAAIVGKQVLLGRKVVVVR), SEQ ID NO: 7. (SKPSFQEFVDWENVSPELNSTDQPFL), or SEQ ID NO: 8 (REGVELCPGNKYEMRRHGTTHSLVIHD).

In an aspect, a polymer is provided comprising: a first polymer block comprising at least 2 first repeating units; wherein each of the first repeating units of the first polymer block comprises a first polymer backbone group directly or indirectly covalently linked to a first polymer side chain group comprising a peptide; wherein the peptide is an antigen-M30 peptide, an antigen-gp100 melanoma peptide or a derivative, analogue, variant, isomer or fragment of an antigen-M30 peptide or an antigen-gp100 melanoma peptide; and wherein the polymer exhibits efficacy for treatment or management of cancer. In an embodiment of this aspect, the peptide comprises a sequence having 80%, optionally 90% or greater, sequence homology of SEQ ID NO: 9 (VDWENVSPELNSTDQ) or SEQ ID NO: 10 (KVPRNQDWL), optionally a sequence having 80% or greater sequence homology, optionally 90% or greater with the full length of SEQ ID NO: 9 (VDWENVSPELNSTDQ) or SEQ ID NO: 10 (KVPRNQDWL). In an embodiment of this aspect, the peptide comprises the amino acid sequence of SEQ ID NO: 9 (VDWENVSPELNSTDQ) or SEQ ID NO: 10 (KVPRNQDWL).

In an embodiment, the polymer is a homopolymer, optionally a copolymer. In an embodiment, the polymer is a brush polymer, optionally a brush block copolymer. In an embodiment, the first polymer block of the polymer comprises at least 5 first repeating units, optionally 5-30 first repeating units. In an embodiment, the polymer is characterized by a degree of polymerization of 2 to 1000. In an embodiment, the polymer is characterized by a polydispersity index less than 1.75.

In an embodiment, the peptide is a polypeptide comprising 2 to 100 amino acid units. In an embodiment, the peptide is a branched polypeptide, a linear polypeptide or a cross-linked polypeptide. In an embodiment, the polymer is characterized by a structure wherein at least a portion of the peptide is linked to the polymer backbone group via an enzymatically degradable linker, such a matrix metalloproteinase (MMP) cleavage sequence, cathepsin B cleavage sequence, ester bond, reductive sensitive bond—disulfide bond, pH sensitive bond—imine bond or any combinations of these. In an embodiment, the polymer is characterized by a structure wherein at least a portion of the peptide side-chain is linked to the polymer backbone or consists of a degradable or triggerable linker.

In an embodiment, the polymer is characterized by the formula (FX1a), (FX1b), (FX1c), (FX1d); (FX1e); (FX1f); or (FX1g):

Q¹-T-Q² (FX1a);

Q¹-T-[S]ₕ-Q² (FX1b);

Q¹-[S]ₕ-T-Q² (FX1c);

Q¹-[S]ᵢ-T-[S]ₕ-Q² (FX1d);

Q¹-[S]ᵢ-T-[S]ₕ-T-Q² (FX1e);

Q¹-T-[S]ᵢ-T-[S]ₕ-Q² (FX1f);or

Q¹-T-[S]ᵢ-T-[S]ₕ-T-Q² (FX1g);

wherein each T is independently the first polymer block comprising the first repeating units and each S is independently an additional polymer block; Q¹ is a first polymer block terminating group; Q² is a second polymer block terminating group; and wherein h is zero or an integer selected over the range of 1 to 1000 and i is zero or an integer selected over the range of 1 to 1000. In an embodiment, the polymer is characterized by any of formulas (FX1a)-(FX1g), wherein each -T- is independently -[Y¹]ₘ-; wherein each Y¹ is independently the first repeating unit of the first polymer block; and each m is independently an integer selected from the range 0 to 1000, provided that at least one m is an integer selected from the range 1 to 1000.

In an embodiment, the polymer is characterized by the formula (FX2a), (FX2b), or (FX2c):

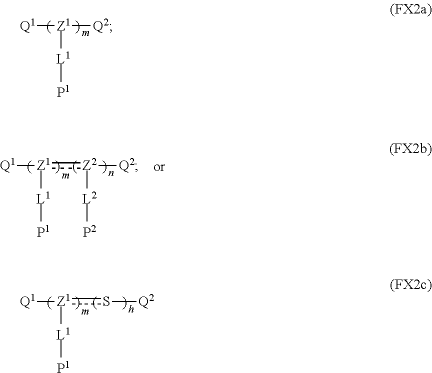

wherein each Z¹ is independently a first polymer backbone group and each Z² is independently a second polymer backbone group; wherein each S is independently a repeating unit having a composition different from the first repeating unit; the wherein Q¹ is a first backbone terminating group and Q² is a second backbone terminating group; wherein each L¹ is independently a first linking group, each L² is independently a second linking group; wherein each P¹ is the polymer side chain comprising the peptide; wherein each P² is a polymer side chain having a composition different from that of P¹; and wherein each m is independently an integer selected from the range of 2 to 1000 (e.g., 2 to 500, 2 to 250, or 2 to 100); wherein each n is each independently an integer selected from the range of 0 to 1000 (e.g., 0 to 500, 0 to 250, or 0 to 100); and wherein h are each independently an integer selected from the range of 0 to 1000 (e.g., 0 to 500, 0 to 250, or 0 to 100).

In an embodiment, the polymer is characterized by any of formulas (FX2a)-(FX2c), wherein each Z¹ connected to L¹, and P¹ or a combination thereof is independently characterized by the formula (FX3a) or (FX3b):

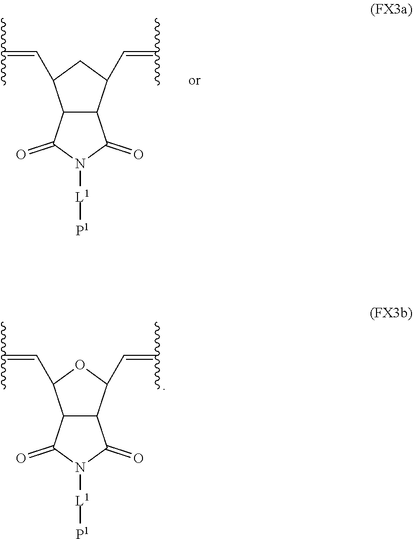

and wherein each Z² connected to L², and P² or a combination thereof is independently characterized by the formula (FX4a) or (FX4b)

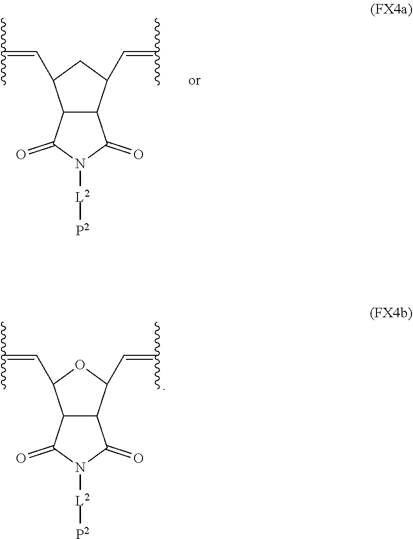

In an embodiment, the polymer is characterized by any of formulas (FX2a)-(FX2c), wherein each of Z¹ and Z² is independently a substituted or unsubstituted norbornene, oxanorbornene, olefin, cyclic olefin, cyclooctene, or cyclopentadiene. In an embodiment, the polymer is characterized by any of formulas (FX2a)-(FX2c), wherein each of Q¹ and Q² is independently selected from a hydrogen, C₁-C₃₀ alkyl, C₃-C₃₀ cycloalkyl, C₅-C₃₀ aryl, C₅-C₃₀ heteroaryl, C₁-C₃₀ acyl, C₁-C₃₀ hydroxyl, C₁-C₃₀ alkoxy, C₂-C₃₀ alkenyl, C₂-C₃₀ alkynyl, C₅-C₃₀ alkylaryl, —CO₂R³, —CONR⁴R⁵, —COR⁶, —SOR⁷, —OSR⁸, —SO₂R⁹, —OR¹⁰, —SR¹¹, —NR¹²R¹³, —NR¹⁴COR¹⁵, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R³-R¹⁵ is independently H, C₅-C₁₀ aryl or C₁-C₁₀ alkyl. In an embodiment, the polymer is characterized by any of formulas (FX2a)-(FX2c), wherein each of L¹ and L² is independently selected from a single bond, an oxygen, and groups having an alkyl group, an alkenylene group, an arylene group, an alkoxy group, an acyl group, a triazole group, a diazole group, a pyrazole group, and combinations thereof. In an embodiment, the polymer is characterized by any of formulas (FX2a)-(FX2c), wherein each of L¹ and L² is independently selected from a single bond, —O—, C₁-C₁₀ alkyl, C₂-C₁₀ alkenylene, C₃-C₁₀ arylene, alkoxy, acyl and combinations thereof.

In an embodiment, the polymer is characterized by any of formulas (FX2a)-(FX2c), wherein P¹ is a terlipressin peptide, an anti-VEGF peptide, an ABT-898 peptide, a neoantigen-Melanoma peptide, an antigen-M30 peptide, an antigen-gp100 Melanoma peptide, or a derivative, a variant, an analogue, or a fragment of terlipressin peptide, an anti-VEGF peptide, an ABT-898 peptide, a neoantigen-melanoma peptide, an antigen-M30 peptide, or an antigen-gp100 Melanoma peptide.

In an embodiment, the polymer is characterized by any of formulas (FX2a)-(FX2c), wherein P¹ is the peptide comprising a sequence having 80% or greater, optionally 90% or greater, sequence homology of SEQ ID NO: 1 (GGGCYFQNCPKG), SEQ ID NO: 3 (PCAIWF), SEQ ID NO: 4 (GVi(allo)SQIRP), SEQ ID NO: 5 (SHCHWNDLAVIPAGVVHNWDFEPRKVS), SEQ ID NO: 6. (GRGHLLGRLAAIVGKQVLLGRKVVVVR), SEQ ID NO: 7. (SKPSFQEFVDWENVSPELNSTDQPFL), or SEQ ID NO: 8 (REGVELCPGNKYEMRRHGTTHSLVIHD), SEQ ID NO: 9 (VDWENVSPELNSTDQ) or SEQ ID NO: 10 (KVPRNQDWL), SEQ ID NO: 11 (CKGKGAKCSRLMYDCCTGSCRSGKC), SEQ ID NO: 12. (ADNKCNSLRREIACGQCRDKVKTDGYFYECCTSDSTFKKCQDLLH), SEQ ID NO: 13. (EESMLLSCPDLSCPTGYTCDVLTKKCKRLSDELWDH), SEQ ID NO: 14 (GCCSDPRCRYRCR) or SEQ ID NO: 15. (PVNFKFLSH), or SEQ ID NO: 16 (AKPSY-Hyp-Hyp-T-DOPA-K), optionally having 80% or greater, optionally 90% or greater, of the full length sequence homology of SEQ ID NO: 1 (GGGCYFQNCPKG), SEQ ID NO: 3 (PCAIWF), SEQ ID NO: 4 (GVi(allo)SQIRP), SEQ ID NO: 5 (SHCHWNDLAVIPAGVVHNWDFEPRKVS), SEQ ID NO: 6. (GRGHLLGRLAAIVGKQVLLGRKVVVVR), SEQ ID NO: 7. (SKPSFQEFVDWENVSPELNSTDQPFL), or SEQ ID NO: 8 (REGVELCPGNKYEMRRHGTTHSLVIHD), SEQ ID NO: 9 (VDWENVSPELNSTDQ) or SEQ ID NO: 10 (KVPRNQDWL), SEQ ID NO: 11 (CKGKGAKCSRLMYDCCTGSCRSGKC), SEQ ID NO: 12. (ADNKCNSLRREIACGQCRDKVKTDGYFYECCTSDSTFKKCQDLLH), SEQ ID NO: 13. (EESMLLSCPDLSCPTGYTCDVLTKKCKRLSDELWDH), SEQ ID NO: 14 (GCCSDPRCRYRCR) or SEQ ID NO: 15. (PVNFKFLSH), or SEQ ID NO: 16 (AKPSY-Hyp-Hyp-T-DOPA-K).

In an embodiment, the polymer is characterized by any of formulas (FX2a)-(FX2c), wherein P¹ is the peptide comprising the amino acid sequence of SEQ ID NO: 1 (GGGCYFQNCPKG), SEQ ID NO: 3 (PCAIWF), SEQ ID NO: 4 (GVi(allo)SQIRP), SEQ ID NO: 5 (SHCHWNDLAVIPAGVVHNWDFEPRKVS), SEQ ID NO: 6. (GRGHLLGRLAAIVGKQVLLGRKVVVVR), SEQ ID NO: 7. (SKPSFQEFVDWENVSPELNSTDQPFL), or SEQ ID NO: 8 (REGVELCPGNKYEMRRHGTTHSLVIHD), SEQ ID NO: 9 (VDWENVSPELNSTDQ) or SEQ ID NO: 10 (KVPRNQDWL), SEQ ID NO: 11 (CKGKGAKCSRLMYDCCTGSCRSGKC), SEQ ID NO: 12. (ADNKCNSLRREIACGQCRDKVKTDGYFYECCTSDSTFKKCQDLLH), SEQ ID NO: 13. (EESMLLSCPDLSCPTGYTCDVLTKKCKRLSDELWDH), SEQ ID NO: 14 (GCCSDPRCRYRCR) or SEQ ID NO: 15. (PVNFKFLSH), or SEQ ID NO: 16 (AKPSY-Hyp-Hyp-T-DOPA-K).

In an aspect, provided are methods of treatment comprising administering to a subject an effective amount of any of the polymers disclosed herein.

In an aspect, provided are methods of treating or managing a condition in a subject comprising: administering to a subject an effective amount of a polymer comprising: a first polymer block comprising at least 2 first repeating units; wherein each of the first repeating units of the first polymer block comprises a first polymer backbone group directly or indirectly covalently linked to a first polymer side chain group comprising a peptide; wherein the peptide is a terlipressin peptide or a derivative, analogue, variant, isomer or fragment of an terlipressin peptide; wherein the condition is selected from the group consisting of hepatorenal syndrome, low blood pressure, bleeding esophageal varices, septic shock and paracentesis-induced circulatory dysfunction.

In an aspect, provided are methods of treating or managing a condition in a subject comprising: administering to a subject an effective amount of a polymer comprising: a first polymer block comprising at least 2 first repeating units; wherein each of the first repeating units of the first polymer block comprises a first polymer backbone group directly or indirectly covalently linked to a first polymer side chain group comprising a peptide; wherein the peptide is an anti-VEGF peptide, an ABT-898 peptide or a derivative, analogue, variant, isomer or fragment of an anti-VEGF peptide or an ABT-898 peptide; wherein the condition is age related macular degeneration.

In an aspect, provided are methods of treating or managing a condition in a subject comprising: administering to a subject an effective amount of a polymer comprising: a first polymer block comprising at least 2 first repeating units; wherein each of the first repeating units of the first polymer block comprises a first polymer backbone group directly or indirectly covalently linked to a first polymer side chain group comprising a peptide; wherein the peptide is a neoantigen-melanoma peptide or a derivative, analogue, variant, isomer or fragment thereof; wherein the condition is cancer.

In an aspect, provided are methods of treating or managing a condition in a subject comprising: administering to a subject an effective amount of a polymer comprising: a first polymer block comprising at least 2 first repeating units; wherein each of the first repeating units of the first polymer block comprises a first polymer backbone group directly or indirectly covalently linked to a first polymer side chain group comprising a peptide; wherein the peptide is an antigen-M30 peptide, an antigen-gp100 melanoma peptide or a derivative, analogue, variant, isomer or fragment of an antigen-M30 peptide or an antigen-gp100 melanoma peptide; wherein the condition is cancer.

In an embodiment, any of the present methods further comprise contacting a target tissue of the subject with the polymer or a metabolite or product thereof. In an embodiment, any of the present methods further comprise contacting a target cell of the subject with the polymer or a metabolite or product thereof. In an embodiment, any of the present methods further comprise contacting a target receptor of the subject with the polymer or a metabolite or product thereof.

Preferably in any embodiment of a polymer, a method, or a formulation disclosed herein, each of the first polymer backbone group and/or the second polymer backbone group is a polymerized norbornene dicarboxyimide monomer. Preferably in any embodiment of a polymer, a method, or a formulation disclosed herein, each of the first polymer backbone group and the second polymer backbone group is a polymerized norbornene dicarboxyimide monomer. Preferably in any embodiment of a polymer, a method, or a formulation disclosed herein, each of the first polymer backbone group is a polymerized norbornene dicarboxyimide monomer. Preferably in any embodiment of a polymer, a method, or a formulation disclosed herein, each polymer backbone group of the polymer is a polymerized norbornene dicarboxyimide monomer. Optionally in any embodiment of a polymer, a method, or a formulation disclosed herein, each of the first polymer backbone group and/or the second polymer backbone group comprises a polymerized norbornene dicarboxyimide monomer. Optionally in any embodiment of a polymer, a method, or a formulation disclosed herein, each of the first polymer backbone group and the second polymer backbone group comprises a polymerized norbornene dicarboxyimide monomer. Optionally in any embodiment of a polymer, a method, or a formulation disclosed herein, each of the first polymer backbone group comprises a polymerized norbornene dicarboxyimide monomer. Optionally in any embodiment of a polymer, a method, or a formulation disclosed herein, each polymer backbone group of the polymer comprises a polymerized norbornene dicarboxyimide monomer.

Preferably in any embodiment of a polymer, a method, or a formulation disclosed herein, the polymer is stable against enzymatic digestion. Optionally in any embodiment of fa polymer, a method, or a formulation disclosed herein, the polymer is stable against enzymatic digestion by a metalloproteinase. Optionally in any embodiment of a polymer, a method, or a formulation disclosed herein, the polymer is stable against enzymatic digestion by matrix metalloproteinases and thermolysin. Preferably in any embodiment of a polymer, a method, or a formulation disclosed herein, the polymer is stable against enzymatic digestion for at least 450 minutes. Optionally in any embodiment of a polymer, a method, or a formulation disclosed herein, the polymer is stable against enzymatic digestion by thermolysin such that less than 20% of thermolysin-cleavable sites are cleaved by thermolysin after at least 450 minutes of the polymer's exposure to thermolysin. Optionally in any embodiment of a polymer, a method, or a formulation disclosed herein, each polymer individually solvated by water when a plurality of said polymers is dispersed in water.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 depicts the primary structures of vasopressin (SEQ ID NO: 2) and terlipressin (SEQ ID NO: 1).

FIG. 2 depicts the G-protein coupled V1 receptor system on vascular smooth muscle cell.

FIG. 3 depicts the structure of a terlipressin peptide monomer.

FIG. 4 is a RP-HPLC chromatogram confirming the purity of a terlipressin peptide monomer prepared according to the Examples.

FIG. 5 is the mass spectrum (ESI-MS) of a terlipressin peptide monomer prepared according to the Examples.

FIG. 6 depicts the polymerization reaction of a terlipressin (SEQ ID NO:1) peptide monomer prepared according to the Examples and the ¹H NMR spectra for the time course experiments monitoring the polymerization reaction. The disappearance of the resonance at δ=6.50 ppm (blue arrow) corresponding to the olefin protons of the monomer and the coincident appearance of resonances at δ=5.7-6 ppm (orange arrows), which correspond to the cis and trans olefin protons of the polymer backbone.

FIG. 7 depicts the SEC-MALS chromatograms for terlipressin peptide monomers prepared according to the Examples and depicts the molecular weights, degree of polymerization, and the polydispersity index.

FIG. 8 depicts the structure of the GGG enzymatic cleavage site of a terlipressin peptide monomer.

FIG. 9A-9B depicts the result of an enzymatic cleavage reaction. Twenty microliters (20 μL) of elastase (1 mM), 20 μL of monomer (FIG. 9A) or polymer (FIG. 9B) (2 mM), 20 μL Tris buffer (250 mM Tris pH 7.2, 1500 mM NaCl, 500 mM CaCl₂)) and 140 μL of water were added to a HPLC vial and loaded in the autosampler carousel and a sample immediately taken for injection at 1 min. Subsequently, the reaction was monitored via RP-HPLC (10-80% buffer B) with an injection every 50 minutes following the initial injection.

FIG. 10 depicts the ratio amount of peptide remaining after incubation with elastase. The ratio of the AUC at each time point relative to the first time point is plotted. Approximately 50% of the peptide remained after 550 min.

FIG. 11 is structure of the enzymatic cleavage product and the mass spectrum (ESI-MS).

FIG. 12 depicts human aortic smooth muscle cells as used in the in vitro experiments described in the examples.

FIG. 13 depicts the fluorescence measurements upon treating human aortic smooth muscle cells with terlipressin and polymers as described in the Examples.

FIGS. 14A-14D Show formulas of peptide brush polymers with different polymer backbone groups. Molecular structure of (FIG. 14A) peptide norbornene imide monomer; (FIG. 14B) peptide norbornene amide monomer; (FIG. 14C) peptide brush polymer made of peptide norbornene imide monomer (representative DP=15), and (FIG. 14D) peptide brush polymer made of peptide norbornene amide monomer (representative DP=15). Amino acid sequences incorporated as peptide side chains are tunable and are dependent on the target applications.

FIG. 15. Synthesis of peptide brush polymers with different polynorbornene backbones via ROMP of peptide norbornene dicarboxyimide (PepNorIm) and peptide norbornene amide (PepNorAm), respectively. Grubbs III refers to third generation of Grubbs catalyst.

FIGS. 16A-16B. ¹H NMR spectra of (FIG. 16A) Poly(PepNorIm) and (FIG. 16B) Poly(PepNorAm). Amino acid sequence is GPLGLAGGWGERDGS.

FIGS. 17A-17B. Molecular weight information of peptide brush polymers with amino acid sequence GPLGLAGGWGERDGS. FIG. 17A. GPC traces of peptide brush polymers in organic solvent (DMF). FIG. 17B. GPC traces of peptide brush polymers in PBS buffer (0.01 M).

FIGS. 18A-18C. DLS traces of (FIG. 18A) poly(PepNorIm15) and (FIG. 18B) poly(PepNorAm15) in aqueous solution. FIG. 18C. Cryo-electron microscopy image of a representative polymer poly(PepNorAm15).

FIG. 19. Enzymatic cleavage of peptide brush polymers (Poly(PepNorIm15) in the presence of thermolysin.

FIG. 20. RP-HPLC monitoring enzymatic digestion of peptide brush polymers with the two norbornene-type polymer backbones in PBS buffer with added thermolysin. Amino acid sequence is GPLGLAGGWGERDGS, a substrate for thermolysin.

FIG. 21. Chemical structure and synthesis of Protein-like Polymers (PLP) with different ABT898 peptide monomers. Different linkers were incorporated; Amide (A) and Amide RR (AR): Non-cleavable amide linker, Ester (E) and Ester RR (ER): cleavable ester linker. For each of structures Amide RR (AR) and Ester RR (ER) an additional two arginines (blue) were incorporated in the original sequence.

FIGS. 22A-22D. Characterization of norbornene functionalized ABT898 derivatives. ESI-MS and HPLC trace indicate the mass and purity of (FIG. 22A) ABT898, (FIG. 22B) ABT898 ester, (FIG. 22C) ABT898 RR, and (FIG. 22D) ABT898 Ester RR.

FIG. 23. Characterization of ABT898 PLPs: A PLP, E PLP, AR PLP, ER PLP. NMR is employed to determine polymerization kinetics. Mixture containing peptide and Grubbs second generation catalyst was monitored by NMR and checked for completion at 1-3 hours. Percent conversions were calculated from the integral values (polymer olefin)/(monomer olefin+polymer olefin) after subtracting the baseline integrals. SLS-MALS is used to determine Mn, DPI, and PDI for A PLP and E PLP.

FIGS. 24A-24D. Ex vivo choroidal sprouting assay. FIG. 24A. Schematic illustration of the assay. FIG. 24B. Time dependent assay of the growth area measured after the treatment of PLPs and peptides. FIG. 24C. Growth area upon the treatment of PLP, scramble version of PLP (AR PLP(S)), peptide, and aflibercept at day 4. FIG. 24D. Raw data: Light microscopy images of choroidal tissue upon treatment with buffer only, with parent peptide, scrambled PLP sequence, aflibercept, and the highly active ABT898 PLP with the amide linkage and two arginine residues included (AR PLP). Scale bar: 1 mm.

FIG. 25. ABT898 RR PLP (AR PLP) dose response curve displaying the extent of anti-angiogenic activity at various PLP concentrations.

FIGS. 26A-26B. Enzyme degradation kinetics and BLI assay. FIG. 26A. Percent enzymatic cleavage of peptide and PLP (AR PLP) upon the treatment of thermolysin (peptide:enzyme=2000:1) was monitored by HPLC over 450 min. FIG. 26B. Bio-layer interferometry data measuring the binding constant between AR PLP and CD36 receptor.

FIG. 27. Intraocular injections and pharmokinetic profile of rhodamine labeled AR PLP (AR PLP). Polymer is cleared from the vitreous fluid within 4 hours after injection.

FIG. 28. MALDI characterization of AR PLP at different concentrations performed to determine the limit of detection of polymer in the eye.

FIGS. 29A-29B. Representative chemical structures of norbornene modified Anti-VEGF derivative (a) and norbornene modified Anti-VEGF derivative (b), along with their corresponding HPLC traces and polymerization kinetics performed using NMR, are shown in FIG. 29A and FIG. 29B, respectively.

FIGS. 30A-30C. Ex vivo choroidal sprouting assay to evaluate the anti-angiogenic properties of anti-VEGF PLPs. FIG. 30A. Growth area upon the treatment of anti-VEGF amide PLP (57 μM and 115 μM), anti-VEGF ester PLP (57 μM and 115 μM), and aflibercept. FIG. 30B. Anti-VEGF Amide PLP and anti-VEGF ester PLP. 30C. Microscopic images of growth area upon treatment with anti-VEGF amide PLP, anti-VEGF ester PLP, and aflibercept.

FIGS. 31A-31D. Protein-Like Polymers (PLPs) as immunotherapeutic agents. FIG. 31A. Identification of tumor-specific mutations through next-generation sequencing allows for determination of promising peptide sequences that can be incorporated into PLPs as cancer vaccines. FIG. 31B. Synthesis of antigen containing PLPs using ROMP yields polymers with low dispersity and predetermined degrees of polymerization (TA: Terminating agent which can be either an adjuvant molecule or a fluorescent dye). FIG. 31C. In silico simulations show globular shapes resulting from peptide antigens side chains (colored ribbons) wrapping around the polymer backbone (gray) contributing to proteolytic resistance. FIG. 31D. Personalized PLPs-based vaccines can be administered alone or in combination with adjuvants to boost antitumor immune response.

FIGS. 32A-32B. Optimization of PLPs for in vitro cellular uptake and immune activation. FIG. 32A. Chemical structure of KVPRNQDWL (SEQ ID NO:10)-based PLPs conjugated to gp100 melanoma antigen via either an amide (A), ester (E) or disulfide (S) linker. FIG. 32B. Cellular uptake of fluorescently labeled PLPs by CD11c⁺ dendritic cells (DC) after 30 min incubation with various polymer concentrations (10, 50 and 100 μg/mL gp100). Representative experiment shown. Confocal fluorescence micrographs showing the uptake of PLP by splenic DC after 2 h incubation with PLP (S, 50 μg/mL gp100) (blue=DAPI, red=Rhodamine-labeled PLP). DC measuring CD86 expression was measured for indication of DC activation after 2 h incubation with A-, E-, and S-PLP (right, 50 μg/mL gp100). Representative flow panels are shown. Experiments performed in duplicate. All dosages normalized to amount of antigen. ***: p<0.005.

FIGS. 33A-33C. In vitro immune activation (T cells proliferation and cytokines expression) of pmel T cells after 3 days co-incubation with DC previously treated for 4 hours with either free gp100 (peptide) or PLPs (A, E and S, 10 μg/mL gp100). Proliferation and surface activation marker expression of CD8⁺ Pmel T cells was determined by flow cytometry. Homopolymers (m=15) bearing the three different linkers (A, E and S) were used for comparison in immune activation experiments. Dosages were normalized to amount of antigen. *: p<0.05, **: p<0.01, ***: p<0.005 as shown in FIG. 33A. Representative histograms for T cell proliferation (eFluor 450 dilution) at a ratio of 1:8 obtained from flow cytometry are shown in FIG. 33B. Comparison of surface activation marker CD69 on CD8+ Pmel T cells coincubated with different ratios of DCs previously treated with free peptide or gp100-PLPs is shown in FIG. 33C.

FIG. 34. T cell proliferation and cytokine expression in OT-1 splenocytes (recognizing specifically OVA-I) as determined by flow cytometry analysis upon incubation for 4 days with either free OVA-1 peptide or with OVA-1-conjugated PLP (S, 15 mer, 2 μg/mL OVA-1). PLPs of gp100 (copolymerized with PEG) used as negative controls here. Dosage normalized to the amount of antigen. *: p<0.05.

FIGS. 35A-35C. Stability of PLP homopolymers and PLP/PEG copolymers. FIGS. 35A and 35C. Proteolytic resistance of homopolymer (S, n=0 in FIG. 32A) and copolymer (S, n=5 in FIG. 32A) having different degrees of polymerization (n=15, 30 and 60). Trypsin was used as a model enzyme (1:1000 enzyme to substrate ratio) and experiments were performed in triplicate. FIG. 35B. Shows and exemplary KVPRNQDWL (SEQ ID NO:10)-based PLP/PEG copolymer.

FIGS. 36A-36D. In vivo activity of gp100-PLP (S—S linked) in combination with the STING agonist DMXAA. FIG. 36A. Structure of the copolymers used in this study (m=15 and m=30, n=5). FIG. 36B. Growth curves of B16F10 melanoma tumors treated with either 15 mer or 30 mer copolymers in combination with DMXAA. Treatments consisted of four subcutaneous injections containing both DMXAA (500 ug/injection) and either free gp100 peptide or PLPs (150 ug gp100/injection). 5 mice/group, ***: p<0.005. FIG. 36C. In vivo immune activation determined by flow cytometry of cells derived from tumor draining lymph nodes (TDLN) of mice treated with either PBS or PLP (30 mer+DMXAA). Representative flow panels are shown in FIG. 36D. PLP dosages are with respect to the amount of antigen. (*: p<0.05). That is, they are all normalized to be at equal amounts of peptides.

FIG. 37. Tumor growth inhibition and survival enhancement curves are shown for treatment with gp100-PLP in combination with 2′3′cGAMP in a B16F10 melanoma tumor model. B16F10 mice (n=6) were treated starting on day 7 post-inoculation with 30 mer gp100-PLP, (150 ug injection) in combination with the STING agonist 2′3′ cGAMP (injections 1,2) followed by two additional injections consisting of PLP only. While PLP was administrated subcutaneously, 2′3′cGAMP was injected intratumorally. **p<0.01 Monotherapy vs control mice; mice receiving combination therapy vs mice receiving monotherapy, or control mice.

FIG. 38. PLP treatment leads to expansion of T Cells with a central memory phenotype. Comparison of CD3 T cells coincubated with different ratios of DCs previously treated with either free gp100 (Peptide) or gp100-PLPs with antigen linked onto the polymer backbone using different linkage chemistries (A, E, S). CD44 and CD62L expression levels were compared. Representative flow panel shown. Dosages were normalized to amount of antigen 10 ug/ml gp100. ***: p<0.001.

FIG. 39. Comparison of DC activation using PLPs of different lengths and composition. Isolated DCs were treated overnight with different PLP formulations or LPS as positive control. S-15-Co: disulfide linked PLP, 15mer, copolymerized with 5 monomer units of PEG; S-30-Co: disulfide linked PLP, 30mer, copolymerized with 5 monomer units of PEG; S-60-Co: disulfide linked PLP, 60mer, copolymerized with 5 monomer units of PEG. Dosages were normalized to amount of antigen 50 ug/ml gp100. **: p<0.01.

FIG. 40. In vivo data showing blood pressure management in C57BL/6 mice injected with either terlipressin peptide or terlipressin PLP by measuring the systolic blood pressure over time by employing a tail-cuff system (CODA™).

### STATEMENTS REGARDING CHEMICAL COMPOUNDS AND NOMENCLATURE

The following abbreviations are used herein: MBHA refers to 4-methylbenzylhydrylamine; DMF refers to dimethylformaide; Acm refers to acetamidomethyl; TFA refers to trifluoroacetic acid; TIPS refers to triisopropyl silyl; RP-HPLC refers to reverse-phase high performance liquid chromatography; ESI-MS refers to electrospray ionization mass spectrometry; SEC-MALS refers to size-exclusion chromatography coupled with multiangle light scattering; and DP refers to degree of polymerization.

In an embodiment, a composition or compound of the invention is isolated or purified. In an embodiment, an isolated or purified compound is at least partially isolated or purified as would be understood in the art. In an embodiment, the composition or compound of the invention has a chemical purity of at least 95%, optionally for some applications at least 99%, optionally for some applications at least 99.9%, optionally for some applications at least 99.99%, and optionally for some applications at least 99.999% pure. The invention includes isolated and purified compositions of any of the brush block polymers described herein including terlipressin brush and block copolymers and brush and brush block copolymers having one or more side chains comprising terlipressin analogues, derivative, variants or fragments.

As used herein, the term “polymer” refers to a molecule composed of repeating structural units connected by covalent chemical bonds often characterized by a substantial number of repeating units (e.g., equal to or greater than 3 repeating units, optionally, in some embodiments equal to or greater than 5 repeating units, in some embodiments greater or equal to 10 repeating units) and a high molecular weight (e.g. greater than or equal to 20 Da, in some embodiments greater than or equal to 75 Da or greater than or equal to 100 Da). Polymers are commonly the polymerization product of one or more monomer precursors. The term polymer includes homopolymers, or polymers consisting essentially of a single repeating monomer subunit. The term polymer also includes copolymers which are formed when two or more different types of monomers are linked in the same polymer. Copolymers may comprise two or more monomer subunits, and include random, block, brush, brush block, alternating, segmented, grafted, tapered and other architectures. Useful polymers include organic polymers that may be in amorphous, semi-amorphous, crystalline or semi-crystalline states. Cross linked polymers having linked monomer chains are useful for some applications, for example linked by one or more disulfide linkages. The invention provides polymers comprising therapeutic agents, such as brush polymers having at least a portion of the repeating units comprising polymer side chains such as polypeptide side chains.

An “oligomer” refers to a molecule composed of repeating structural units connected by covalent chemical bonds often characterized by a number of repeating units less than that of a polymer (e.g., equal to or less than 3 repeating units) and a lower molecular weights (e.g. less than or equal to 1,000 Da) than polymers. Oligomers may be the polymerization product of one or more monomer precursors.

A “polypeptide” or “oligopeptide” herein are used interchangeably and refer to a polymer of repeating structural units connected by a peptide bond. Typically, the repeating structural units of the polypeptide are amino acids including naturally occurring amino acids, non-naturally occurring amino acids, analogues of amino acids or any combination of these. The number of repeating structural units of a polypeptide, as understood in the art, are typically less than a “protein”, and thus the polypeptide often has a lower molecular weight than a protein.

“Block copolymers” are a type of copolymer comprising blocks or spatially segregated domains, wherein different domains comprise different polymerized monomers, for example, including at least two chemically distinguishable blocks. Block copolymers may further comprise one or more other structural domains, such as hydrophobic groups, hydrophilic groups, etc. In a block copolymer, adjacent blocks are constitutionally different, i.e. adjacent blocks comprise constitutional units derived from different species of monomer or from the same species of monomer but with a different composition or sequence distribution of constitutional units. Different blocks (or domains) of a block copolymer may reside on different ends or the interior of a polymer (e.g. [A][B]), or may be provided in a selected sequence ([A][B][A][B]). “Diblock copolymer” refers to block copolymer having two different polymer blocks. “Triblock copolymer” refers to a block copolymer having three different polymer blocks, including compositions in which two non-adjacent blocks are the same or similar. “Pentablock” copolymer refers to a copolymer having five different polymer including compositions in which two or more non-adjacent blocks are the same or similar.

“Polymer backbone group” refers to groups that are covalently linked to make up a backbone of a polymer, such as a block copolymer. Polymer backbone groups may be linked to side chain groups, such as polymer side chain groups. Some polymer backbone groups useful in the present compositions are derived from polymerization of a monomer selected from the group consisting of a substituted or unsubstituted norbornene, olefin, cyclic olefin, norbornene anhydride, cyclooctene, cyclopentadiene, styrene and acrylate. Some polymer backbone groups useful in the present compositions are obtained from a ring opening metathesis polymerization (ROMP) reaction. Polymer backbones may terminate in a range of backbone terminating groups including hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —SOR³⁴, —OSR³⁵, —SO₂R³⁶, —OR³⁷, —SR³⁸, —NR³⁹R⁴⁰, NR⁴¹COR⁴², C₁-C₁₀ alkyl halide, phosphonate, phosphonic acid, silane, siloxane, acrylate, or catechol; wherein each of R³⁰-R⁴² is independently hydrogen, C₁-C₁₀ alkyl or C₅-C₁₀ aryl.

“Polymer side chain group” refers to a group covalently linked (directly or indirectly) to a polymer backbone group that comprises a polymer side chain, optionally imparting steric properties to the polymer. In an embodiment, for example, a polymer side chain group is characterized by a plurality of repeating units having the same, or similar, chemical composition. A polymer side chain group may be directly or indirectly linked to the polymer back bone groups. In some embodiments, polymer side chain groups provide steric bulk and/or interactions that result in an extended polymer backbone and/or a rigid polymer backbone. Some polymer side chain groups useful in the present compositions include unsubstituted or substituted polypeptide groups. Some polymer side chain groups useful in the present compositions comprise repeating units obtained via anionic polymerization, cationic polymerization, free radical polymerization, group transfer polymerization, or ring-opening polymerization. A polymer side chain may terminate in a wide range of polymer side chain terminating groups including hydrogen, C₁-C₁₀ alkyl, C₃-C₁₀ cycloalkyl, C₅-C₁₀ aryl, C₅-C₁₀ heteroaryl, acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —SOR³⁴, —OSR³⁵, —SO₂R³⁶, —OR³⁷, —SR³⁸, —NR³⁹R⁴⁰, —NR⁴¹COR⁴², C₁-C₁₀ alkyl halide, phosphonate, phosphonic acid, silane, siloxane acrylate, or catechol; wherein each of R³⁰-R⁴² is independently hydrogen or C₁-C₅ alkyl.

As used herein, the term “degree of polymerization” refers to the average number of monomer units per polymer chain. For example, for certain polymers described herein, comprising Z¹, Z², and/or S monomer units, the degree of polymerization would be represented by the sum total of Z¹, Z², and S monomer units. Since the degree of polymerization can vary from polymer to polymer, the degree of polymerization is generally represented by an average.

As used herein, the term “brush polymer” refers to a polymer comprising repeating units each independently comprising a polymer backbone group covalently linked to at least one polymer side chain group. A brush polymer may be characterized by brush density which refers to the percentage of the repeating units comprising polymer side chain groups. Brush polymers of certain aspects are characterized by a brush density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Brush polymers of certain aspects are characterized by a brush density selected from the range 50% to 100%, optionally some embodiments a density selected from the range of 75% to 100%, or optionally for some embodiments a density selected from the range of 90% to 100%.

As used herein, the term “peptide density” refers to the percentage of monomer units in the polymer chain which have a peptide covalently linked thereto. The percentage is based on the overall sum of monomer units in the polymer chain. For example, for certain polymers described herein, each P¹ is the polymer side chain comprising the peptide, each P² is a polymer side chain having a composition different from that of P¹, and each S is independently a repeating unit having a composition different from P¹ and P². Thus, the peptide density, or percentage of monomer units comprising the peptide (i.e., P¹ for this particular example) would be represented by the formula:

$$\frac{{P}^{1}}{{P}^{1} + {P}^{2} + S} \times 100 ,$$

where each variable refers to the number of monomer units of that type in the polymer chain.

In an aspect, the polymer side chain groups can have any suitable spacing on the polymer backbone. Typically, the space between adjacent polymer side chain groups is from 3 angstroms to 30 angstroms, and optionally 5 to 20 angstroms and optionally 5 to 10 angstroms. By way of illustration, in certain embodiments having a brush density of 100%, the polymer side chain groups typically are spaced 6±5 angstroms apart on the polymer backbone. In some embodiments the brush polymer has a high a brush density (e.g. greater than 70%), wherein the polymer side chain groups are spaced 5 to 20 angstroms apart on the polymer backbone

The term “sequence homology” or “sequence identity” means the proportion of amino acid matches between two amino acid sequences. When sequence homology is expressed as a percentage, e.g., 50%, the percentage denotes the fraction of matches over the length of sequence that is compared to some other sequence. Gaps (in either of the two sequences) are permitted to maximize matching; for example, wherein gap lengths of 5 amino acids or less, optionally 3 amino acids or less, are usually used.

The term “fragment” refers to a portion, but not all of, a composition or material, such as a polypeptide composition or material. In an embodiment, a fragment of a polypeptide refers to 50% or more of the sequence of amino acids, optionally 70% or more of the sequence of amino acids and optionally 90% or more of the sequence of amino acids.

“Polymer blend” refers to a mixture comprising at least one polymer, such as a brush polymer, e.g., brush block copolymer, and at least one additional component, and optionally more than one additional component. In some embodiments, for example, a polymer blend of the invention comprises a first brush copolymer and one or more addition brush polymers having a composition different than the first brush copolymer. In some embodiments, for example, a polymer blend of the invention further comprises one or more additional brush block copolymers, homopolymers, copolymers, block copolymers, brush block copolymers, oligomers, solvent, small molecules (e.g., molecular weight less than 500 Da, optionally less than 100 Da), or any combination of these. Polymer blends useful for some applications comprise a first brush polymer, and one or more additional components comprising polymers, block copolymers, brush polymers, linear block copolymers, random copolymers, homopolymers, or any combinations of these. Polymer blends of the invention include mixture of two, three, four, five and more polymer components.

As used herein, the term “group” may refer to a functional group of a chemical compound. Groups of the present compounds refer to an atom or a collection of atoms that are a part of the compound. Groups of the present invention may be attached to other atoms of the compound via one or more covalent bonds. Groups may also be characterized with respect to their valence state. The present invention includes groups characterized as monovalent, divalent, trivalent, etc. valence states.

As used herein, the term “substituted” refers to a compound wherein a hydrogen is replaced by another functional group.

Unless otherwise specified, the term “average molecular weight,” refers to number average molecular weight. Number average molecular weight is the defined as the total weight of a sample volume divided by the number of molecules within the sample. As is customary and well known in the art, peak average molecular weight and weight average molecular weight may also be used to characterize the molecular weight of the distribution of polymers within a sample.

As is customary and well known in the art, hydrogen atoms in formulas (FX1a)-(FX6b) are not always explicitly shown, for example, hydrogen atoms bonded to the carbon atoms of aromatic, heteroaromatic, and alicyclic rings are not always explicitly shown in formulas ((FX1a)-(FX6b). The structures provided herein, for example in the context of the description of formulas (FX1a)-(FX6b) and schematics and structures in the drawings, are intended to convey to one of reasonable skill in the art the chemical composition of compounds of the methods and compositions of the invention, and as will be understood by one of skill in the art, the structures provided do not indicate the specific positions and/or orientations of atoms and the corresponding bond angles between atoms of these compounds.

As used herein, the terms “alkylene” and “alkylene group” are used synonymously and refer to a divalent group derived from an alkyl group as defined herein. The invention includes compounds having one or more alkylene groups. Alkylene groups in some compounds function as linking and/or spacer groups. Compounds of the invention may have substituted and/or unsubstituted C₁-C₂₀ alkylene, alkylene and C₁-C₅ alkylene groups, for example, as one or more linking groups (e.g. L¹-L²).

As used herein, the terms “cycloalkylene” and “cycloalkylene group” are used synonymously and refer to a divalent group derived from a cycloalkyl group as defined herein. The invention includes compounds having one or more cycloalkylene groups. Cycloalkyl groups in some compounds function as linking and/or spacer groups. Compounds of the invention may have substituted and/or unsubstituted C₃-C₂₀ cycloalkylene, C₃-C₁₀ cycloalkylene and C₃-C₅ cycloalkylene groups, for example, as one or more linking groups (e.g. L¹-L²).

As used herein, the terms “arylene” and “arylene group” are used synonymously and refer to a divalent group derived from an aryl group as defined herein. The invention includes compounds having one or more arylene groups. In some embodiments, an arylene is a divalent group derived from an aryl group by removal of hydrogen atoms from two intra-ring carbon atoms of an aromatic ring of the aryl group. Arylene groups in some compounds function as linking and/or spacer groups. Arylene groups in some compounds function as chromophore, fluorophore, aromatic antenna, dye and/or imaging groups. Compounds of the invention include substituted and/or unsubstituted C₃-C₃₀ arylene, C₃-C₂₀ arylene, C₃-C₁₀ arylene and C₁-C₅ arylene groups, for example, as one or more linking groups (e.g. L¹-L²).

As used herein, the terms “heteroarylene” and “heteroarylene group” are used synonymously and refer to a divalent group derived from a heteroaryl group as defined herein. The invention includes compounds having one or more heteroarylene groups. In some embodiments, a heteroarylene is a divalent group derived from a heteroaryl group by removal of hydrogen atoms from two intra-ring carbon atoms or intra-ring nitrogen atoms of a heteroaromatic or aromatic ring of the heteroaryl group. Heteroarylene groups in some compounds function as linking and/or spacer groups. Heteroarylene groups in some compounds function as chromophore, aromatic antenna, fluorophore, dye and/or imaging groups. Compounds of the invention include substituted and/or unsubstituted C₃-C₃₀ heteroarylene, C₃-C₂₀ heteroarylene, heteroarylene and C₃-C₅ heteroarylene groups, for example, as one or more linking groups (e.g. L¹-L²).

As used herein, the terms “alkenylene” and “alkenylene group” are used synonymously and refer to a divalent group derived from an alkenyl group as defined herein. The invention includes compounds having one or more alkenylene groups. Alkenylene groups in some compounds function as linking and/or spacer groups. Compounds of the invention include substituted and/or unsubstituted C₂-C₂₀ alkenylene, C₂-C₁₀ alkenylene and C₂-C₅ alkenylene groups, for example, as one or more linking groups (e.g. L¹-L²).

As used herein, the terms “cycloalkenylene” and “cycloalkenylene group” are used synonymously and refer to a divalent group derived from a cycloalkenyl group as defined herein. The invention includes compounds having one or more cycloalkenylene groups. Cycloalkenylene groups in some compounds function as linking and/or spacer groups. Compounds of the invention include substituted and/or unsubstituted C₃-C₂₀ cycloalkenylene, C₃-C₁₀ cycloalkenylene and C₃-C₅ cycloalkenylene groups, for example, as one or more linking groups (e.g. L¹-L²).

As used herein, the terms “alkynylene” and “alkynylene group” are used synonymously and refer to a divalent group derived from an alkynyl group as defined herein. The invention includes compounds having one or more alkynylene groups. Alkynylene groups in some compounds function as linking and/or spacer groups. Compounds of the invention include substituted and/or unsubstituted C₂-C₂₀ alkynylene, C₂-C₁₀ alkynylene and C₂-C₅ alkynylene groups, for example, as one or more linking groups (e.g. L¹-L²).

As used herein, the term “halo” refers to a halogen group such as a fluoro (—F), chloro (—Cl), bromo (—Br), iodo (—I) or astato (—At).

The term “heterocyclic” refers to ring structures containing at least one other kind of atom, in addition to carbon, in the ring. Examples of such heteroatoms include nitrogen, oxygen and sulfur. Heterocyclic rings include heterocyclic alicyclic rings and heterocyclic aromatic rings. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and functional groups, for example, provided as substituents.

The term “carbocyclic” refers to ring structures containing only carbon atoms in the ring. Carbon atoms of carbocyclic rings can be bonded to a wide range of other atoms and functional groups, for example, provided as substituents.

The term “alicyclic ring” refers to a ring, or plurality of fused rings, that is not an aromatic ring. Alicyclic rings include both carbocyclic and heterocyclic rings.

The term “aromatic ring” refers to a ring, or a plurality of fused rings, that includes at least one aromatic ring group. The term aromatic ring includes aromatic rings comprising carbon, hydrogen and heteroatoms. Aromatic ring includes carbocyclic and heterocyclic aromatic rings. Aromatic rings are components of aryl groups.

The term “fused ring” or “fused ring structure” refers to a plurality of alicyclic and/or aromatic rings provided in a fused ring configuration, such as fused rings that share at least two intra ring carbon atoms and/or heteroatoms.

As used herein, the term “alkoxyalkyl” refers to a substituent of the formula alkyl-O-alkyl.

As used herein, the term “polyhydroxyalkyl” refers to a substituent having from 2 to 12 carbon atoms and from 2 to 5 hydroxyl groups, such as the 2,3-dihydroxypropyl, 2,3,4-trihydroxybutyl or 2,3,4,5-tetrahydroxypentyl residue.

As used herein, the term “polyalkoxyalkyl” refers to a substituent of the formula alkyl-(alkoxy)ₙ-alkoxy wherein n is an integer from 1 to 10, preferably 1 to 4, and more preferably for some embodiments 1 to 3.

Amino acids include glycine, alanine, valine, leucine, isoleucine, methionine, proline, phenylalanine, tryptophan, asparagine, glutamine, glycine, serine, threonine, serine, rhreonine, asparagine, glutamine, tyrosine, cysteine, lysine, arginine, histidine, aspartic acid and glutamic acid. As used herein, reference to “a side chain residue of a natural α-amino acid” specifically includes the side chains of the above-referenced amino acids. Peptides are comprised of two or more amino acids connected via peptide bonds.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. The term cycloalkyl specifically refers to an alky group having a ring structure such as ring structure comprising 3-30 carbon atoms, optionally 3-20 carbon atoms and optionally 2-10 carbon atoms, including an alkyl group having one or more rings. Cycloalkyl groups include those having a 3-, 4-, 5-, 6-, 7-, 8-, 9- or 10-member carbon ring(s) and particularly those having a 3-, 4-, 5-, 6-, or 7-member ring(s). The carbon rings in cycloalkyl groups can also carry alkyl groups. Cycloalkyl groups can include bicyclic and tricycloalkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups. As used herein MeO— refers to CH₃O—. Compositions of some embodiments of the invention comprise alkyl groups as terminating groups, such as polymer backbone terminating groups and/or polymer side chain terminating groups.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6- or 7-member ring(s). The carbon rings in cycloalkenyl groups can also carry alkyl groups. Cycloalkenyl groups can include bicyclic and tricyclic alkenyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogen atoms replaced with one or more fluorine atoms. Compositions of some embodiments of the invention comprise alkenyl groups as terminating groups, such as polymer backbone terminating groups and/or polymer side chain terminating groups.

Aryl groups include groups having one or more 5-, 6- or 7-member aromatic rings, including heterocyclic aromatic rings. The term heteroaryl specifically refers to aryl groups having at least one 5-, 6- or 7-member heterocyclic aromatic rings. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those which are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein are provided in a covalently bonded configuration in the compounds of the invention at any suitable point of attachment. In embodiments, aryl groups contain between 5 and 30 carbon atoms. In embodiments, aryl groups contain one aromatic or heteroaromatic six-membered ring and one or more additional five- or six-membered aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents. Compositions of some embodiments of the invention comprise aryl groups as terminating groups, such as polymer backbone terminating groups and/or polymer side chain terminating groups.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms. Compositions of some embodiments of the invention comprise arylalkyl groups as terminating groups, such as polymer backbone terminating groups and/or polymer side chain terminating groups.

As to any of the groups described herein which contain one or more substituents, it is understood that such groups do not contain any substitution or substitution patterns which are sterically impractical and/or synthetically non-feasible. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for any alkyl, alkenyl and aryl group includes substitution with one or more of the following substituents, among others: halogen, including fluorine, chlorine, bromine or iodine; pseudohalides, including —CN;

—COOR where R is a hydrogen or an alkyl group or an aryl group and more specifically where R is a methyl, ethyl, propyl, butyl, or phenyl group all of which groups are optionally substituted;

—COR where R is a hydrogen or an alkyl group or an aryl group and more specifically where R is a methyl, ethyl, propyl, butyl, or phenyl group all of which groups are optionally substituted;

—CON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—OCON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—N(R)₂ where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring which can contain one or more double bonds and can contain one or more additional carbon atoms;

—SR, where R is hydrogen or an alkyl group or an aryl group and more specifically where R is hydrogen, methyl, ethyl, propyl, butyl, or a phenyl group, which are optionally substituted;

—SO₂R, or —SOR where R is an alkyl group or an aryl group and more specifically where R is a methyl, ethyl, propyl, butyl, or phenyl group, all of which are optionally substituted;

—OCOOR where R is an alkyl group or an aryl group;

—SO₂N(R)₂ where each R, independently of each other R, is a hydrogen, or an alkyl group, or an aryl group all of which are optionally substituted and wherein R and R can form a ring which can contain one or more double bonds and can contain one or more additional carbon atoms;

—OR where R is H, an alkyl group, an aryl group, or an acyl group all of which are optionally substituted. In a particular example R can be an acyl yielding —OCOR″ where R″ is a hydrogen or an alkyl group or an aryl group and more specifically where R″ is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

As to any of the above groups which contain one or more substituents, it is understood that such groups do not contain any substitution or substitution patterns which are sterically impractical and/or synthetically non-feasible.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or D- or L-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms. Isomers include structural isomers and stereoisomers such as enantiomers.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to a subject, such as a patient in need of treatment; slowing in the rate of degeneration or decline; making the final point of degeneration less debilitating; improving a subject's physical or mental well-being. The treatment or amelioration of symptoms can be based on objective or subjective parameters; including the results of a physical examination, neuropsychiatric exams, and/or a psychiatric evaluation.

An “effective amount” is an amount sufficient to accomplish a stated purpose (e.g. achieve the effect for which it is administered, treat a disease, reduce enzyme activity, increase enzyme activity, reduce transcriptional activity, increase transcriptional activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist (inhibitor) required to decrease the activity of an enzyme or protein (e.g. transcription factor) relative to the absence of the antagonist. An “activity increasing amount,” as used herein, refers to an amount of agonist (activator) required to increase the activity of an enzyme or protein (e.g. transcription factor) relative to the absence of the agonist. A “function disrupting amount,” as used herein, refers to the amount of antagonist (inhibitor) required to disrupt the function of an enzyme or protein (e.g. transcription factor) relative to the absence of the antagonist. A “function increasing amount,” as used herein, refers to the amount of agonist (activator) required to increase the function of an enzyme or protein (e.g. transcription factor) relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As defined herein, the term “inhibition”, “inhibit”, “inhibiting” and the like in reference to a protein-inhibitor (e.g. antagonist) interaction means negatively affecting (e.g. decreasing) the activity or function of the protein relative to the activity or function of the protein in the absence of the inhibitor. In some embodiments inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g. agonist) interaction means positively affecting (e.g. increasing) the activity or function of the protein

The term “modulator” refers to a composition that increases or decreases the level of a target molecule or the function of a target molecule.

“Patient” or “subject in need thereof” refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a mammal. In some embodiments, a patient is a mouse. In some embodiments, a patient is an experimental animal. In some embodiments, a patient is a rat. In some embodiments, a patient is a test animal.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). In embodiments, administration includes direct administration to a tumor. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent or chemotherapeutic). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

As used herein, the term “conjugated” when referring to two moieties means the two moieties are bonded, wherein the bond or bonds connecting the two moieties may be covalent or non-covalent. In embodiments, the two moieties are covalently bonded to each other (e.g. directly or through a covalently bonded intermediary). In embodiments, the two moieties are non-covalently bonded (e.g. through ionic bond(s), van der waal's bond(s)/interactions, hydrogen bond(s), polar bond(s), or combinations or mixtures thereof).

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

In an aspect, the invention provides a polymer comprising a polymeric backbone which is directly or indirectly covalently linked to a polymer side chain. The inventive polymer can comprise, or be derived from, any suitable number of monomers. For example, in some embodiments, the polymer is a homopolymer (i.e., derived from one type of monomer). Alternatively, in some embodiments, the polymer can be a copolymer comprising (e.g., derived from) more than one type of monomer.

It will be understood that the inventive polymer, along with the linked polymer side chains, can have any suitable configuration. For example, in some embodiments wherein the polymer is a homopolymer, the polymer can be a brush polymer. In other embodiments wherein the polymer is a copolymer, the polymer can be a brush block copolymer.

In an embodiment, the polymer comprises a first polymer block comprising at least 2 first repeating units, and optionally at least 5 first repeating units; wherein each of the first repeating units of the first polymer block comprises a first polymer backbone group directly or indirectly covalently linked to a first polymer side chain group comprising a therapeutic peptide, such as a vasoconstrictor peptide.

In keeping with an aspect of the invention, the polymer side chain comprises a therapeutic peptide (i.e., oligopeptide or polypeptide). The polypeptide comprises a suitable number of amino acid units. In keeping with an aspect of the invention, the therapeutic peptide comprises at least two amino acid units. For example, the peptide comprises 2 or more amino acid units, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, or 30 or more amino acid units. Alternatively, or in addition, the peptide can comprise 100 or less amino acid units, for example, 90 or less, 80 or less, 70 or less, 60 or less, 59 or less, 58 or less, 57 or less, 56 or less, 55 or less, 54 or less, 53 or less, 52 or less, 51 or less, 50 or less, 49 or less, 48 or less, 47 or less, 46 or less, 45 or less, 44 or less, 43 or less, 42 or less, 41 or less, 40 or less, 39 or less, 38 or less, 37 or less, 36 or less, 35 or less, 34 or less 33 or less, 32 or less, or 31 or less amino acid units. Thus, the peptide can comprise a number of amino acid units bounded by any two of the aforementioned endpoints. For example, the peptide can comprise 2 to 100 amino acid units, for example, 2 to 90, 2 to 80, 2 to 70, 2 to 60, 5 to 100, 5 to 90, 5 to 80, 5 to 70, 5 to 60, 3 to 60, 3 to 59, 4 to 58, 5 to 57, 6 to 56, 7 to 55, 8 to 54, 9 to 53, 10 to 52, 11 to 51, 12 to 50, 13 to 49, 14 to 48, 15 to 47, 16 to 46, 17 to 45, 18 to 44, 19 to 43, 20 to 42, 21 to 41, 22 to 42, 23 to 41, 24 to 40, 25 to 39, 26 to 38, 27 to 37, 28 to 36, 29 to 35, 30 to 34, or 31 to 33 amino acid units. In certain embodiments, the peptide comprises 5 to 100 amino acids. In preferred embodiments, the peptide comprises 8 to 60 amino acid.

The peptide can have any suitable structure (e.g. primary, secondary, tertiary, or quaternary structure). For example, in some embodiments, the therapeutic peptide is a branched or linear polypeptide. In some embodiments, the peptide is a cross-linked polypeptide

In some specific embodiments, the peptide is peptide or polypeptide exhibiting in vivo activity. In an embodiment the peptide comprises, consists essentially of, consists of, or is an analogue of a terlipressin peptide, an anti-VEGF peptide, an ABT-898 peptide, a neoantigen-Melanoma peptide, an antigen-M30 peptide, an antigen-gp100 Melanoma peptide or a derivative, a variant, an analogue, or a fragment of terlipressin peptide, an anti-VEGF peptide, an ABT-898 peptide, a neoantigen-melanoma peptide, an antigen-M30 peptide, or an antigen-gp100 Melanoma peptide.

Suitable derivatives include, for example, peptide conjugates including conjugates with one or more linker moieties such as cleavable, degradable or triggerable linkers.

In embodiments wherein the therapeutic peptide is a fragment of a vasopressin analogue, the fragment can be any suitable fragment. For example, in embodiments when the therapeutic peptide.

The fragment can be any suitable fragment, for example 2-8 amino acid units, for example, 8 or less, 7 or less, 6 or less, 5 or less, 4 or less, 3 or less, or 2 amino acid units.

In keeping with an aspect of the invention, the therapeutic peptide is linked to the polymer backbone. In some embodiments, the therapeutic peptide is linked to the polymer backbone via an enzymatically degradable linker (i.e., linking group or linking moiety). Examples of suitable cleavable, degradable or triggerable linkers include enzyme cleavable sequences such as one or more matrix metalloproteinase (MMP) cleavage sequence, cathepsin B cleavage sequence, ester bond, reductive sensitive bond—disulfide bond, pH sensitive bond—imine bond, among others

In some embodiments, the polymer is characterized by the formula (FX1a), (FX1b), (FX1c), (FX1d); (FX1e); (FX1f); or (FX1g):

Q¹-T-Q² (FX1a);

Q¹-T-[S]ₕ-Q² (FX1b);

Q¹-[S]ₕ-T-Q² (FX1c);

Q¹-[S]ᵢ-T-[S]ₕ-Q² (FX1d);

Q¹-[S]ᵢ-T-[S]ₕ-T-Q² (FX1e);

Q¹-T-[S]ᵢ-T-[S]ₕ-Q² (FX1f); or

Q¹-T-[S]ᵢ-T-[S]ₕ-T-Q² (FX1g);

wherein each T is independently the first polymer block comprising the first repeating units and each S is independently an additional polymer block; Q¹ is a first polymer block terminating group; Q² is a second polymer block terminating group; and wherein h is zero or an integer selected over the range of 1 to 1000 (e.g., 1 to 500, 1 to 250, 1 to 100, or 1 to 50) and i is zero or an integer selected over the range of 1 to 1000 (e.g., 1 to 500, 1 to 250, 1 to 100, or 1 to 50). In an embodiment, the polymer is characterized by any of formulas (FX1a)-(FX1g), wherein each -T- is independently -[Y¹]ₘ-; wherein each Y¹ is independently the first repeating unit of the first polymer block; and each m is independently an integer selected from the range 0 to 1000 (e.g., 0 or 1 to 500, 1 to 250, 1 to 100, or 1 to 50), provided that at least one m is an integer selected from the range 1 to 1000 (e.g., 1 to 500, 1 to 250, 1 to 100, or 1 to 50).

In certain embodiments, the polymer is characterized by the formula (FX2a), (FX2b), or (FX2c):

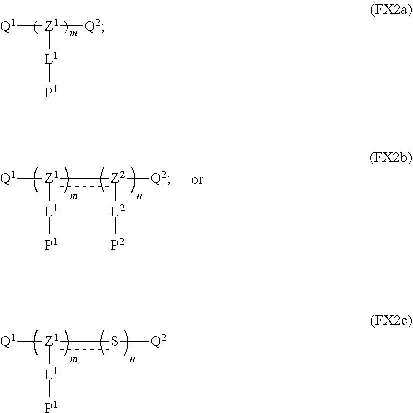

wherein each Z¹ is independently a first polymer backbone group and each Z² is independently a second polymer backbone group; wherein each S is independently a repeating unit having a composition different from the first repeating unit; the wherein Q¹ is a first backbone terminating group and Q² is a second backbone terminating group; wherein each Lis independently a first linking group, each L² is independently a second linking group; wherein each P¹ is the polymer side chain comprising the peptide; wherein each P² is a polymer side chain having a composition different from that of P¹; and wherein each m is independently an integer selected from the range of 2 to 1000 (e.g., 2 to 500, 2 to 250, 2 to 100, or 2 to 50); wherein each n is each independently an integer selected from the range of 0 to 1000 (e.g., 0 to 500, 0 to 250, 0 to 100, or 0 to 50); and wherein h are each independently an integer selected from the range of 0 to 1000 (e.g., 0 to 500, 0 to 250, 0 to 100, or 0 to 50).

For each of the polymers characterized by the formula (FX2a), (FX2b), and (FX2c), each of Z¹ and Z² can be any suitable monomer capable of undergoing ring opening metathesis or cross metathesis. For example, each of Z¹ and Z² can independently be a substituted or unsubstituted norbornene, oxanorbornene, olefin, cyclic olefin, cyclooctene, or cyclopentadiene. In some embodiments, each of the first polymer backbone group and/or the second polymer backbone group is a polymerized norbornene dicarboxyimide monomer. In preferred embodiments, each polymer backbone group of the polymer is a polymerized norbornene dicarboxyimide monomer.

Thus, for each of the polymers characterized by the formula (FX2a), (FX2b), and (FX2c), each Z¹ connected to L¹, and P¹ or a combination thereof can independently be characterized by the formula (FX3a) or (FX3b):

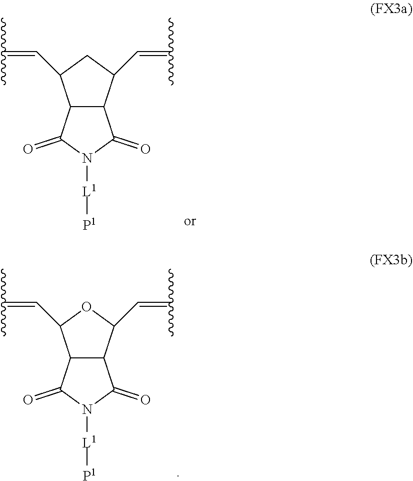

and when present, each Z² connected to L², and P² or a combination thereof can independently be characterized by the formula (FX4a) or (FX4b)

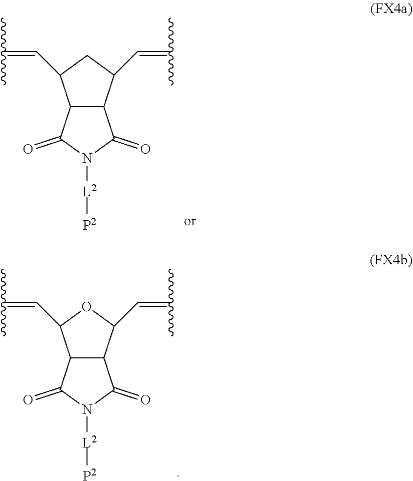

In certain embodiments of the polymers characterized by the formula (FX2a), (FX2b), and (FX2c), each Z1 connected to L1, and P1 or a combination thereof is independently characterized by the formula (FX3a):

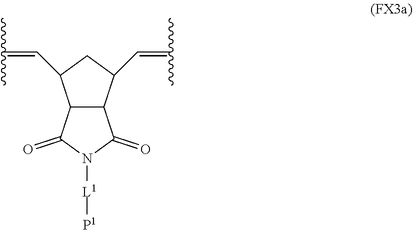

and/or each Z² connected to L², and P² or a combination thereof is independently characterized by the formula (FX4a):

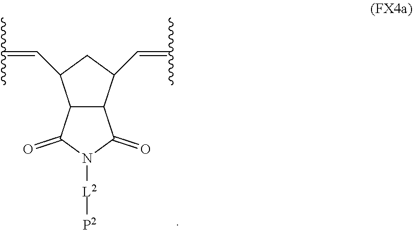

For each of the polymers characterized by the formula (FX1a), (FX1b), (FX1c), (FX1d), (FX1e), (FX1f), (FX1g), (FX2a), (FX2b), and (FX2c), each of Q¹ and Q² can independently be selected from a hydrogen, C₁-C₃₀ alkyl, C₃-C₃₀ cycloalkyl, C₅-C₃₀ aryl, C₅-C₃₀ heteroaryl, C₁-Cao acyl, C₁-C₃₀ hydroxyl, C₁-C₃₀ alkoxy, C₂-C₃₀ alkenyl, C₃₀ alkynyl, C₅-C₃₀ alkylaryl, —CO₂R³, —CONR⁴R⁵, —COR⁶, —SOR⁷, —OSR⁸, —SO₂R⁹, —OR¹⁰, —SR¹¹, —NR¹²R¹³, —NR¹⁴COR¹⁵, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R³-R¹⁵ is independently H, C₅-C₁₀ aryl or C₁-C₁₀ alkyl.

For each of the polymers characterized by the formula (FX2a), (FX2b), and (FX2c), each of L¹ and L² can be any suitable linking group. For example, each of L¹ and L² can independently be selected from a single bond, an oxygen, and groups having an alkyl group, an alkenylene group, an arylene group, an alkoxy group, an acyl group, a triazole group, a diazole group, a pyrazole group, and combinations thereof. In certain embodiments, each of L¹ and L² is independently selected from a single bond, —O—, C₁-C₁₀ alkyl, C₂-C₁₀ alkenylene, C₃-C₁₀ arylene, alkoxy, acyl and combinations thereof.

For each of the polymers characterized by the formula (FX2a), (FX2b), and (FX2c), each P² is a polymer side chain having a composition different from that of P¹. Thus, P² can be any suitable side chain capable of being incorporated into the polymer with P¹. In some embodiments, P² is a peptide or protein other than P¹. Thus, the polymer can comprise two different peptide or protein units. In some embodiments, P² is a nonionic polymer selected from a polyalkylene glycol, a polyetheramine, a polyethylene oxide/polypropylene oxide copolymer, a polysaccharide, and combinations thereof. In certain embodiments, the nonionic polymer is a polyalkylene glycol (e.g., polyethylene glycol (PEG) or polypropylene oxide (PPO)), a polyethylene oxide/polypropylene oxide copolymer, or a combination thereof. In preferred embodiments, the nonionic polymer is a polyethylene glycol (PEG). Thus, in some embodiments, each Z² connected to L², and P² or a combination thereof is independently characterized by the formula (FX7a) or (FX7b):

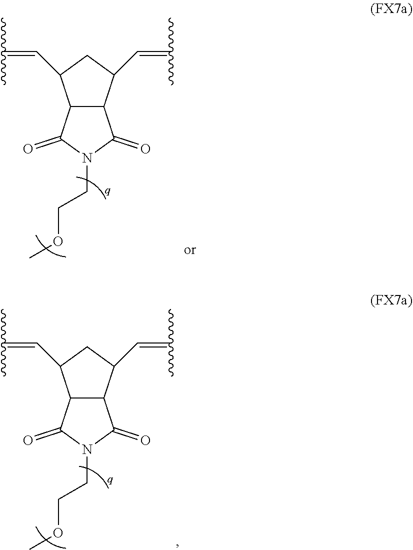

wherein q is an integer from 1 to 500 (e.g., 1 to 250, 1 to 100, 1 to 50, 1 to 25, 1 to 10, or 1 to 6).

For each of the polymers characterized by the formula (FX2a), (FX2b), and (FX2c), each S is independently a repeating unit having a composition different from the first repeating unit. Thus, S can be any monomer unit capable of being incorporated into the polymer with P¹. In some embodiments, S comprises a nonionic polymer selected from a polyalkylene glycol, a polyetheramine, a polyethylene oxide/polypropylene oxide copolymer, a polysaccharide, and combinations thereof. In certain embodiments, the nonionic polymer is a polyalkylene glycol (e.g., polyethylene glycol (PEG) or polypropylene oxide (PPO)), a polyethylene oxide/polypropylene oxide copolymer, or a combination thereof. In preferred embodiments, the nonionic polymer is a polyethylene glycol (PEG). Thus, in some embodiments, each S is independently characterized by the formula (FX7a) or (FX7b):

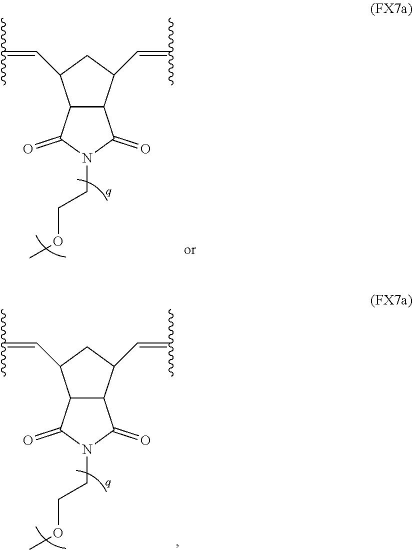

wherein q is an integer from 1 to 500 (e.g., 1 to 250 1 to 100, 1 to 50, 1 to 25, 1 to 10, or 1 to 6)

In certain embodiments, the polymer is characterized by the formula (FX2a), (FX2b), or (FX2c):

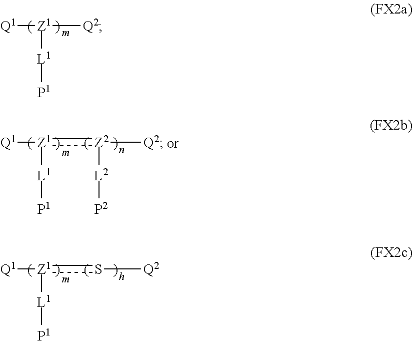

wherein each Z¹ is independently a first polymer backbone group and each Z² is independently a second polymer backbone group; each S is independently a repeating unit having a composition different from the first repeating unit; Q¹ is a first backbone terminating group and Q² is a second backbone terminating group; each Lis independently a first linking group, each L² is independently a second linking group; each P¹ is the polymer side chain comprising the peptide; wherein each P² is a polymer side chain having a composition different from that of P¹; each m is independently an integer selected from the range of 2 to 100; each n is independently an integer selected from the range of 0 to 100; and each h is independently an integer selected from the range of 0 to 100, provided that each of the first polymer backbone group and/or the second polymer backbone group is a polymerized norbornene dicarboxyimide monomer, and wherein the polymer fulfills at least one (i.e., (i), (ii), and/or (iii) of the following properties:
(i) the polymer has a degree of polymerization of 5 to 100,
(ii) the peptide comprises 5 to 100 amino acids, and
(iii) the polymer has a peptide density of greater than 50%, as defined by the following formula:

$$\frac{{P}^{1}}{{P}^{1} + {P}^{2} + S} \times 100.$$

In preferred embodiments, the polymer fulfills all of properties (i)-(iii) above.

After polymerization the inventive polymers may be characterized using any suitable technique(s). Typically, the inventive polymers are characterized by size-exclusion chromatography with multiangle light scattering (SEC-MALS) to ascertain degree of polymerization (DP) and molecular weight distribution (dispersity or Mw/Mn). Preferably, there is suitable agreement between the obtained DP and the theoretical DP based on the initial monomer-to-initiator ratio ([M]₀/[I]₀).

The inventive polymer can have any suitable degree of polymerization. If the degree of polymerization is too low, the polymer may not be resistant to enzymatic cleavage by proteases or may be cleared too rapidly from the body since the polymer's molecular weight would be lower than the clearance threshold through the kidney. Alternatively, if the degree of polymerization is too high, the peptide side chain groups displayed on the polymer may be too dense to engage their biological targets such as cell receptors, enzymes, etc. Typically, the polymer has a degree of polymerization of 2 to 1000 (e.g., 2 to 500, 2 to 250, 2 to 100, 2 to 50, 5 to 1000, 5 to 500, 5 to 250, 5 to 100, or 5 to 50). In certain embodiments, the polymer has a degree of polymerization of 5 to 100. In preferred embodiments, the polymer has a degree of polymerization of 5 to 50. For example, the polymer can have a degree of polymerization of 5 or about 5, a degree of polymerization of 15 or about 15 (e.g., 17), a degree of polymerization of 30 or about 30, or a degree of polymerization of 50 or about 50.

The inventive polymer can have any suitable weight average molecular weight. The polymers can have a weight average molecular weight of 2,000 kDa or less, for example, 1,800 kDa or less, 1,600 kDa or less, 1,400 kDa or less, 1,200 kDa or less, 1,000 kDa or less, 900 kDa, or less, 800 kDa, or less, 700 kDa or less, 600 kDa or less, 500 kDa or less, 250 kDa or less, 100 kDa or less, or 50 kDa or less. Alternatively, or in addition, the polymers can have a weight average molecular weight of 500 Da or more, for example, 1 kDa or more, 5 kDa or more, or 10 kDa or more. Thus, the polymers can have a weight average molecular weight bounded by any two of the aforementioned endpoints. For example, the polymers can have a weight average molecular weight of from 500 Da to 2,000 kDa, from 500 Da to 1,000 kDa, from 500 Da to 500 kDa, from 500 Da to 100 kDa, from 500 Da to 50 kDa, 1 kDa to 2,000 kDa, from 1 kDa to 1,000 kDa, from 1 kDa to 500 kDa, from 1 kDa to 100 kDa, from 1 kDa to 50 kDa, 5 kDa to 2,000 kDa, from 5 kDa to 1,000 kDa, from 5 kDa to 500 kDa, from 5 kDa to 100 kDa, from 5 kDa to 50 kDa, 10 kDa to 2,000 kDa, from 10 kDa to 1,000 kDa, from 10 kDa to 500 kDa, from 10 kDa to 100 kDa, or from 10 kDa to 50 kDa.

The polymer can have any suitable peptide density. The polymer may be characterized by peptide density which refers to the percentage of the repeating units comprising a polymer backbone group covalently linked to at least one peptide. Thus, for each of the polymers characterized by the formula (FX2a), (FX2b), and (FX2c), the polymer density can be defined by the following formula:

$$\frac{{P}^{1}}{{P}^{1} + {P}^{2} + S} \times 100.$$

Generally, the polymers described herein are characterized by a peptide density of greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Brush polymers of certain aspects are characterized by a peptide density selected from the range 50% to 100%, optionally some embodiments a peptide density selected from the range of 60% to 100%, optionally for some embodiments a peptide density selected from the range of 70% to 100%, optionally some embodiments a peptide density selected from the range of 80% to 100%, or optionally for some embodiments a peptide density selected from the range of 90% to 100%.

In another aspect, the invention provides a pharmaceutical composition comprising one or more polymers described herein. In some embodiments, the composition comprises one or more pharmaceutically acceptable excipients. For example, the polymers of the invention can be formulated for parenteral administration, such as intravenous (IV) administration or administration into a body cavity or lumen of an organ. Alternatively, the polymers can be injected intra-tumorally. Formulations for injection will commonly comprise a solution of the polymer dissolved in a pharmaceutically acceptable carrier. Among the acceptable vehicles and solvents that can be employed are water and an isotonic sodium chloride. In addition, sterile fixed oils can conventionally be employed as a solvent or suspending medium. For this purpose, any bland fixed oil can be employed including synthetic monoglycerides or diglycerides. In addition, fatty acids such as oleic acid can likewise be used in the preparation of injectables. These solutions are sterile and generally free of undesirable matter. These formulations can be sterilized by conventional, well known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the polymer in these formulations can vary widely, and will be selected primarily based on fluid volumes, viscosities, body weight, and the like, in accordance with the particular mode of administration selected and the patient's needs. In certain embodiments, the concentration of a polymer in a solution formulation for injection will range from 0.1% (w/w) to 10% (w/w) or about 0.1% (w/w) to about 10% (w/w).

In some embodiments, the composition further comprises an additional immune-stimulatory compound such as small molecule STING agonists, anti-PD-1/PD-L1 peptides, or a pattern recognition receptor (PRR) agonists. As used herein, the terms “Pattern recognition receptor” and “PRR” refer to any member of a class of conserved mammalian proteins which recognize pathogen-associated molecular patterns (“PAMPs”) or damage-associated molecular patterns (“DAMPs”), and act as key signaling elements in innate immunity. Pattern recognition receptors are divided into membrane-bound PRRs, cytoplasmic PRRs, and secreted PRRs. Examples of membrane-bound PRRs include Toll-like receptors (“TLRs”) and C-type lectin receptors (“CLRs”). Examples of cytoplasmic PRRs include NOD-like receptors (“NLRs”) and Rig-I-like receptors (“RLRs”). In some embodiments, the compositions can have more than one additional immune-stimulatory compound.

Additional immune-stimulatory compounds, such as cytosolic DNA and unique bacterial nucleic acids called cyclic dinucleotides, can be recognized by stimulator of interferon genes (“STING”), which can act a cytosolic DNA sensor. ADU-SIOO can be a STING agonist. Additional, non-limiting examples of STING agonists include: Cyclic [G(2′,5′)pA(2′,5′)p] (2′2′-cGAMP), cyclic [G(2′,5′)pA(3′,5′)p] (2′3′-cGAMP), cyclic [G(3′,5′)pA(3′,5′)p] (3′3′-cGAMP), Cyclic di-adenylate monophosphate (c-di-AMP), 2′,5′-3′,5′-c-diAMP (2′3′-c-di-AMP), Cyclic di-guanylate monophosphate (c-di-GMP), 2′,5′-3′,5′-c-diGMP (2′3′-c-di-GMP), Cyclic di-inosine monophosphate (c-di-IMP), Cyclic di-uridine monophosphate (c-di-UMP), KIN700, KIN1148, KIN600, KIN500, KINIOO, KIN101, KIN400, KIN2000, or SB-9200 can be recognized.

In another aspect, the invention provides a method for treating a disease described herein or a disease associated with a protein described herein. The method includes administering a therapeutically effective amount of a polymer or a composition described herein to a subject in need thereof. For example, the methods can include administering the polymer to provide a dose of from 10 ng/kg to 50 mg/kg to the subject. For example, the polymer dose can range from 5 mg/kg to 50 mg/kg, from 10 μg/kg to 5 mg/kg, or from 100 μg/kg to 1 mg/kg. The polymer dose can also lie outside of these ranges, depending on the particular polymer as well as the type of disease being treated. Frequency of administration can range from a single dose to multiple doses per week, or more frequently. In some embodiments, the polymer is administered from about once per month to about five times per week. In some embodiments, the polymer is administered once per week.

The invention may be further set forth and understood in view of the following non-limiting examples and embodiments which one having skill in the art will readily understand are intended to illustrate specific aspects of the invention.

### Example 1—Terlipressin Brush Polymers

This example describes exemplary brush polymers comprising a terlipressin peptide, or a derivative, analogue, variant, isomer or fragment of a terlipressin peptide and related methods for diverse therapeutic applications including vasoconstrictive therapy and/or the treatment or maintenance of a condition such as hepatorenal syndrome, low blood pressure, bleeding esophageal varices, septic shock or paracentesis-induced circulatory dysfunction.

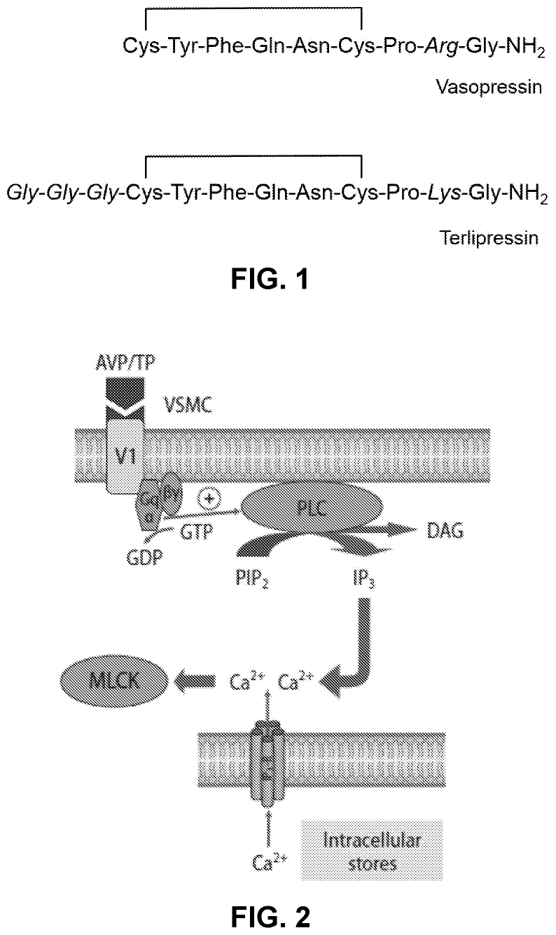

FIG. 1 depicts the primary structures of vasopressin and terlipressin. Terlipressin is an analogue of the naturally occurring peptide vasopressin that causes narrowing of blood vessels (vasoconstriction). Terlipressin is a registered drug in Europe, Australia and parts of Asia, prescribed for patients with bleeding esophageal varices (bleeding from dilated veins in the food pipe leading to the stomach).

If the vessels in the liver are blocked due to liver damage, blood cannot flow properly through the liver. As a result, high pressure in the portal system develops. This increased pressure in the portal vein may lead to the development of large, swollen veins (varices) within the esophagus, stomach, rectum, or umbilical area. Varices can rupture and bleed, resulting in potentially life-threatening complications. Bleeding of esophageal varices is one of the most dramatic complications in gastroenterology and has a 20-50% mortality rate, closely related to failure to control initial bleeding or early re-bleeding occurring in up to 30-40% of patients. The only approved drugs to arrest variceal bleeding are vasopressin and terlipressin. Treatment with terlipressin is preferable due to better efficacy, longer effects and less adverse effects compared to vasopressin.

Treatment with terlipressin has several drawbacks. The distribution half-life is 8 minutes, while the elimination half-life is 6 minutes. Consequently, terlipressin is typically administered by intermittent intravenous dosing schedule of approximately every 4-6 hours in doses of 1-2 mg per injection, until bleeding is under control. Duration of treatment can last up to 3 days.

FIG. 2 depicts the G-protein coupled V1 receptor system on vascular smooth muscle cell. Vascular smooth muscle cells (VSMCs) are the cellular components of the normal blood vessel wall that provide structural integrity and regulate the diameter by contracting and relaxing dynamically in response to vasoactive stimuli. V1 receptors are present on VSMCs. Upon activation of these receptors by vasopressin analogues, a G-protein-mediated release of calcium from intracellular stores within the sarcoplasmic reticulum occurs that subsequently activates membrane channels, allowing for the influx of extracellular calcium to further regulate calcium balance. Calcium concentration is the primary determinate of actin-myosin cross-bridge cycling and force of contraction in vascular smooth muscle; thus, increases in calcium concentration lead to systemic vasoconstriction.

Abnormal vasoconstriction (i.e., the narrowing of blood vessels from contraction of vascular smooth muscle tissue) is associated with pathologies such as hypertension, ischemia, and infarction. Esophageal varices (i.e., esophageal varix or esophageal varices) are dilated sub-mucosal veins in the lower portion of the esophagus often as a result of portal hypertension, typically due to cirrhosis. Persons having esophageal varices often develop bleeding.

Terlipressin is a cyclic dodecamer peptide (GGGCYFQNCPKG) drug, prescribed for bleeding esophageal varices, septic shock, hepatorenal syndrome and management of low blood pressure. It is an analogue of a naturally occurring hormone termed antidiuretic hormone (ADH) or vasopressin. This peptide interacts with multiple receptors in the body causing narrowing of blood vessels leading to an increase in blood pressure. It also regulates reabsorption of water in the renal medulla, preventing excessive loss of water in the urine. U.S. Pat. No. 9,090,064 describes terlipressin analogues and reports activity for several terlipressin amide and ester analogues.

In an embodiment, for example, a therapeutic polymer of this aspect comprises one or more monomers characterized by any of the formulas (FX5a), (FX5b), (FX5c), or (FX5d):

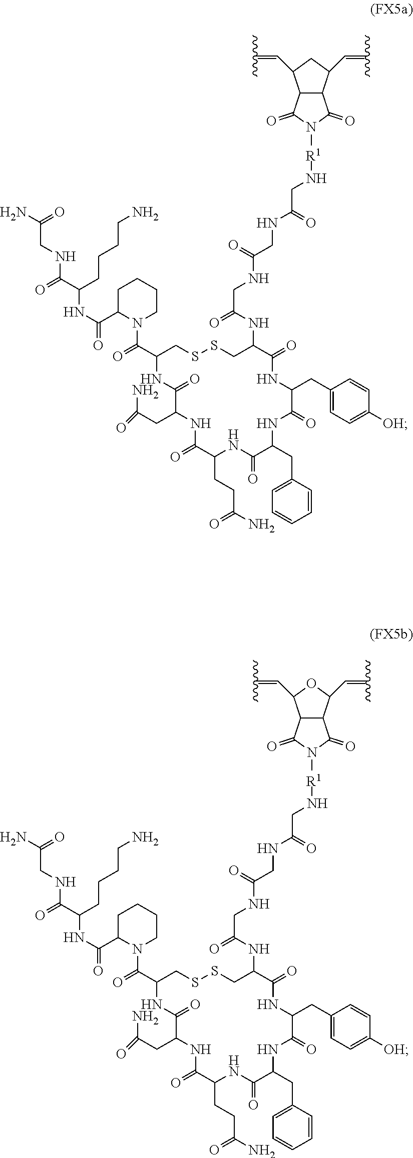

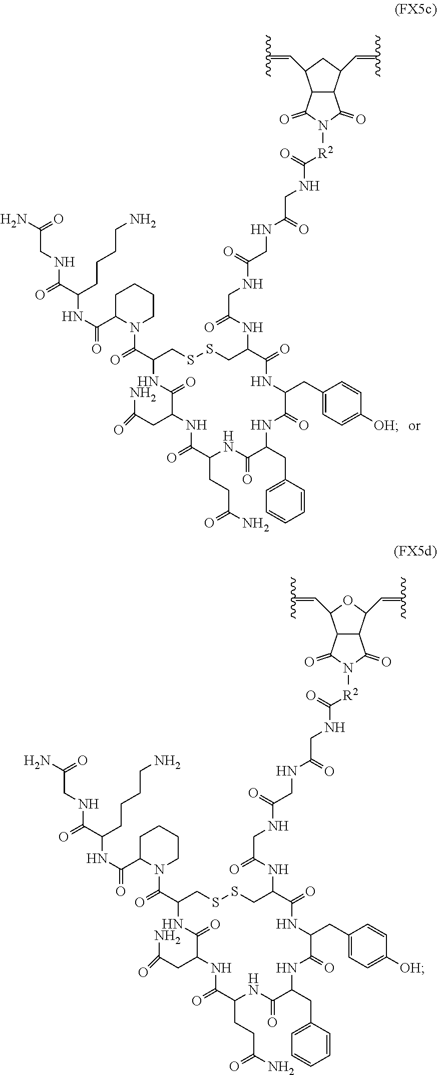

wherein each of R¹ and R² is independently substituted and/or unsubstituted C₁-C₂₀ alkylene, C₃-C₂₀ cycloalkylene; C₃-C₃₀ arylene or C₃-C₃₀ heteroarylene.

In an embodiment, for example, a therapeutic polymer of this aspect comprises one or more monomers characterized by any of the formulas (FX6a) or (FX6b):

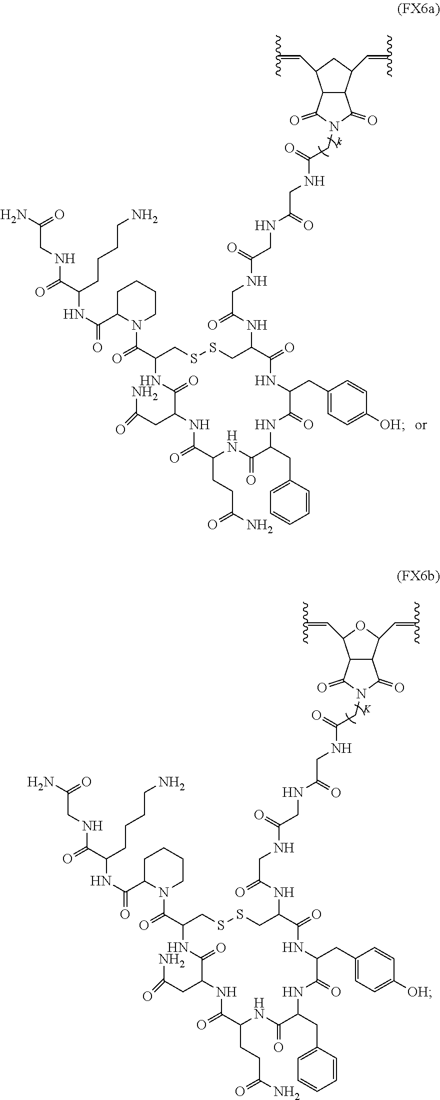

wherein each k is independently an integer selected from 1 to 20

In an embodiment, for example, a method of activating a vasopressin receptor comprising contacting the vasopressin receptor with the therapeutic polymer. The vasopressin receptor can be any suitable receptor subtype. Typically, the vasopressin receptor is selected from a V1a subtype, a V1b subtype, a V2 subtype, and combinations thereof.

In another embodiment, the invention provides a method of upregulating calcium concentration in a cell comprising contacting the cell with a polymer of this aspect. In a preferred embodiment, the cell is a vascular smooth muscle cell.

In yet another embodiment, the invention provides a method of treating a vasopressin receptor mediated disease in a subject in need thereof comprising administering to the subject an effective amount of a therapeutic polymer of this aspect. In a preferred embodiment, the vasopressin receptor mediated disease is selected from bleeding esophageal varices, septic shock, hepatorenal syndrome, low blood pressure, paracentesis-induced circulatory dysfunction, hyponatremia, and central diabetes insipidus. In a preferred embodiment, the invention provides a method treating varices in a subject in need thereof.

The following examples further illustrate the invention but, of course, should not be construed as in any way limiting its scope.

General Peptide Synthesis. Peptides (e.g. terlipressin) were synthesized using standard solid phase peptide synthesis (SPPS) procedures on an AAPPTec Focus XC automated synthesizer. Peptides were prepared on Rink amide MBHA resin. A typical SPPS procedure involved FMOC deprotection with 20% methylpiperidine in DMF (one 5 min deprotection followed by one 15 min deprotection), and 45 min amide couplings using 3.75 eq. of the FMOC-protected, and side chain-protected amino acid, 4 eq. of HBTU and 8 eq. of DIPEA. The cysteine building block used was protected with Acm protecting group at the side chain. Removal of Acm protecting group from the cysteine residues and formation of the disulfide bond was done on resin by mixing it with a thallium trifluoroacetate 1.2 eq dissolved in DMF (twice for 40 min).

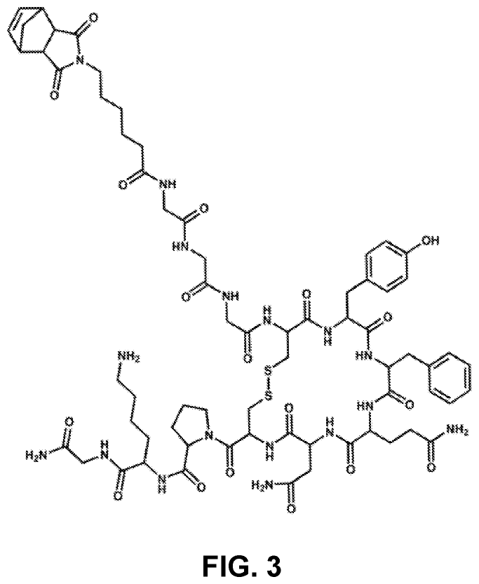

Norbornene conjugation. FIG. 3 depicts the structure of a terlipressin peptide monomer for using synthesizing brush polymers of certain embodiments of the invention. Peptide monomers (e.g., norbornene conjugates) were prepared by amide coupling to N-(hexanoic acid)-cis-5-norbornene-exo-dicarboximide at the N-terminus of the peptide. Following completion of the synthesis, peptides were cleaved from the resin by treatment with TFA/H₂O/TIPS in a 9.5:2.5:2.5 ratio for 2 h. The peptide monomers were then precipitated in cold ether and purified by RP-HPLC. The identity of the peptide monomers was confirmed by ESI-MS and purities were verified by observation of a single peak in analytical RP-HPLC chromatograms.

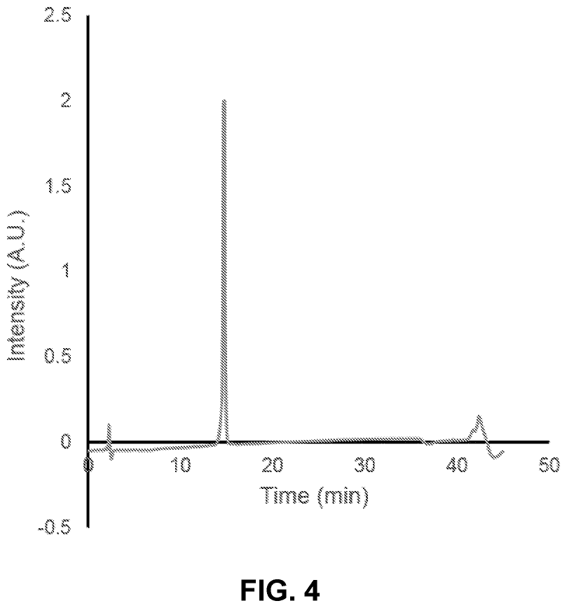

Peptide monomer characterization. The purity of the peptide monomers was verified by scale RP-HPLC, where a single peak in the chromatogram of a newly purified peptide monomer was taken as an indication of a pure material. RP-HPLC was performed on a Jupiter Proteo90A Phenomenex column (150×4.60 mm) equipped with a Hitachi-Elite™ LaChrom L2130 pump and a UV-Vis detector (Hitachi-Elite™ LaChrom L-2420) monitoring at 214 nm. FIG. 4 depicts the RP-HPLC chromatogram for a norbornene-terlipressin monomer prepared in accordance with an embodiment of the invention.

Peptide monomers were purified on a preparative-scale Jupiter Proteo90A Phenomenex column (2050×25.0 mm) using an Armen Spot Prep II System. In all cases, peptide monomers were purified and analyzed for purity using a gradient buffer system in which Buffer A is 0.1% TFA in water and Buffer B is 0.1% TFA in acetonitrile.

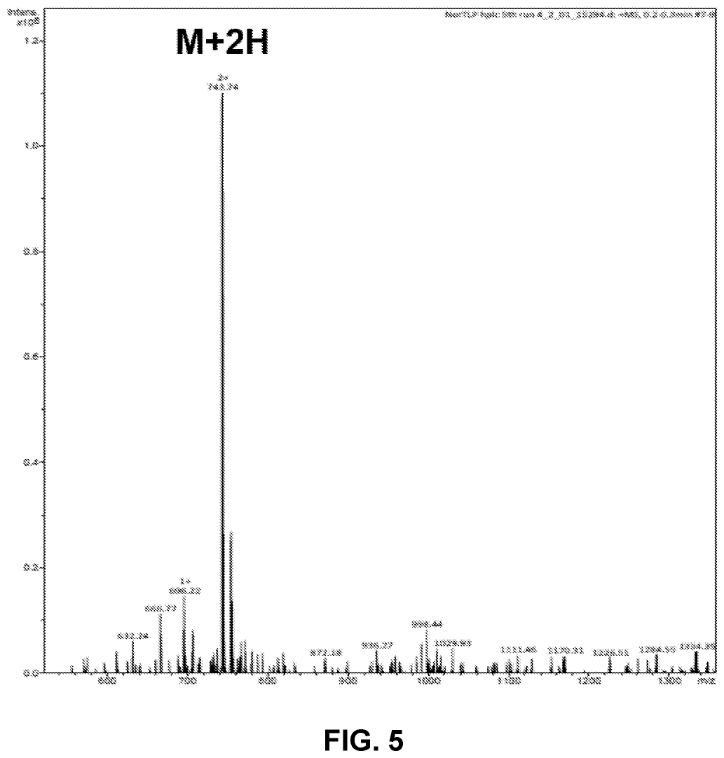

The identity of the peptide monomers was confirmed by ESI-MS. As shown in FIG. 5, the calculated mass for norbornene-terlipressin monomer (with two positive charges) was 744.34. The obtained mass was 743.74.

Polymerization kinetics. Polymerizations were carried out in a glovebox under N₂. A typical protocol used to generate a polymer with desired degree of polymerization by mixing the monomer and catalyst in respective ratio (e.g., 10:1) in dry DMF. For example, a DP of 10 involved mixing the monomer (0.0125 mmol, 10 equiv, 25 mM) with the catalyst (0.00125 mmol, 1 equiv, 2.5 mM) in dry DMF (0.5 mL).

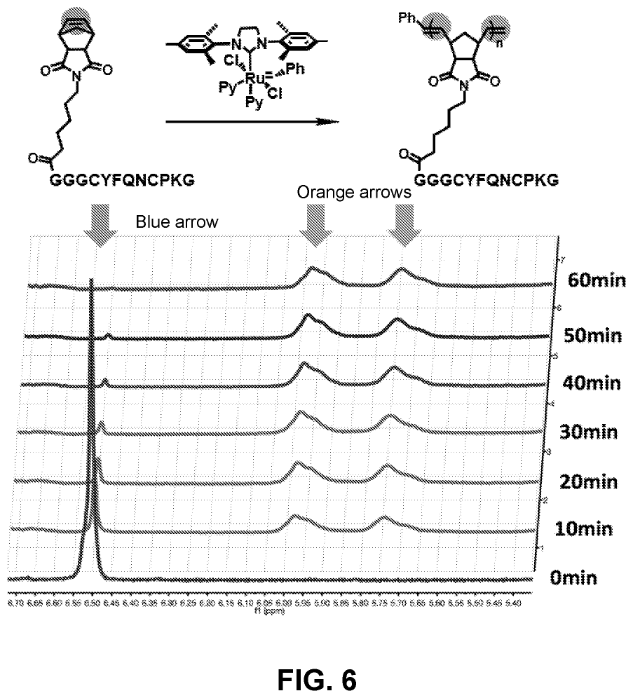

The polymerization reactions were monitored using ¹H NMR spectroscopy by measuring the consumption of the peptide monomer and to determine the time period required to reach completion. Termination was done with ethyl vinyl ether (10 eq) for 1 h at room temperature. FIG. 6. depicts the ¹H NMR time course spectra for the polymerization of norbornene-terlipressin monomers. The disappearance of the resonance at δ=6.50 ppm (blue arrow) corresponding to the olefin protons of the monomer and the coincident appearance of resonances at δ=5.7-6 ppm (orange arrows), which correspond to the cis and trans olefin protons of the polymer backbone.

The resulting polymers were directly characterized by SEC-MALS. The polymers were precipitated with cold ether and collected by centrifugation and dried.

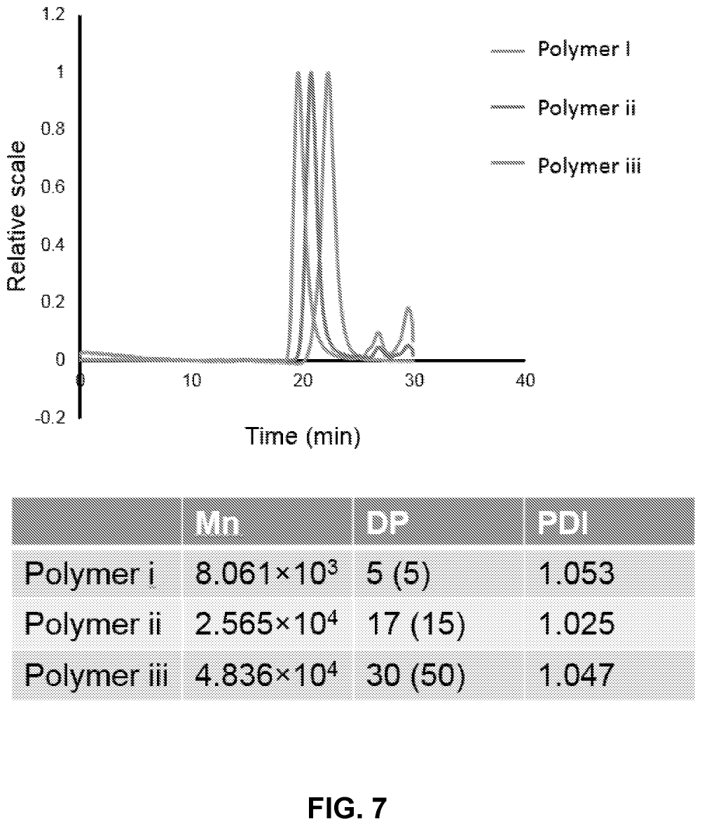

Light Scattering: Polymer dispersities (Mw/Mn) and molecular weights (Mn) were determined by size-exclusion chromatography coupled with multiangle light scattering (SEC-MALS). To this end, SEC was performed on a Phenomenex Phenogel 5u 10, 1K-75K, 300×7.80 mm column in series with a Phenomenex Phenogel 5u 10, 10K-1000K, 300×7.80 mm column, which ran with 0.05 M LiBr in DMF as the running buffer (flow rate of 0.75 mL/min) using a Shimadzu pump. The instrument was also equipped with a MALS detector (DAWN™ HELEOS™, Wyatt Technology) and a refractive index (RI) detector (Wyatt Optilab T-rEX detector). The entire SEC-MALS set-up was normalized to a 30K MW polystyrene standard. FIG. 7 provides SEC-MALS chromatograms for terlipressin polymers at a peptide m of ˜5.15 and 50 in DMF with 0.05 M LiBr.

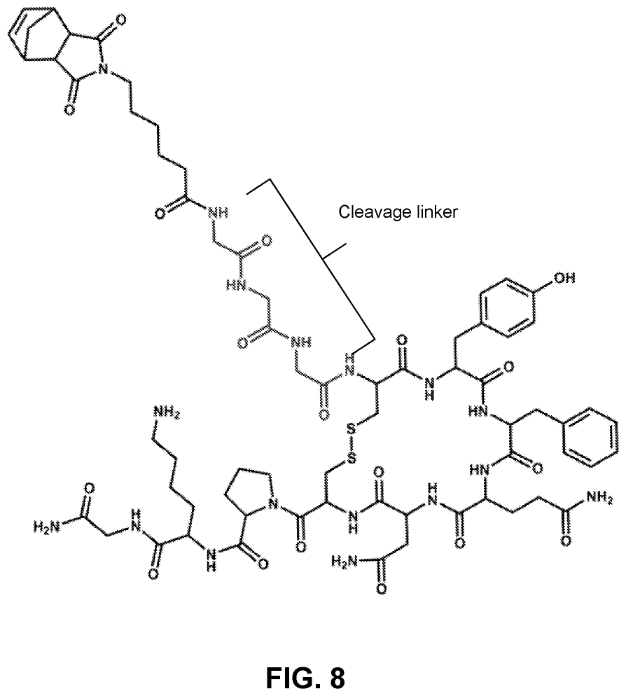

Enzymatic cleavage. Elastase was chosen as a model protease since it can cleave the three glycine fragment for easy recognition of cleavage product which is also similar to the natural initial cleavage product in serum. Remaining peptide percentage was measured by the ratio between the area under the curve at each time point and the area under the curve of the first time point. Approximately 50% peptide remain after 550 min following incubation with elastase. The identity of the correct cleavage product (CYFQNCPKG) was confirmed by ESI-MS. Calculated mass: 1056.23. Obtained mass: 1056.68. FIG. 8 provides the formula of a terlipressin peptide monomer indicated the structure of the GGG enzymatic cleavage site of a terlipressin peptide monomer.

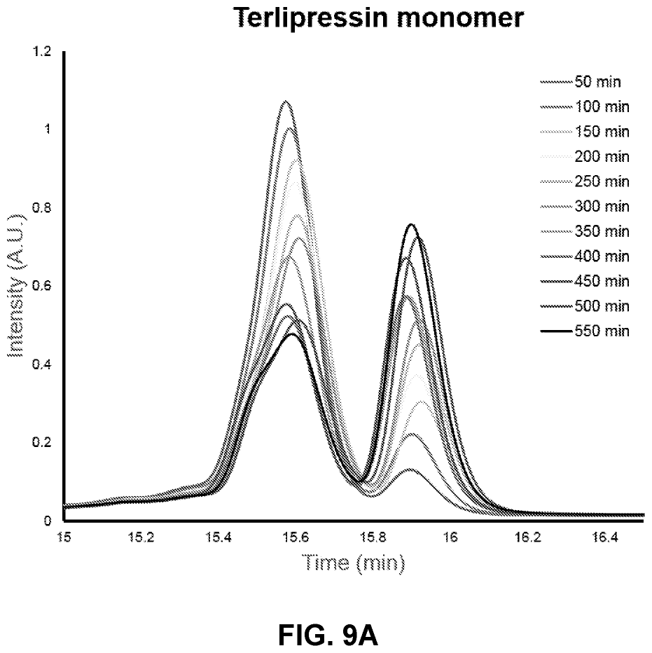

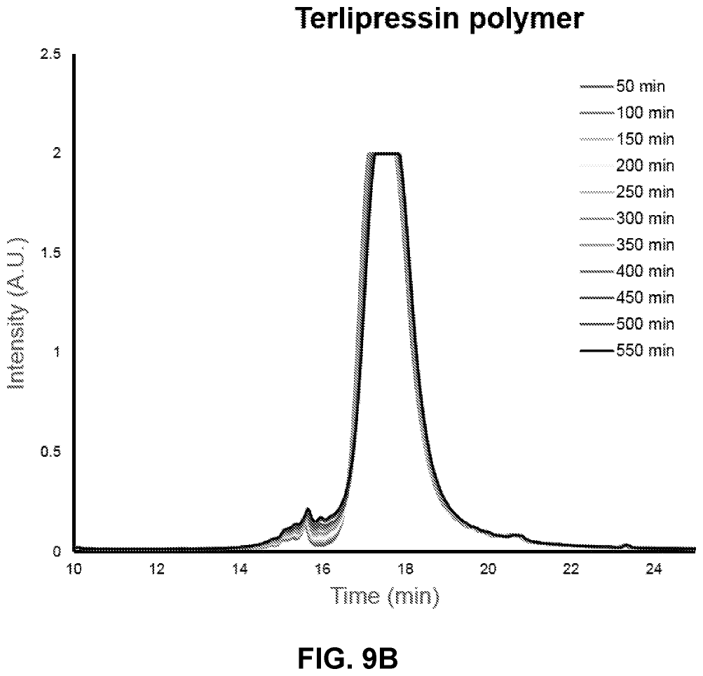

FIG. 9A-9B depicts the result of an enzymatic cleavage reaction. Twenty microliters (20 μL) of elastase (1 mM), 20 μL of monomer (FIG. 9A) or polymer (FIG. 9B) (2 mM), 20 μL Tris buffer (250 mM Tris pH 7.2, 1500 mM NaCl, 500 mM CaCl₂)) and 140 μL of water were added to a HPLC vial and loaded in the autosampler carousel and a sample immediately taken for injection at 1 min. Subsequently, the reaction was monitored via RP-HPLC (10-80% buffer B) with an injection every 50 minutes following the initial injection.

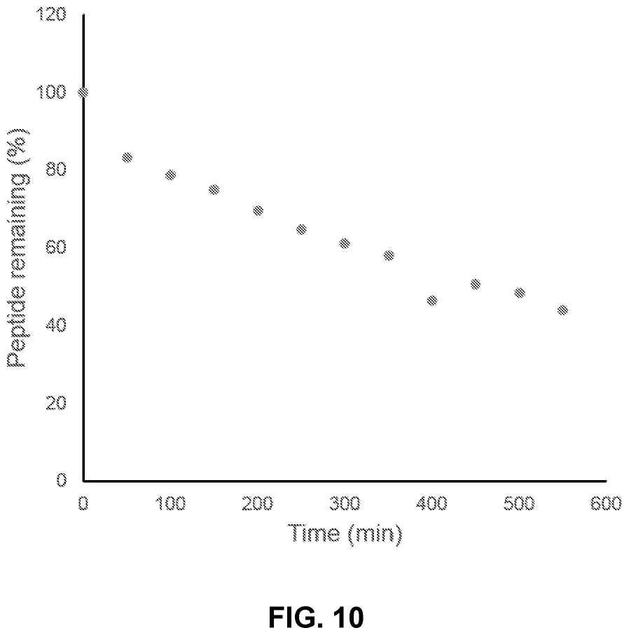

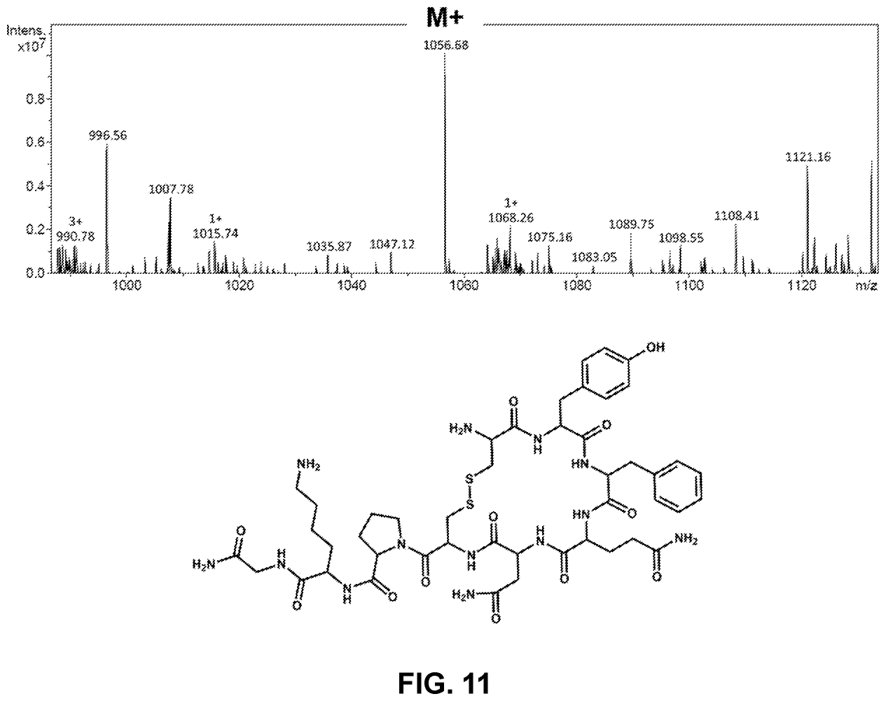

FIG. 10 depicts the ratio amount of peptide remaining after incubation with elastase. The ratio of the AUC at each time point relative to the first time point is plotted. Approximately 50% of the peptide remained after 550 min. FIG. 11 is structure of the enzymatic cleavage product and the mass spectrum (ESI-MS).

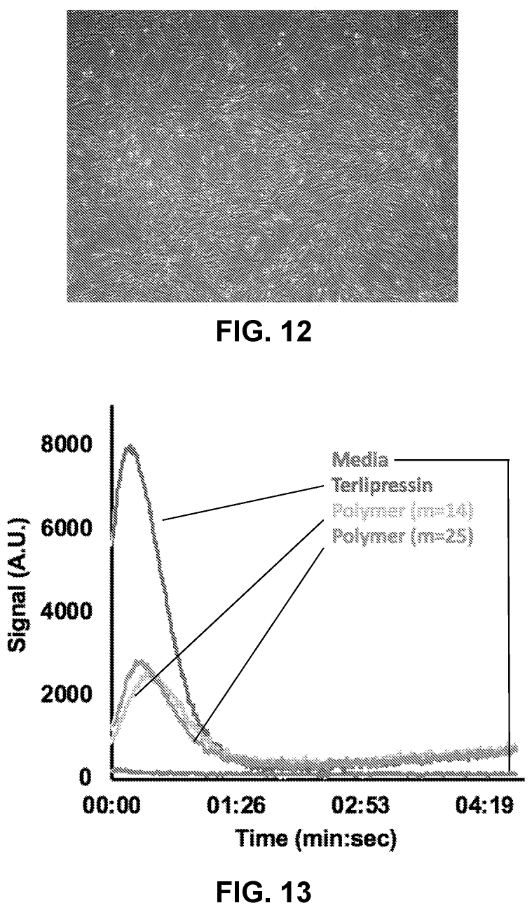

In Vitro Characterization. Primary human aortic smooth muscle cells (ATCC PCS-100-012 cell line) were cultured in vascular cell basal medium supplemented with rhFGF-basic 5 ng/mL, rh insulin 5 μg/mL, ascorbic acid 50 μg/mL, L-glutamine 10 mM, rh EGF 5 ng/mL, fetal bovine serum 5%. FIG. 12 depicts human aortic smooth muscle cells as used in the in vitro experiments described in the examples.

Calcium influx assays were performed using Fluo-4 direct calcium assay kit (ThermoFisher) on cells between passage number 3 to 6. To this end, cells were passaged and plated in a 96-well black tissue culture plate in a concentration of 10,000 cells per well following by incubation in 37° C. and 5% CO₂ for 48 hours. Cells were loaded with the dye according to the instructions of the kit and incubated for 1 h 37° C. and 5% CO₂. Then, terlipressin peptide monomers or polymers were added to the wells and fluorescence was measured immediately on a Perkin Elmer EnSpire™ 2300 plate reader. Signal was measured at 530 nm following excitation at 488 nm at 999 time points over a 5 min period. Non-fluorescent acetoxymethyl ester (Fluo-4 AM) is cleaved inside the cell. Fluorescence intensity increase upon binding Ca²⁺.

The results are set forth in FIG. 13 providing results for terlipressin, polymer (M=14), polymer (M=25) and media.

As is apparent from the results set forth in FIG. 13, the increase in signal following the addition of the polymers indicates that these materials can bind to and activate the V1 receptor on the cell surface of SMCs as the calcium levels observed in the smooth muscle cells increases significantly upon addition of the molecules to the cell media. The observed effect of the polymers in this assay is lower than that of the free peptide, but combined with the biological resistance of the polymers, they should provide relatively prolonged effect. In addition, the concentrations in this assay were calculated in respect to the peptide, putting the free peptide in advantage, as not all of the peptides unit on the polymer backbone are available to bind V1 receptors.

### Example 2—Peptide-Polymer Conjugate Biomaterials

Background

Existing methods to make peptide brush polymers include, for example, ring-opening metathesis polymerization, reversible addition-fragmentation transfer polymerization, and atom transfer radical polymerization methods. These methods have led to peptide brush polymers with different polymer backbones. However, stability or proteolytic resistance of these peptide brush polymers has not been sufficiently compared and evaluated. We for the first time directly compare the proteolytic resistance of peptide brush polymers with different polymer backbones. The results indicated that the selection of norbornene dicarboxyamide is important to render peptide brush polymers with resistance to proteinase for some clinical applications.

Therapeutic peptides are promising solutions to fight human diseases such as cancers and diabetes. However, their translations to clinical are hampered because peptides typically suffer from instability to proteinase and very short life time in vivo. To enhance their lifetime in plasma, peptide brush polymers were designed and synthesized via ring opening metathesis polymerization of peptides modified with a norbornene moiety. Specifically, two exo-norbornene monomers including (i) norbornene dicarboxyimide and (ii) norbornene amide were exploited. According to enzymatic digestion kinetics, peptide brush polymers made of norbornene dicarboxyimide monomers exhibited resistance to proteolytic digestions. On the other hand, peptide brush polymers featuring norbornene amide backbone exhibited lower stability to proteinase in comparison to their imide analogues. These results demonstrate the important role of polymer backbone in the proteolytic stability of peptide brush polymers under in vivo conditions.

Applications of the present peptide composition include therapeutic peptide delivery systems, high performance adhesive materials and protein delivery to cells

Innovation

This example demonstrates that structural variations of norbornene monomers play an important role in determining properties such as stability of peptide brush polyolefins. Importantly, the proteolytic stability of peptide brush polymers made of norbornene dicarboxyimide functional peptides outperformed their norbornene amide analogues under some conditions. This result highlights the structural effects of norbornene monomers and resulting polynorbornene backbones in stabilizing side chain peptides against enzymatic digestion.

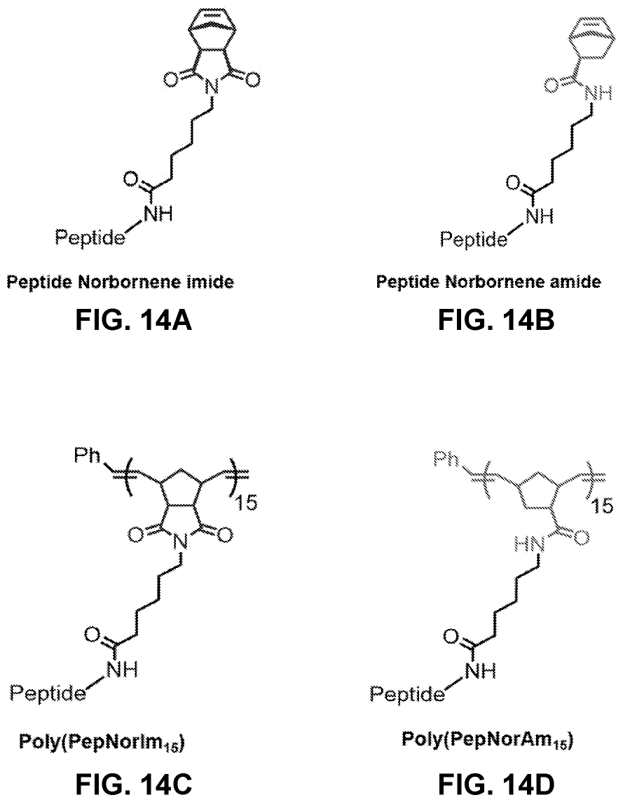

In this example, we systematically investigated key features of these materials including how the chemistry of the polymer backbone impacts the proteolytic stability of side chain peptides. Specifically, two peptide norbornene monomers including (i) norbornene dicarboxyimide and (ii) norbornene amide were exploited (FIGS. 14A-14D). According to enzymatic digestion kinetics, peptide brush polymers made of norbornene dicarboxyimide monomers exhibited resistance to proteolytic digestions. On the other hand, peptide brush polymers featuring norbornene amide backbone exhibited lower stability to proteinase in comparison to their imide analogues. These results demonstrated the important role of polymer backbone in dictating the proteolytic stability of peptide brush polymers.

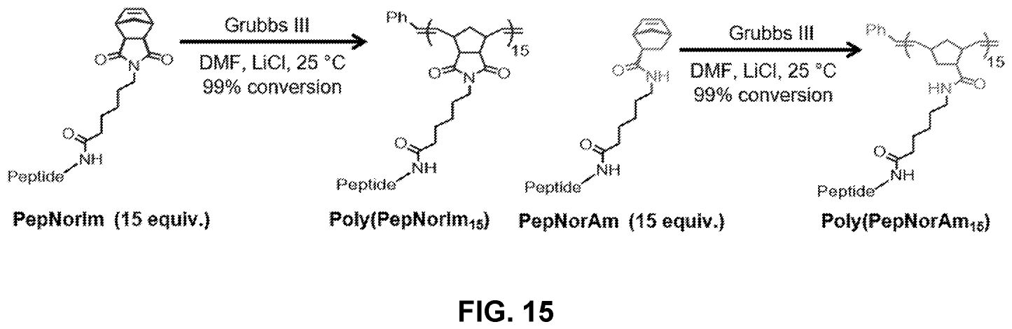

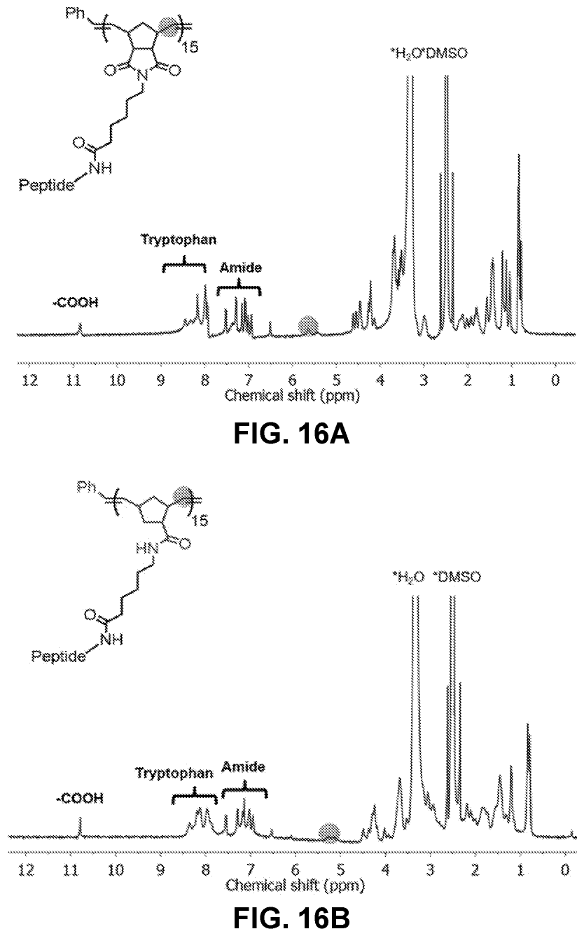

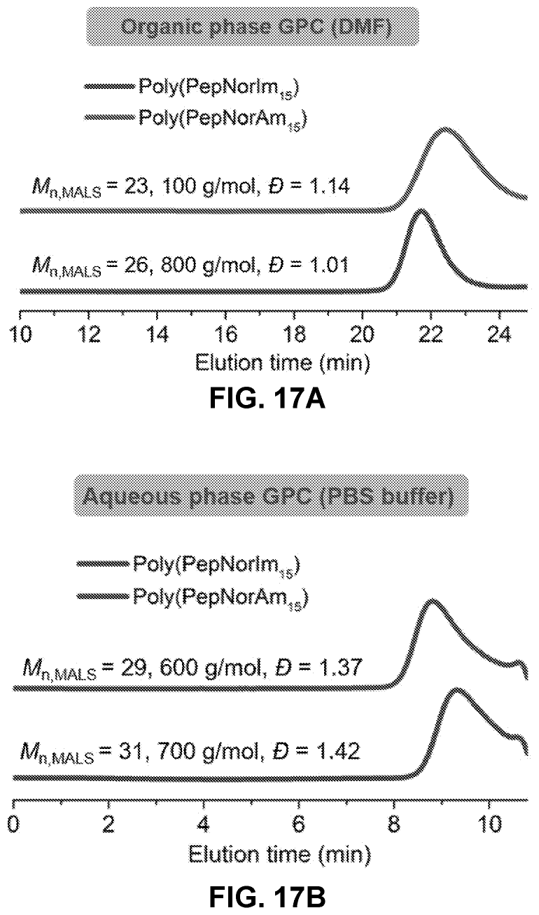

Well-defined peptide brush polymers bearing different types of polynorbornene backbones were synthesized by harnessing ring-opening metathesis polymerization (ROMP) (FIG. 15). We used an exemplary peptide sequence, GPLGLAGGWGERDGS, as an excellent substrate for matrix metalloproteinases (MMPs) and the bacterial protease, thermolysin. The chemical identity of peptide brush polymers featuring these two types of rigid polynorbornene backbones were confirmed by ¹H NMR spectra (FIGS. 16A-16B). To elucidate the aggregation state of polymers in aqueous environment, the molecular weights of peptide brush polymers were characterized by both organic phase (a good solvent) and aqueous phase gel permeation chromatography (GPC) (FIGS. 17A-17B). Notably, the molecular weights of peptide brush polymers in organic phase is in good agreement with those determined by aqueous phase GPC. This result indicated that peptide brush polymers predominantly exist as single chains rather than multi-chain aggregates in PBS buffer.

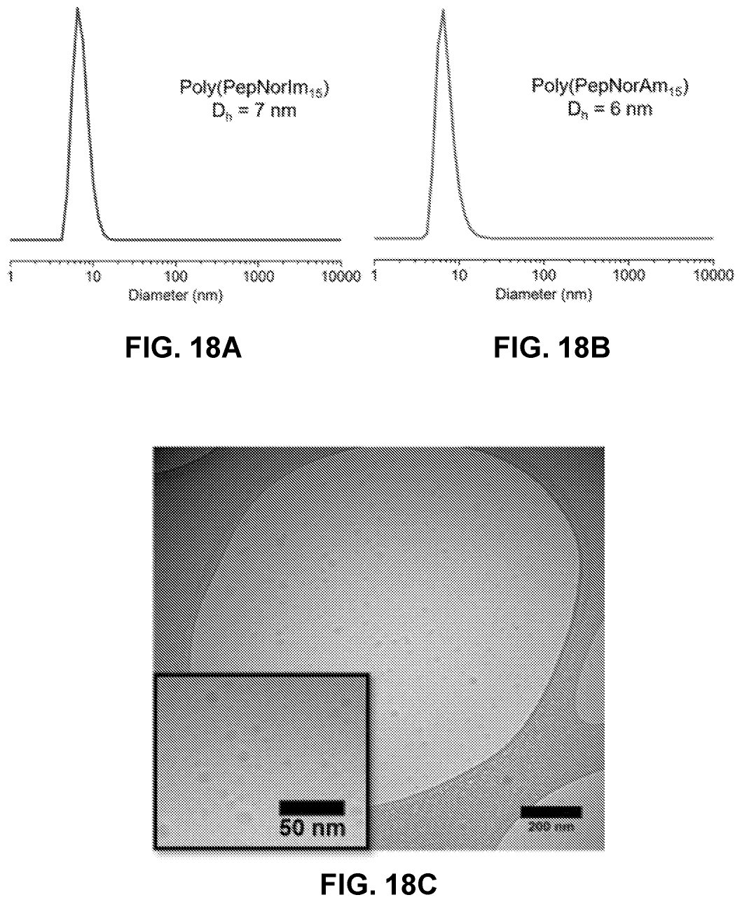

Furthermore, cryogenic TEM (cryoTEM) and dynamic light scattering (DLS) were utilized to determine the size and shape of resulting peptide brush polymers (FIGS. 18A-18C). According to DLS, both poly(PepNorIm15) and poly(PepNorAm15) are sub-10 nm in water. CryoTEM revealed a spherical morphology of peptide brush polymers. These results corroborate that the polymers are fully dispersed as individual chains in each case (FIGS. 18A-18C).

The efficacy or bioactivity of peptide brush polymers in some applications relies on several key factors including stability of peptide side-chains against proteases, receptor binding, cell penetration performance, and their interaction with targeted organelles after cellular uptake. Among those factors, peptide stability is the basis for achieving high efficacy of peptides in biological environments, through extending half-life following systemic administration (oral, intravenous, subcutaneous, etc.).

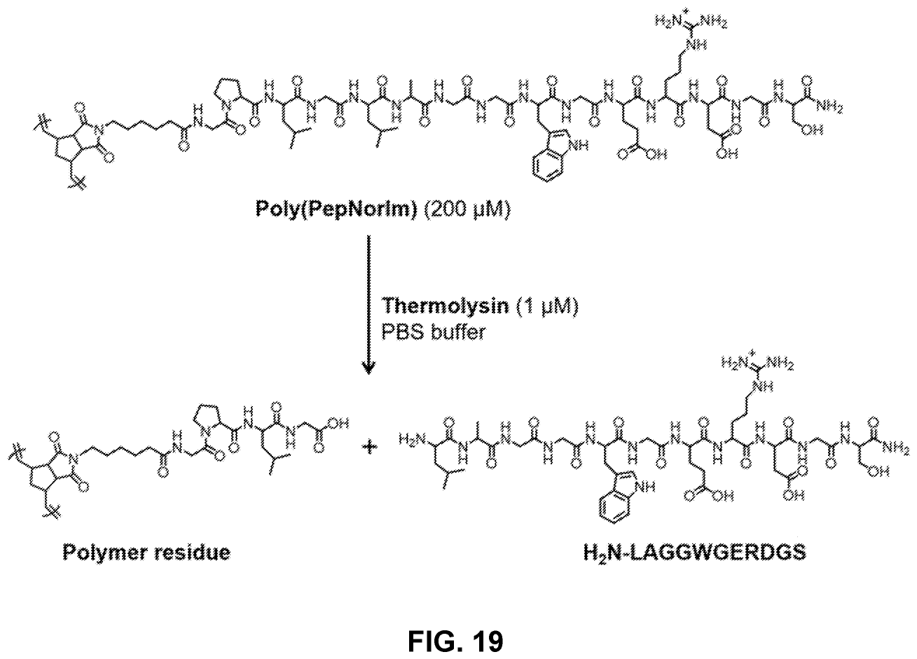

Towards these eventual applications, we evaluated the structural effects of peptide brush polymers on peptide stability. To discern the relationship between backbone structure and peptide stability against proteolysis, enzyme kinetics of each peptide brush polymer with different backbones were measured in PBS buffer with added protease (i.e., thermolysin). RP-HPLC was used to quantify the cleaved peptide fragments as a result of proteolysis to avoid using fluorescent labels that could alter the behavior of the enzymes further on modified substrates. In the study, a set of peptide brush polymers bearing pendent peptides (GPLGLAGGWGERDGS—SEQ ID NO: 19) were treated with thermolysin (FIG. 19), which can selectively cleave between glycine and leucine residues (i.e. amide at position 4 and 5) to generate a polymer residue and the peptide LAGGWGERDGS (SEQ ID NO: 17).

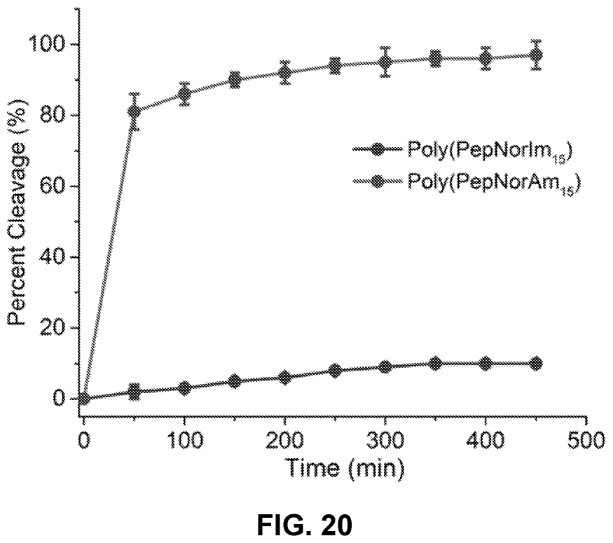

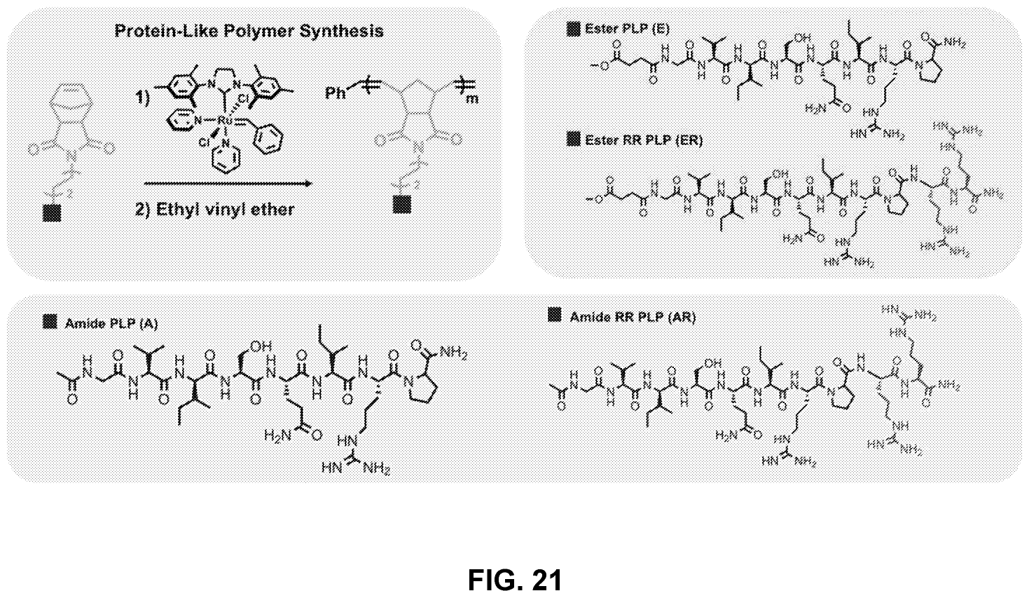

As demonstrated by HPLC results, poly(PepNorIm15) with the polynorbornene imide backbone demonstrated resistance to proteolysis, clearly outperforming the closely related amide-based backbone (FIG. 20). These data indicate significant variation possibly attributed to backbone-effects on the accessibility of peptides to proteases. We also note these backbone-effects with peptide substrates may be relevant to the human digestive system by conducting enzymatic digestion experiments using, for example, trypsin and pepsin. We will also examine the effect of DP on the proteolytic stability of brush peptides and elucidate rate constants and substrate affinities (kcat/KM) to fully describe the enzymology.

Instrumentation

¹H Nuclear Magnetic Resonance (1H NMR): ¹H NMR spectra were recorded on a Varian Inova spectrometer (500 MHz) in d₆-DMSO or CDCl₃. Chemical shifts are given in ppm downfield from tetramethylsilane TMS.

Analytical High-Performance Liquid Chromatography (HPLC): Analytical HPLC analysis of peptides was performed on a Jupiter 4 μm Proteo 90A Phenomenex column (150×4.60 mm) using a Hitachi-Elite LaChrom L-2130 pump equipped with UV-Vis detector (Hitachi-Elite LaChrom L2420).

Preparative HPLC: Armen Glider CPC preparatory HPLC was used to purify the peptides. The solvent system consists of (A) 0.1% TFA in water and (B) 0.1% TFA in acetonitrile.

Electrospray Ionization Mass Spectrometry (ESI-MS): ESI-MS spectra of peptides were collected using a Bruker Amazon-SL spectrometer configured with an ESI source in both negative and positive ionization mode.

Transmission Electron Microscope (TEM): Twenty microliters of sample were applied onto a 400 mesh carbon grids (Ted Pella, INC.). The grids were observed on a Hitachi HT 7700 microscope operating at 120 kV. The images were recorded with a slow-scan charge-coupled device (CCD) camera (Veleta 2k×2k).

Gel Permeation Chromatography (GPC): GPC measurements were performed on a set of Phenomenex Phenogel 5 μm, 1K-75K, 300×7.80 mm in series with a Phenomex Phenogel 5 μm, 10K-1000K, 300×7.80 mm columns with HPLC grade solvents as eluents: dimethylformamide (DMF) with 0.05M of LiBr at 60° C. Detection consisted of a Wyatt Optilab T-rEX refractive index detector operating at 658 nm and a Wyatt DAWN® HELEOS® II light scattering detector operating at 659 nm. Absolute molecular weights and polydispersities were calculated using the Wyatt ASTRA software with do/dc values determined by assuming 100% mass recovery during GPC analysis.

Dynamic Light Scattering (DLS): DLS analysis was conducted at room temperature on a Zetasizer Nano-ZS (Malvern). The laser for DLS was at a wavelength of 633 nm.

Experimental

Preparation of Peptide Monomers Via Solid-Phase Peptide Synthesis (SPPS)

Peptides were synthesized on Rink resin (0.67 mmol/g) using standard FMOC SPPS procedures on an AAPPTec Focus XC automated synthesizer. A typical SPPS procedure included deprotection of the N-terminal Fmoc group with 20% 4-methylpiperidine in DMF (1×20 min, followed by 1×5 min), and 30 min amide couplings (twice) using 3.0 equiv. of the Fmoc-protected amino acid, 2.9 equiv. of HBTU and 6 equiv. of DIPEA. After that, peptide monomers were prepared by amide coupling to Fmoc-6-aminohexanoic acid, followed by Fmoc deprotection and final amidation with acrylic acid (3 equiv.) in the presence of HBTU (2.9 equiv.), and DIPEA (6 equiv.).

Ring Opening Metathesis Polymerization

In a typical ROMP (synthesis of poly(PepNorIm15)), peptide (GPLGLAGGWGERDGS) norbornene monomer (30 mg, 15 equiv.) were dissolved in 650 μL of DMF. Then 50 μL (0.85 mg, 1.0 equiv.) of G-III stock solution (1.7 mg in 100 μL of DMF) was added into the reaction mixture. The reaction was left to stir and the monomer conversion was monitored by proton NMR. After polymerization, the reaction was quenched by adding 20 μL of ethyl vinyl ether. The polymer product was purified by precipitation into cold diethyl ether.

Thermolysin-Induced Cleavage Experiments

For enzyme-triggered cleavage experiments, the molar ratio of thermolysin to peptide was set to 1:200. Moreover, the temperature was set to 37° C. For example, poly(PepNorAm15) (1 mg, 0.60 μmol with respect to peptides, 200 equiv.) was dissolved in 1 ml of DPBS solution. Then thermolysin (0.1 mg, 3.0 nmol, 1 equiv.) was added into the polymer solution which was stirred in a preheated oil bath at 37° C.

### Example 3—Anti-Angiogenesis Treatment

Background

Neovascular age-related macular degeneration (nAMD) is the most common cause of irreversible vision loss in the developed world. nAMD is a global health problem with huge societal impact. After FDA approval of anti-VEGF antibody drugs in 2004, there has been significant advance in the treatment of patients with nAMD. The administration of these monoclonal antibodies has led to improvements in patient vision and overall quality of life. However, there remains a need for longer-lasting drugs and treatment options for the millions of patients who do not respond to anti-VEGF therapy. Our proposed research will develop long-lasting peptide-based drugs based on the alternative TSP1 pathway.

Choroidal neovascularization (CNV), marked by growth of new blood vessels, is the key pathology of nAMD. CNV causes bleeding and leakage of fluid into the retina, resulting in visual distortion, death of retinal cells, and central vision loss. Vascular endothelial growth factor (VEGF) is a key driver of angiogenesis during the development of CNV. Current therapy includes monthly injections of anti-VEGF medications into the vitreous cavity. Despite this advance, two key problems remain. First, monthly intravitreal injections of anti-VEGF antibodies are expensive. Second, 15% of patients demonstrate no improvement in visual acuity despite monthly anti-VEGF treatment. Therefore, a novel therapeutic pathway, independent of VEGF and more long-lasting than monthly injections is an unmet clinical need for nAMD patients. Our solution is (1) to use an anti-angiogenesis pathway based on Thrombospondin 1 (TSP1) independent of VEGF, and (2) to use a novel peptide packaging platform to create a long-lasting treatment.

TSP1 is an extracellular matrix protein that interacts with cell surface receptors and modulates several in vivo processes including angiogenesis. In immunohistochemical studies of post-mortem eyes, TSP1 is expressed in the RPE, BM, and CC, and its levels are reduced with age and further reduced in late stages of AMD, shifting the balance toward a pro-angiogenic milieu. As the first endogenous protein inhibitor of neovascularization to be discovered, TSP1 inhibits angiogenesis in a variety of different pathways: i) acting as a VEGF antagonist, ii) inducing cell apoptosis, and iii) modulation of cell proliferation and migration. Notably, TSP1 limits neovascularization in vascular endothelial cells by inducing receptor-mediated apoptosis. CD36 is a membrane protein that mediates the uptake of oxidized lipids and regulates the anti-angiogenic activity of TSP-1. CD36-TSP1 interaction down-regulates the VEGF receptor-2 and antagonizes VEGF function. Amongst the multifunctional domains within TSP1, the type-1 repeats domains bind to CD36. ABT898 is a synthetic peptide which mimics the TSP1 type-1 repeat. Synthetic peptides, such as ABT898, that mimic TSP-1 therapy, could inhibit angiogenesis. However, there are several challenges that limit the clinical use of therapeutic peptides. Peptides are prone to proteolysis, demonstrate rapid renal clearance, and are unable in many cases to cross cell membranes. To address these challenges, we highlight below a new innovative peptide packaging and delivery strategy to address challenges in nAMD therapies.

Innovation

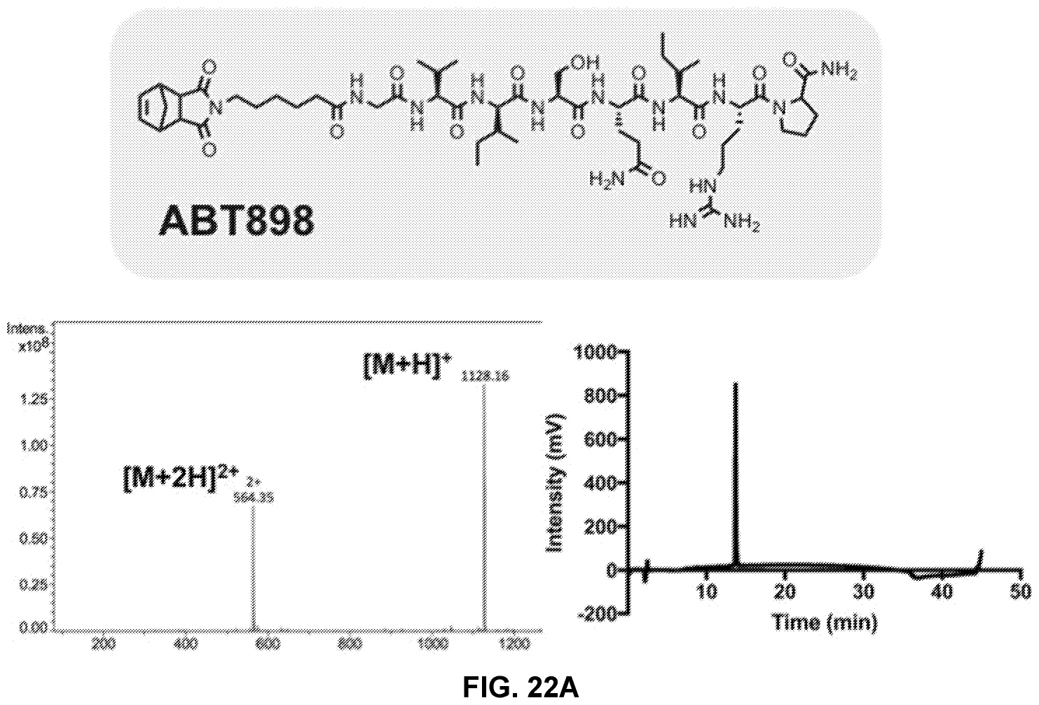

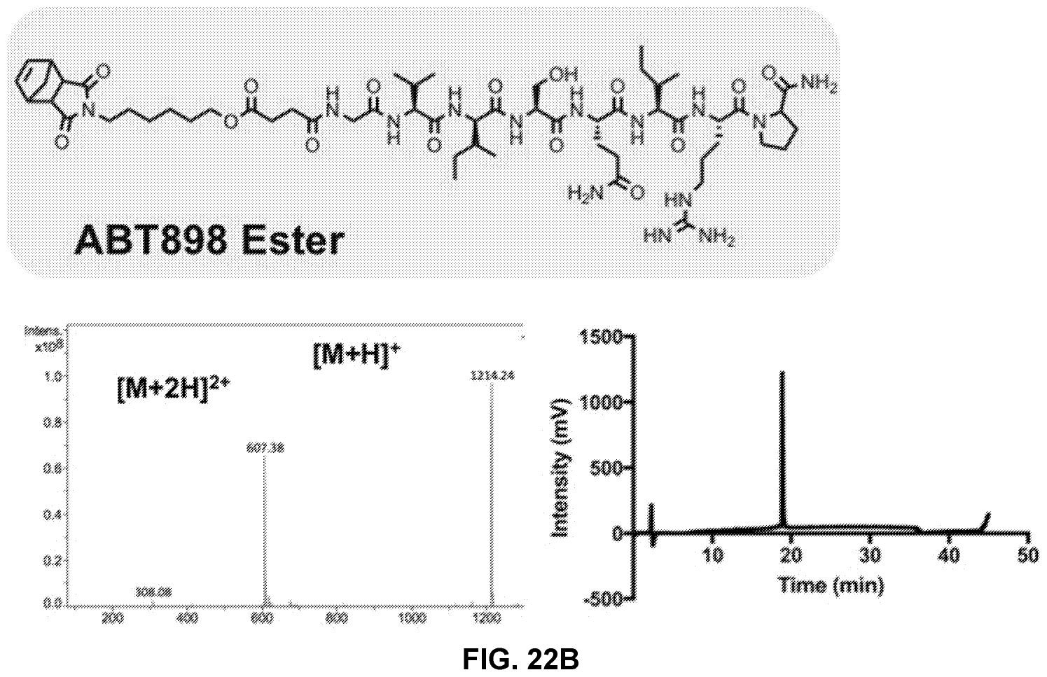

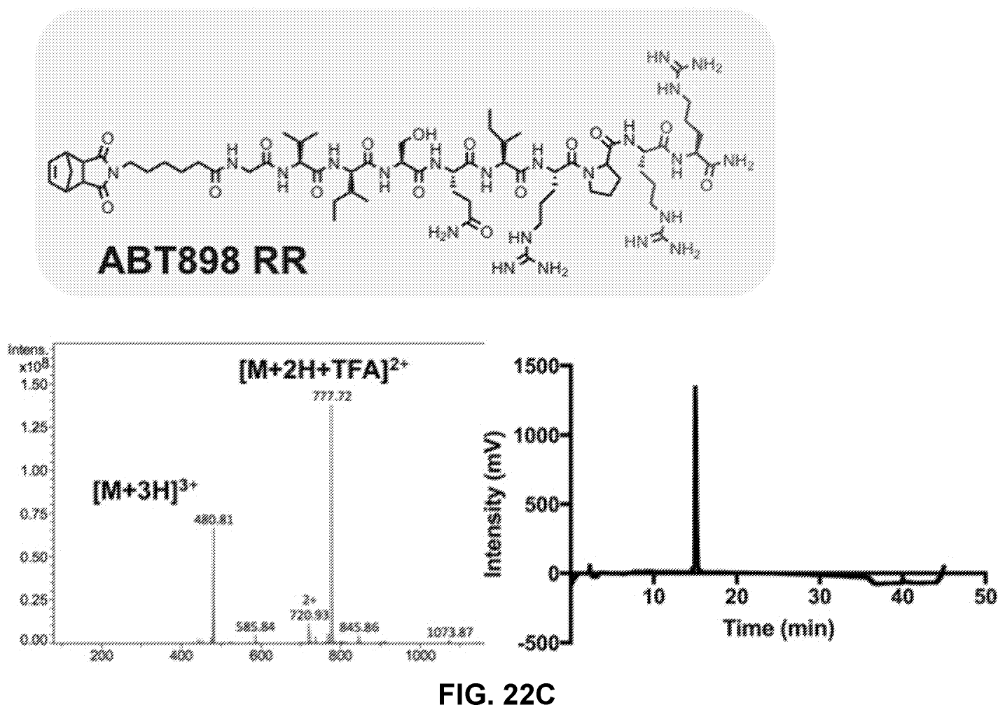

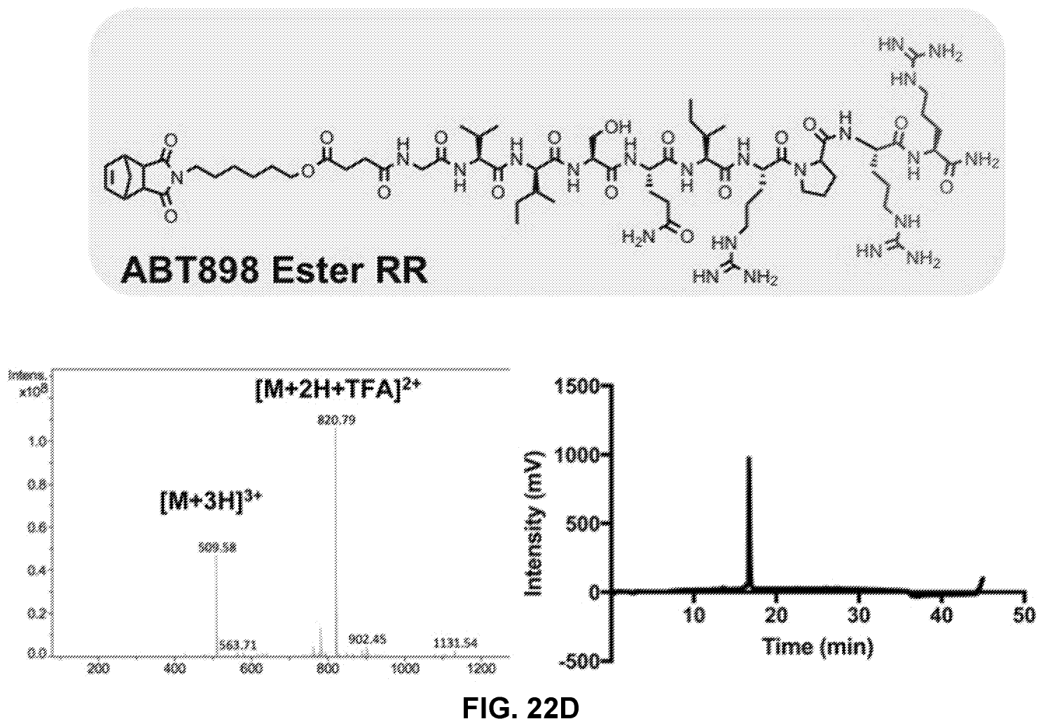

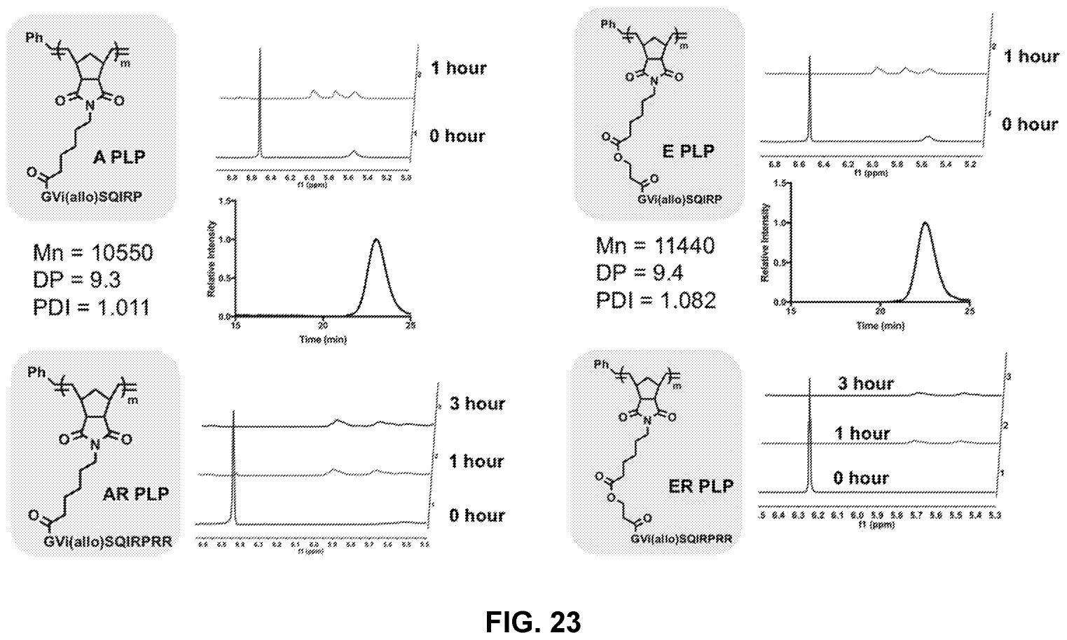

ABT898 PLP derivatives. As a first step towards the proposed work, we synthesized four norbornene modified ABT898 (GVi(allo)SQIRP—SEQ ID NO:4) sequences with different linkers and additional arginine amino acids (FIG. 21). Characterization data (e.g., ESI-MS, HPLC trace, and NMR data) for the four norbornene modified ABT898 (GVi(allo)SQIRP—SEQ ID NO:4) sequences are shown in FIGS. 22A-22D and 23. Different linkers were incorporated to compare the bioactivities of the cleavable (Ester PLP and Ester RR PLP) and non-cleavable (Amide PLP and Amide RR PLP) PLPs. Cell penetrating peptides, e.g. TAT sequences, are short sequences that are rich in arginine residues and aid the cellular penetration of cargo via endocytosis. Therefore, to enhance the cellular penetration capacity of the PLPs we modified the C-terminus with arginine moieties (Ester RR PLP and Amide RR PLP). These norbornene functionalized ABT898 peptide sequences were then subjected to standard ring opening metathesis polymerization (ROMP) conditions to yield four different ABT898 PLPs (FIG. 1).

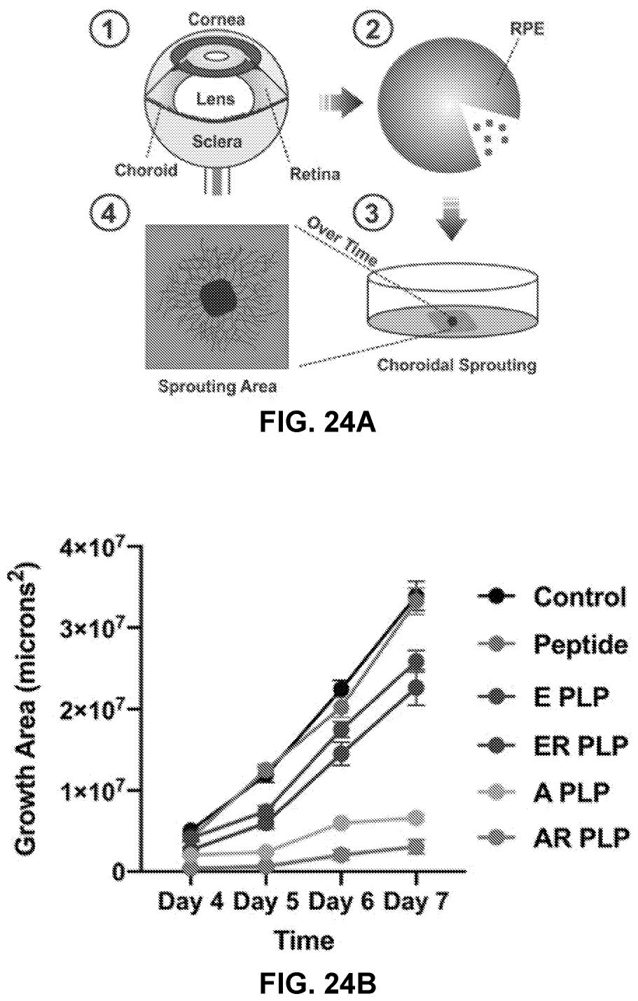

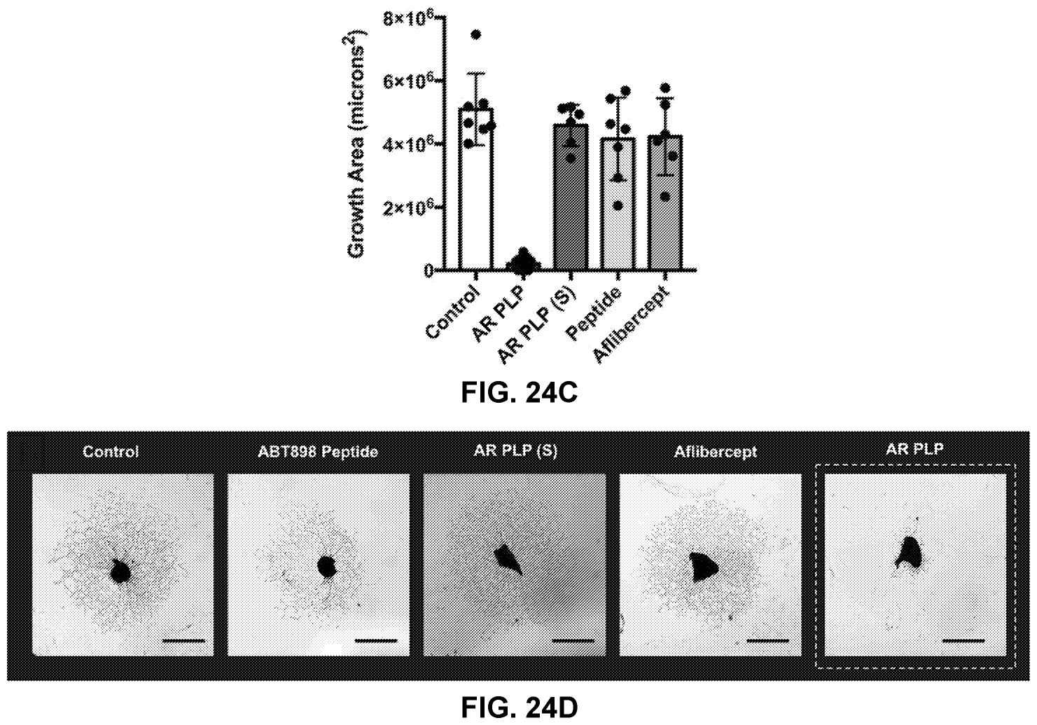

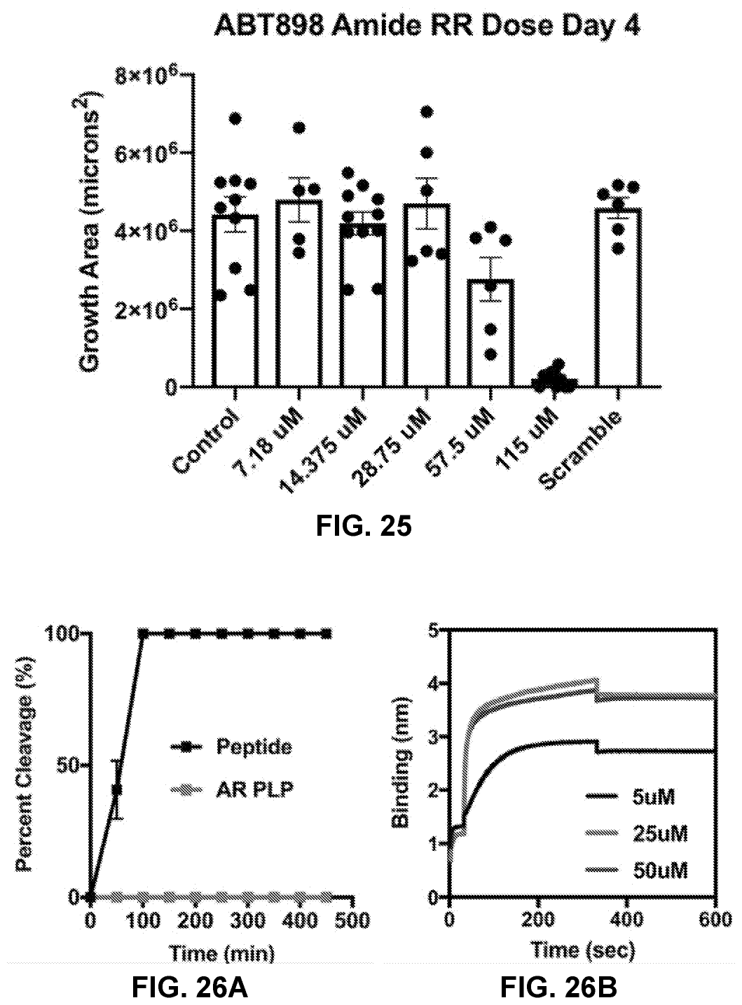

Ex vivo choroidal sprouting assay. As a first step towards determining the anti-angiogenic properties of the ABT898 PLPs, we performed an ex vivo choroidal sprouting assay. Briefly, eyes are enucleated, dissection is performed to remove the cornea, iris, lens, retina, and vitreous. The peripheral RPE-choroid-sclera complex is plated in Matrigel, angiogenesis develops spontaneously over Day 4-Day 7, and can be quantitated using reverse phase brightfield microscopy (FIG. 24A). ABT898 peptide had no effect upon choroidal angiogenesis (FIGS. 24B and 24C). Alternatively, ABT898 Ester and Amide PLPs, which are not hydrolysable, reduced angiogenesis by 24% and 81%, respectively. Cell penetrating peptides that are rich in arginine residues, aid the cellular penetration of cargo via endocytosis. In order to enhance the cellular penetration capacity and the solubility of the PLPs we modified the C-terminus with arginine moieties. Furthermore, addition of the RR sequence to increase solubility and cell penetration, further decreased angiogenesis by 33% (Ester RR) and 91% (Amide RR), respectively. This indicates that the non-cleavable, arginine modified ABT898 PLP has improved bioactivity compared to the original peptides. Without wishing to be bound by any particular theory, we believe this is because the peptide alone is degraded in the Matrigel milieu. In addition, ABT898 Amide RR PLP was far more anti-angiogenic than scrambled PLP and commercial aflibercept (FIG. 24D), indicating that the specific peptide sequence plays a crucial role in determining the extent of anti-angiogenic function. In addition, we performed a dose dependent analysis of growth area at Day 4 for treatment with ABT898 amide RR PLP, and the results are shown in FIG. 25. These results demonstrate that at dosages of 57.5 μM and 115 μM, the growth area is significantly reduced.

Enzyme Degradation Kinetics. An important feature of the PLP platform is that the globular structure of peptides arranged on a polymeric scaffold impart high proteolytic resistance. We performed an enzyme resistance assay to confirm that the PLP is resistant to proteolysis as compared to the peptide alone. Both peptide and PLP stock solutions (200 μM) were treated with thermolysin enzyme (0.1 μM) and their degradation profiles were monitored using high performance liquid chromatography (HPLC). We observed rapid degradation of the peptide to yield a fragment peak over time (FIG. 26A). However, the PLP peak does not change over time. We calculated the area under the peak at each time point to construct the percent cleaved and plotted it against time for both peptide and PLP (FIG. 26A).

Bio-Layer Interferometry Binding Assay. Quantifying the ligand-receptor binding interaction is an important step in the development of these PLPs. Similar to surface plasmon resonance, bio-layer interferometry (BLI) is an optical method used to measure the affinity of ligand-receptor binding. BLI has three key advantages over existing techniques: i) samples can be easily recovered, ii) it uses less sample, and iii) it is high throughput. We proceeded to study the interaction of CD36 receptor with ABT898 amide RR PLP (degree of polymerization, m=19). First, sensor labelled with anti-histidine tag was equilibrated with solvent before associating it with commercially available histidine-tag labelled CD36 receptor. Aqueous solutions of amide ABT898 amide RR PLP at various concentrations were assayed to study their binding with the CD36 receptor (FIG. 26B). As a negative control, we measured the binding of a scrambled ABT898 sequence PLP. We recorded a dissociation constant in the 10⁻¹² M⁻¹ range for ABT898 amide RR PLP versus 10⁻⁸ M⁻¹ for the scrambled analog. ABT510, another TSP-1 peptide sequence, has been shown to have nanomolar affinity for human microvascular cells. These data indicate that the ABT898 amide RR PLP have a strong affinity for CD36 receptor.

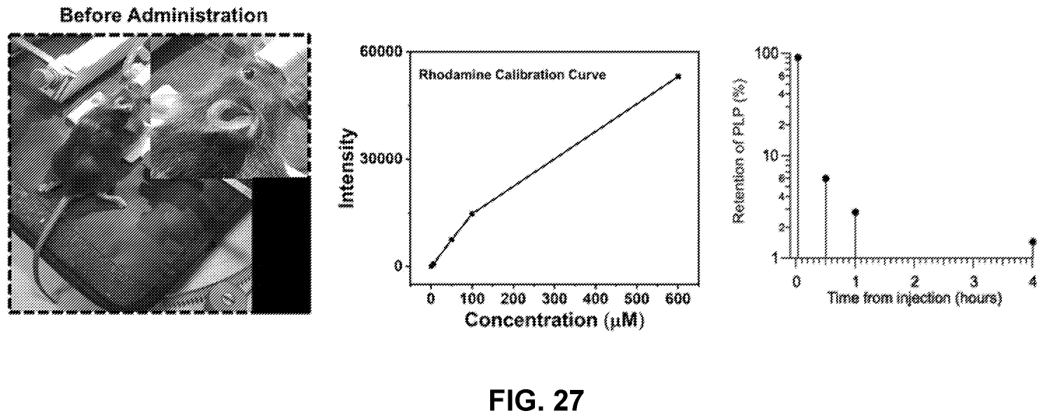

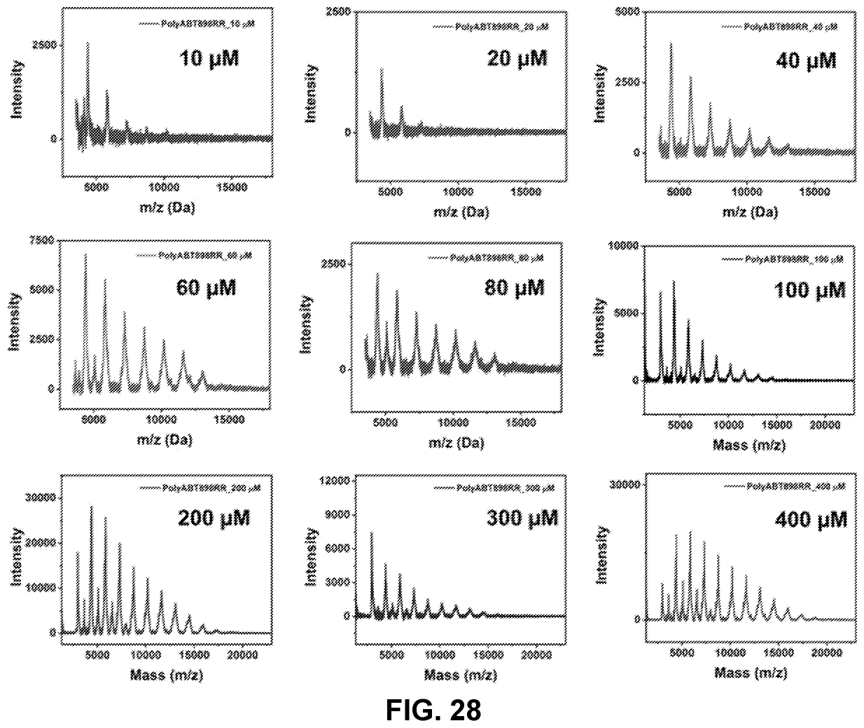

In vivo Injection Study. To determine the viability of delivering a PLP to the eye, a rhodamine labeled AR PLP was injected intraocularly, and the pharmokinetic profile was monitored, as shown in FIG. 27. In order to determine the limit of detection of the polymer in the eye. MALDI characterization of AR PLP at different concentrations was performed and the results are set forth in FIG. 28. As demonstrated by the graph showing retention of PLPs set forth in FIG. 27, the polymer is cleared from the vitreous fluid within 4 hours after injection.

Anti-VEGF Anti-Angiogenic Peptides and Corresponding PLPs. In addition, we employed our novel peptide-packaging technology to graft anti-VEGF peptides into polymers in order to improve their half-life, resistance to enzymatic degradation, and retention.

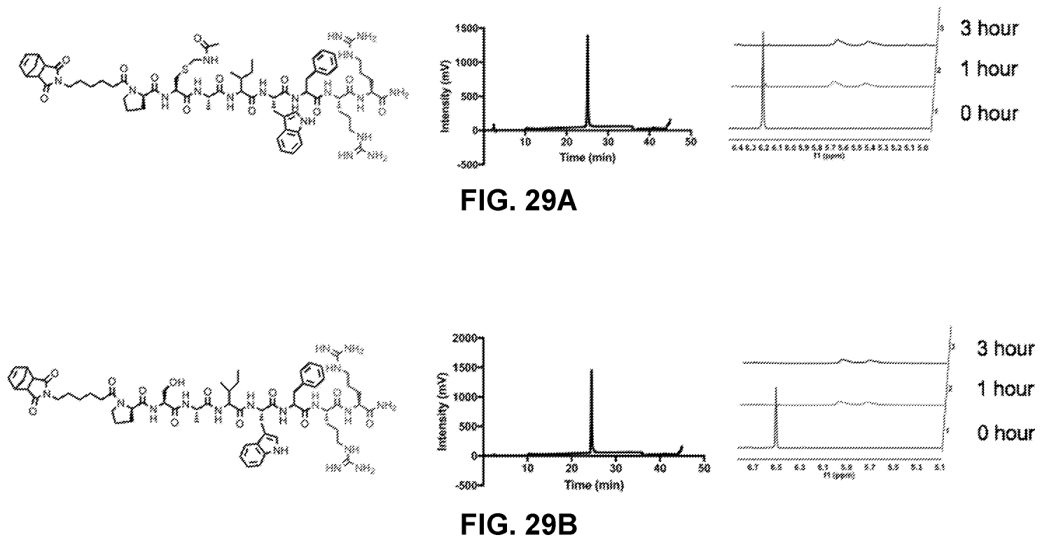

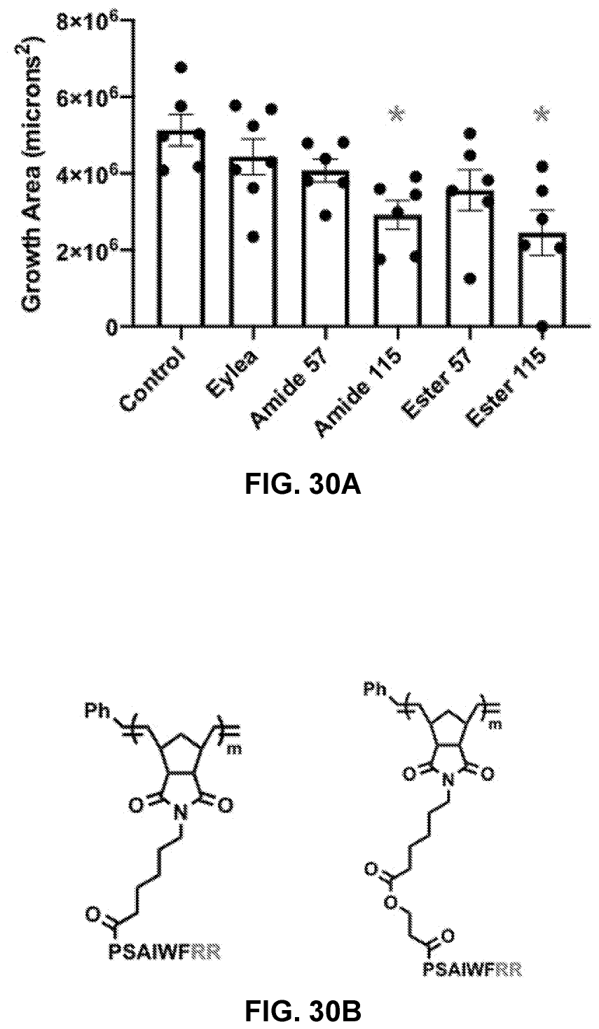

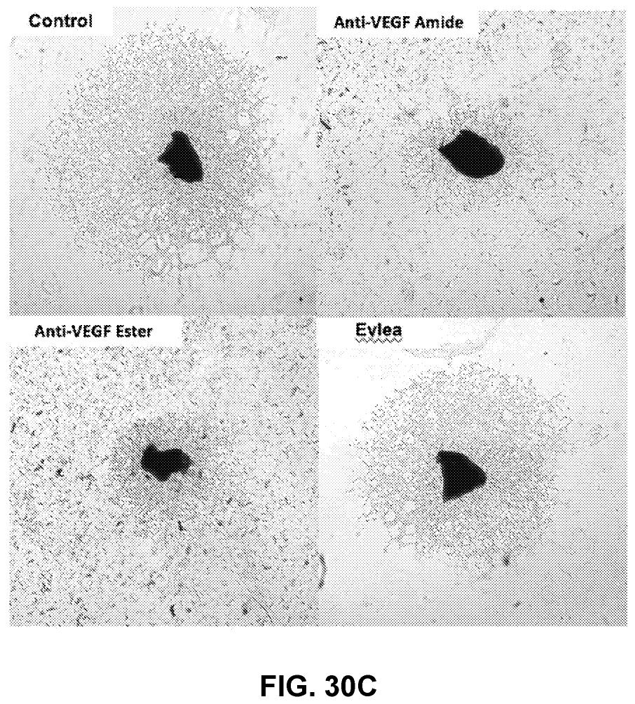

As a first step towards synthesis of anti-VEGF PLPs, we synthesized four different norbornene modified anti-VEGF (PCAIWF—SEQ ID NO:3 & PCAIWF—SEQ ID NO:18) sequences containing both an amide linkage and an ester linkage as set forth in FIGS. 29A, 29B, and 30B. The cysteine versions of the norbornene monomer were protected with ACM protected before polymerization and thereafter deprotected. We also synthesized analogous serine versions in order to test their activity. Cell penetrating peptides, e.g. TAT sequence, are short sequences that are rich in arginine residues and aid the cellular penetration of cargo via endocytosis. Therefore, to enhance the cellular penetration capacity of the PLPs we modified the C-terminus with arginine moieties. These norbornene functionalized anti-VEGF peptide sequences were then subjected to standard ring opening metathesis polymerization conditions to yield four different anti-VEGF PLPs (FIG. 30B).

Ex Vivo Choroidal Sprouting Assay: Anti-VEGF PLPs. We performed an ex vivo choroidal sprouting assay to determine the anti-angiogenic properties of the anti-VEGF PLPs at concentrations of 57 μM and 115 μM (FIG. 30A). After measuring the sprouting growth area, we observed that the anti-VEGF polymers inhibit the growth of new blood vessels. As demonstrated by the growth area shown in FIG. 30A and the microscopic images shown in FIG. 30C, the anti-angiogenic activity of the PLPs is better compared to commercially available drug Eylea (Aflibercept). Aflibercept is a recombinant fusion protein composed of VEGF binding portions from the transmembrane domains of human VEGF receptors 1 and 2, which are fused to Fc portion of human immunoglobulin. The molecular weight of Aflibercept is ˜96 kDa, a very large number compared to our anti-VEGF PLPs (˜15 kDa). Without wishing to be bound by any particular theory, we believe that large PLPs with molecular weights analogous to large monoclonal antibodies and fusion proteins will serve not only as long acting therapies but also more efficacious alternatives.

### Example 4—Delivery of Tumor-Associated Antigen gp100

Background

The utilization of tumor antigens, which are antigenic peptides expressed in cancer cells as a result of random somatic mutations, for the development of patient-specific therapeutic cancer vaccines has been an area of intense interest. Cancer vaccines based on peptide tumor antigens have been shown to be an effective treatment option for various types of cancers. However, challenges in the delivery and protection of these peptides from degradation and clearance to efficient display, have severely limited their clinical utility. This proposal seeks to address these challenges by utilizing a novel peptide delivery system that allows for the protected delivery of tumor antigens, either alone or in proportion-controlled combinations with multiple different antigens and immunomodulatory compounds.

Neoantigen-based cancer vaccines have shown significant therapeutic potential in early-phase clinical trials. However, several significant hurdles remain before immunogenic tumor antigens can be effectively used to induce robust anticancer immunity. First, effective targeted delivery of tumor antigens to professional antigen-presenting cells in a favorable immune milieu has remained elusive. Second, peptide tumor antigens are susceptible to proteolytic cleavage before being able to elicit an immune response. This example aims to demonstrate that the polymers of the invention provide a new means for enhancing peptide delivery to immune cells that allows for the sustained delivery of tumor antigens in conjunction with immunomodulatory compounds as rationally designed cancer vaccines.

Innovation

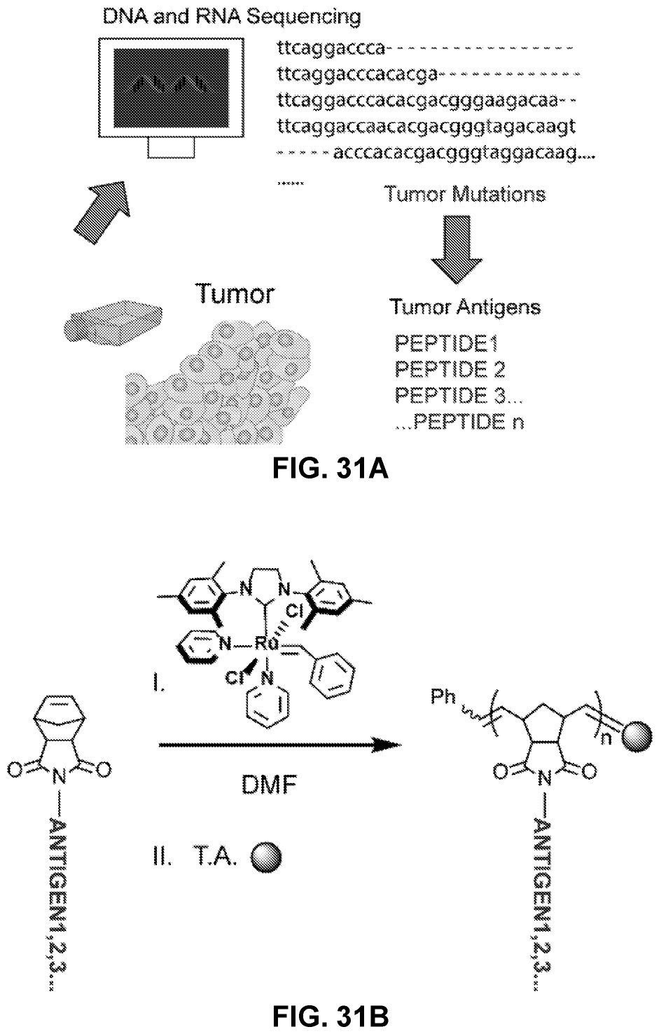

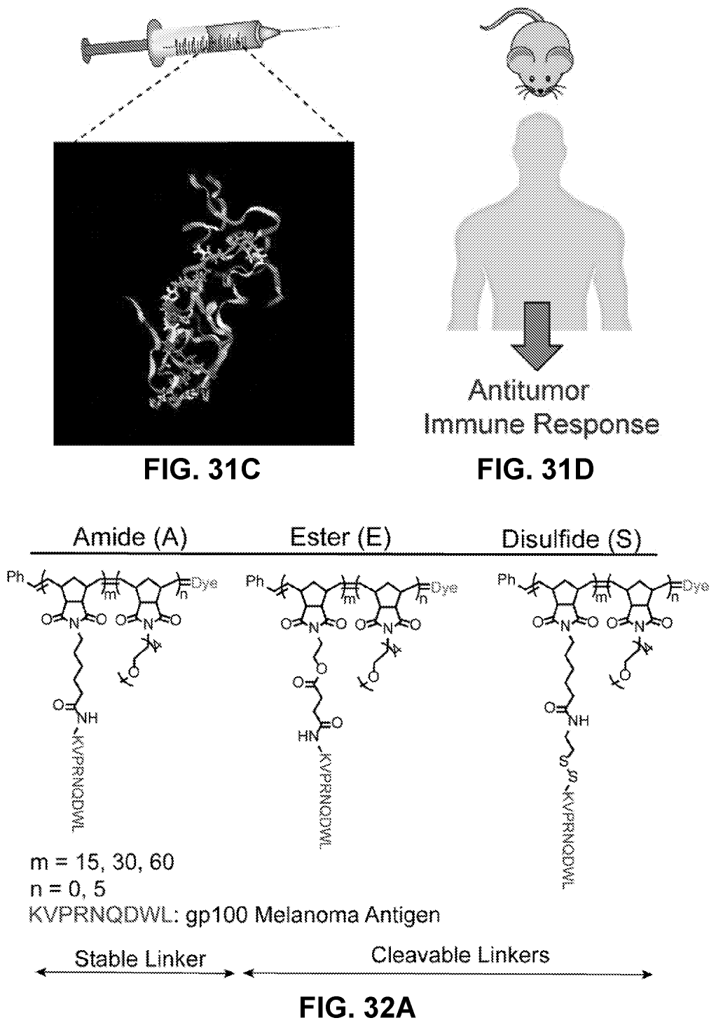

This proposal utilizes a new platform technology referred to as the Protein-Like Polymer (PLP) due to its globular structure, with peptide side chains assembled around a hydrophobic polymer core. The bioactive peptide side chains compose the vast majority of the molecular weight of PLPs, with the linking polymer backbone consisting of a small percentage of the overall construct. These single-polymer chain nanoparticles display bioactive amino acids in a precisely controlled manner, allowing for delivery of peptide antigens alone or in combination with other immunomodulatory compounds to avoid immune tolerance. See FIGS. 31A-31D for a process flow diagram. The novelty of this work stems from the ability to protect active peptide mixtures from proteolysis, vastly improving cellular uptake and bioavailability while maintaining strong bioactivity and a simple synthetic scheme. Our studies have shown that polymerization of monomers consisting of natural L-amino acids results in PLPs that resist enzymatic degradation entirely for several hours to days, whereas the same unpolymerized peptides undergo proteolysis within minutes under the same conditions. This phenomenon is driven by the globular nature of PLPs formed via the hydrophobic effect, spatially protecting proteolytic enzyme recognition. This is a model supported by in silico simulations and experiment. Importantly, despite their dense packing, PLP peptides maintain bioactivity and can be readily taken up by cells where they can act on intracellular targets. These characteristics of PLPs are agnostic to amino acid composition, which can be modified to incorporate a wide variety of peptide sequences including neoantigens identified from patient samples (FIG. 31A). Mixtures of dissimilar peptide sequences can be incorporated as polymer side chains (FIG. 31B) with immunomodulatory compounds attached as terminating agents (TA) or mixed in as additional side chains. These aforementioned characteristics (resistance to proteolysis, maintenance of bioactivity, and sequence-specific control of side chain composition) make PLPs an ideal platform for the rational design of cancer vaccines.

We have demonstrated the feasibility of this proposed approach using a model tumor-associated antigen (TAA) gp100. Furthermore, the modularity of the PLP platform also allows for the rapid synthesis and screening of large libraries of potential side chains, enabling the development of personalized vaccines through the incorporation of patient-specific neoantigens.

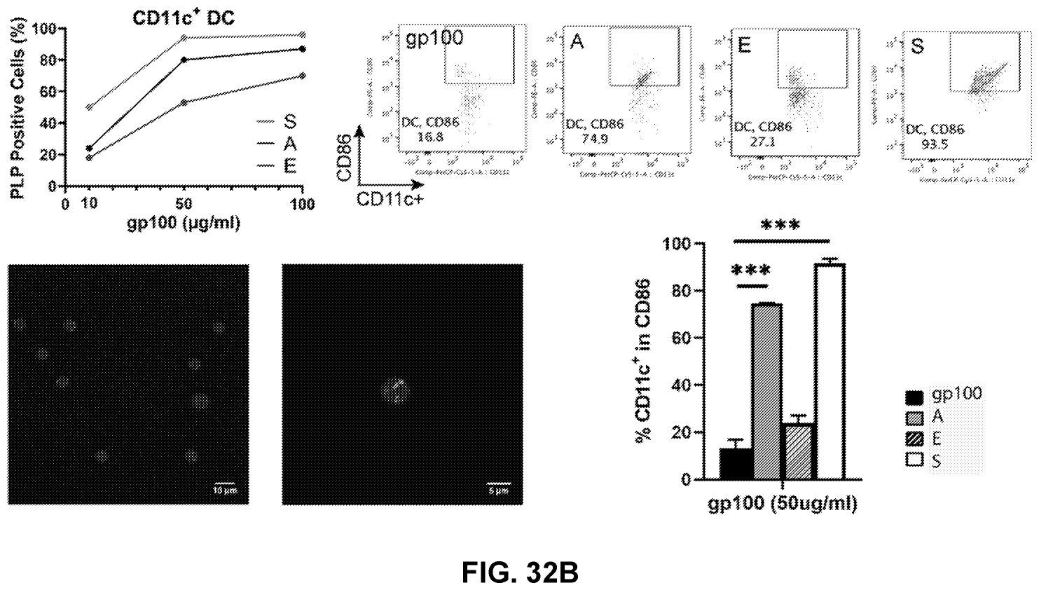

To demonstrate the potential of this delivery platform to enhance immune activation both in vitro and in vivo, we conducted preliminary studies using a melanoma tumor-associated antigen, gp100. Gp100 is a MHC Class I restricted tumor-associated antigen naturally expressed in melanocytes. Studies show that immunization of C57BL/6 mice with gp100 elicits a specific CD8⁺ T cell response and is effective at treating established B16F10 melanoma. Since covalent modification of antigens has the potential to impede T cell activation by altering intracellular antigen processing and presentation on MHC molecules, we tested antigen conjugation to the polymer backbone using various cleavable (disulfide and ester) and non-cleavable (amide) linkers (FIG. 32A). In particular, the intracellularly cleavable disulfide linker, upon endocytosis by antigen presenting cells (APCs), undergoes a disulfide reduction that releases the antigen and can allow endogenous antigen processing and presentation on MHC.

ROMP was used to generate PLPs starting from these monomers. This polymerization is functional group tolerant and allows the direct polymerization of unprotected peptides yielding polymers with controlled molecular weight and low polydispersity. The generalizability of this approach is a unique feature that allows for incorporation of combinations of peptide antigens irrespective of their primary sequence. The direct polymerization can be easily performed by modifying the N-terminus of the peptide sequences with a norbornene moiety by solid phase peptide synthesis (SPPS). Upon cleavage from the resin and purification, the obtained peptide monomers can be polymerized by stirring with the initiator. Using this approach, we first prepared PLPs containing 15 repeating units of gp100 covalently linked to the norbornene backbone using either a non-cleavable amide linkage (A), an unstable ester linkage (E), or an intracellularly cleavable disulfide linkage (S) (FIG. 32A, m=15, n=0). Preliminary in vitro studies using 15 mer PLPs (A, E and S) labeled with a fluorescent dye demonstrated high levels of polymer uptake in mouse splenic CD11c⁺ dendritic cells (DC), after only 30 min incubation (˜95%) in a dose-dependent manner (FIG. 32B). Cellular uptake was further confirmed by confocal microscopy (FIG. 32B). Moreover, DCs that internalized PLPs showed significantly higher expression of surface activation marker CD86⁺ as compared to cells incubated with free gp100 peptide (FIG. 32B).

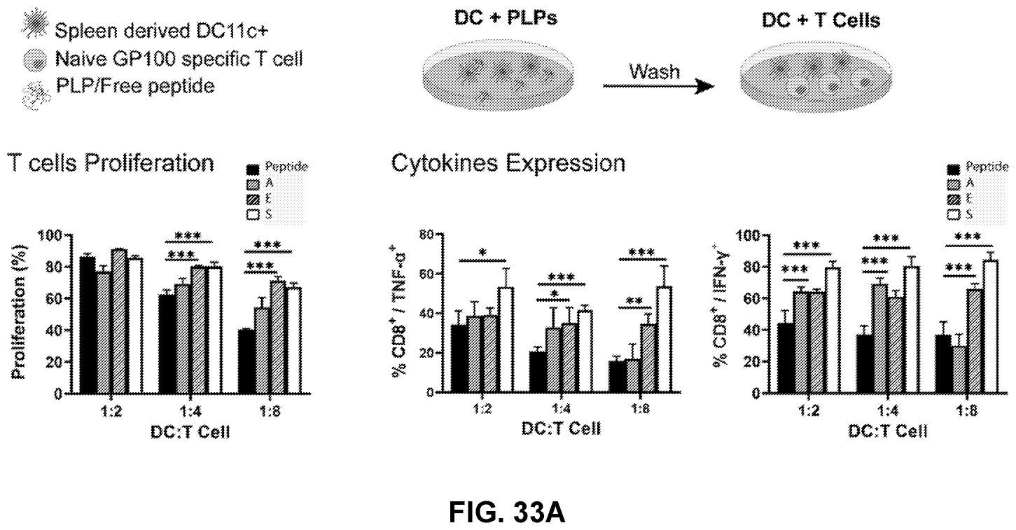

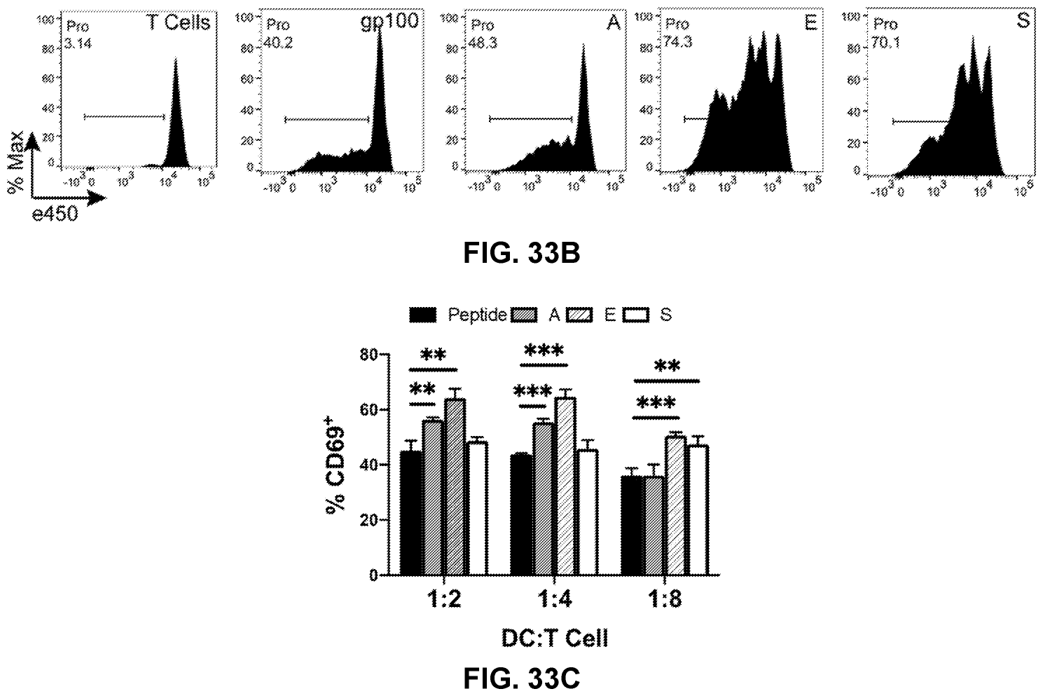

To test the ability of gp100-PLPs to stimulate DC-mediated T cell priming, DCs isolated from wild type mice were incubated with either PLPs or free peptide for 4 hours (FIGS. 33A-33C) and subsequently washed and co-incubated with gp100-specific T cells derived from Pmel mice splenocytes for 4 days. The experiment was performed using different ratios of DC to T cells to screen for the most effective formulation. Increased T cell proliferation (assessed by eFluor 450 dilution) and CD69 expression were observed for PLPs using the disulphide linker (S) compared to other PLPs (A and E linkers) and versus the control peptide (FIGS. 33A-33C), even at high DC dilutions. Additionally, incubation with the disulphide linked PLP led to significantly higher levels of IFN-γ and TNF-α expression in CD8 T cells as compared to PLPs bearing the other linkers (A and E), consistent with uptake data for PLPs by DCs (FIG. 32B).

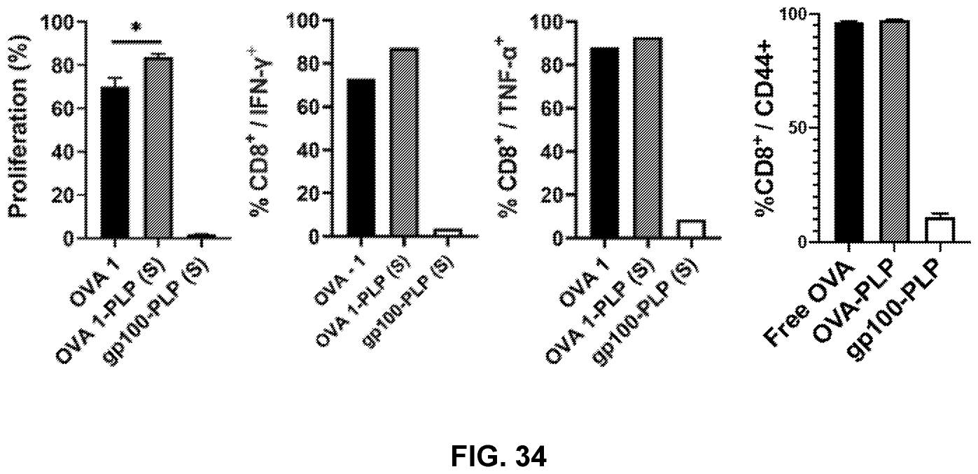

Similar experiments were also performed using the model antigen OVA-1 (SIINFEKL—SEQ ID NO:20) displayed as a PLP (disulphide-linked 15 mer=OVA 1-PLP (S)) to demonstrate the generalizability of the delivery platform to another antigen. In this case, whole splenocytes from OT-1 mice were incubated with OVA-PLPs for 4 days. Again, we found that OVA-PLPs were superior in stimulating OVA-1-specific OT-1 T cell responses compared to free peptides. The control PLPs displaying irrelevant peptides (gp100-PLPs copolymerized with PEG in this case) failed to elicit antigen-specific T cell activity (FIGS. 34A-34B). Taken together, these results indicate that PLPs of peptide antigens are efficiently internalized by DCs and that the use of an intracellularly cleavable disulfide linker allows for higher levels of antigen-specific T cell activation, likely due to enhanced antigen processing and presentation.

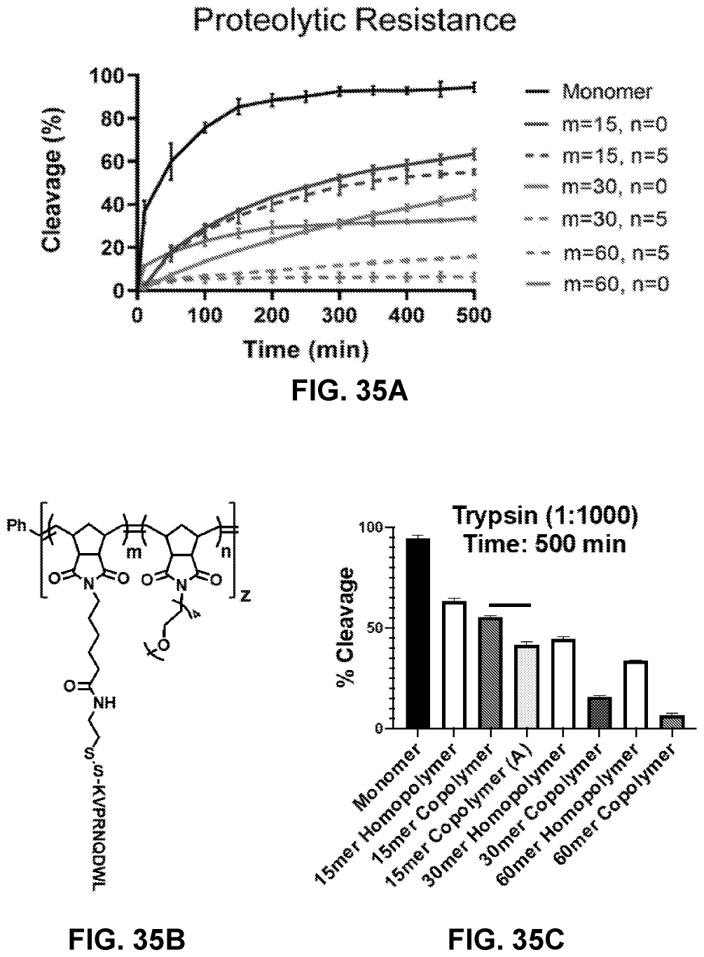

Next, we carefully examined the degree of polymerization (i.e. number of antigens per chain) and the incorporation of polyethylene glycol (PEG) based monomers tuning water solubility and how these parameters affect resistance to proteolytic degradation. All polymers consisting of 15, 30, or 60 repeats (m=15, 30 and 60 in FIG. 32A) of gp100 were synthesized with either 0 or 5 units of PEG (n=0 and n=5 in FIG. 32A). Low levels of in vivo activity can be due to very short in vivo persistence of peptides because of rapid enzymatic degradation. Incubation with the model enzyme trypsin showed a degree of polymerization (m) dependent increase in enzymatic resistance, with higher degrees of polymerization and incorporation of PEG sidechains as the “n” block, having decreased cleavage rates (FIGS. 35A-35C). Without wishing to be bound by any particular theory, it is believed that this is due to increasing PLP globularity and therefore increased enzymatic hindrance. Incorporation of PEG likely leads to further shielding of the PLP side chains.

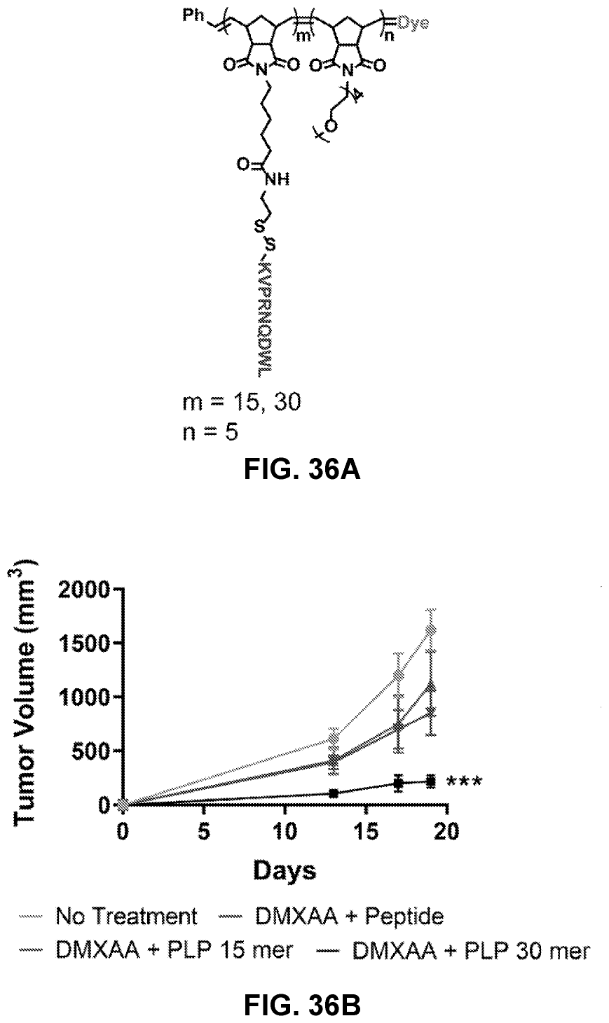

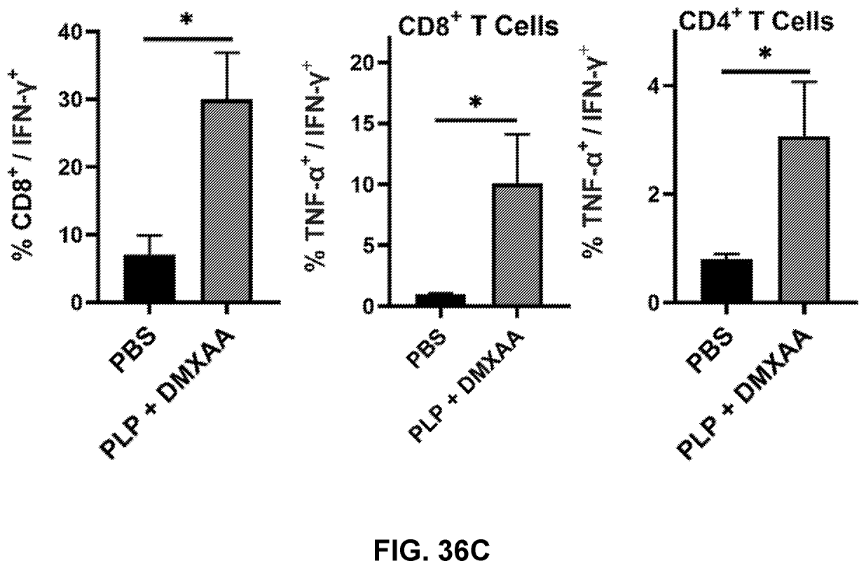

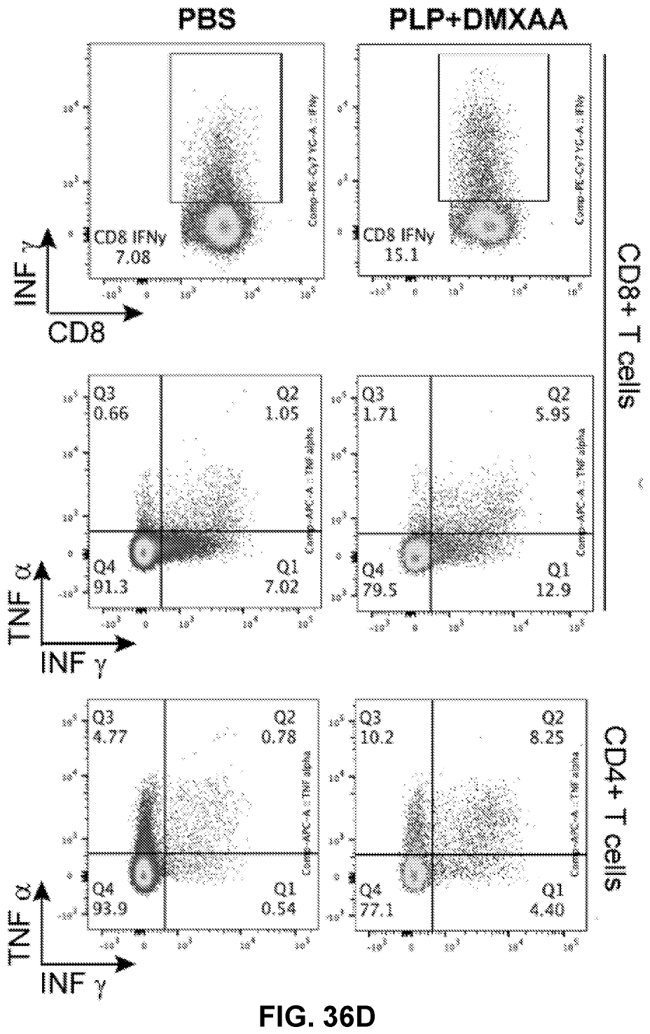

We next investigated the efficacy of gp100-PLPs (disulfide linker) in inducing a robust anti-tumor immune response in vivo. Two different lengths of KVPRNQDWL (SEQ ID NO:10)-based PLPs (m=15 and m=30) were synthesized as statistical copolymers containing 5 units PEG (FIG. 36A). Mice challenged with B16F10 melanoma were treated with either the gp100-PLP or free gp100 peptides in combination with 5,6-dimethylxanthenone-4-acetic acid (DMXAA), a ligand of the Stimulator of Interferon Genes (STING), as an adjuvant. DMXAA was selected for a first in vivo experiment since it has previously demonstrated potent immune activation in mice. Four separate injections were made on days 6, 10, 13, and 17. Although all combinations reduced tumor growth, 30 mer PLP in combination with DMXAA showed the greatest tumor growth inhibition (FIG. 36B). Ex vivo staining of cells isolated from tumor-draining lymph nodes (TDLN) showed increased cytokine expression in both CD4 and CD8 T cells (FIG. 36C). In vivo immune activation was determined by flow cytometry of cells derived from tumor draining lymph nodes (TDLN) of mice treated with either PBS or PLP (30 mer+DMXAA). Representative flow panels are shown in FIG. 36D.

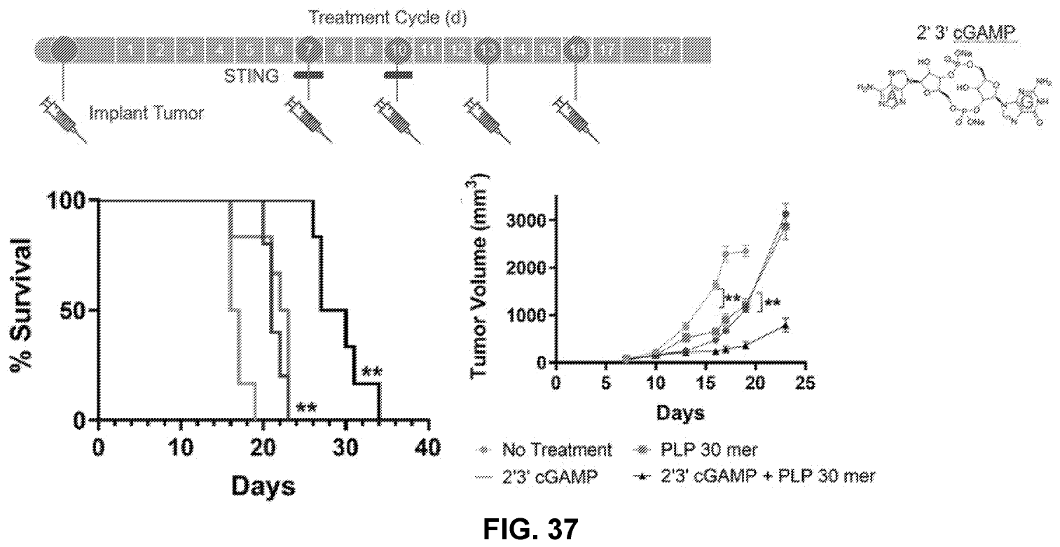

In addition, we tested the effects of combination therapy with the most commonly used STING agonist, 2′3′ cGAMP, which has shown promising results using both human and mouse cell systems. We treated B16F10 tumor-bearing mice with either the gp100-PLP alone or in combination with 2′3′ cGAMP. Four separate injections were made on days 7, 10, 13, and 16. Combination treatment of the 30 mer gp100-PLP (disulphide linked) and 2′3′ cGAMP resulted in significantly reduced tumor burden and increased survival compared to controls (FIG. 37). Interestingly, mice treated with g100-PLP alone (without adjuvant) had similar rates of tumor growth and survival compared to STING agonist controls suggesting that PLPs can act as self-adjuvants. These results demonstrate the potential of formulating peptide antigens as PLPs for efficient internalization by APCs, to induce potent and durable antigen-specific T cell responses owing to their superior proteolytic resistance and self-adjuvanting properties.

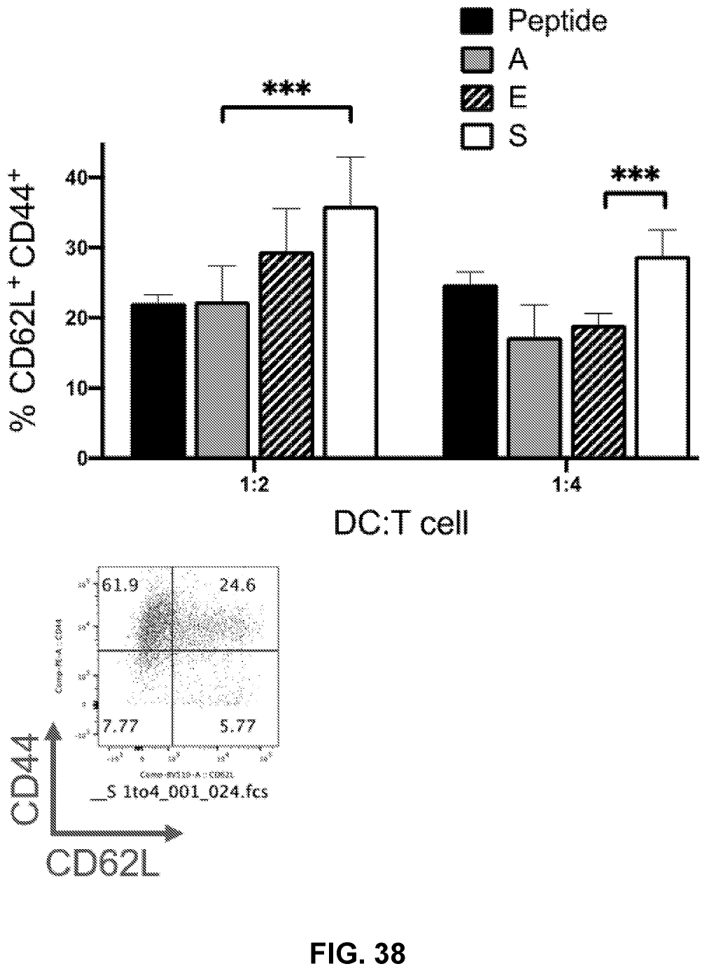

We also compared CD44 and CD62L expression levels of CD3 T cells coincubated with different ratios of DCs previously treated with either free gp100 (Peptide) or gp100-PLPs with antigen linked onto the polymer backbone using different linkage chemistries (A, E, S). Dosages were normalized to amount of antigen 10 ug/ml gp100. ***: p<0.001. CD44 and CD62L expression levels were determined by flow cytometry and the results are shown in FIG. 38. As demonstrated by FIG. 38, PLP treatment leads to expansion of T Cells with a central memory phenotype.

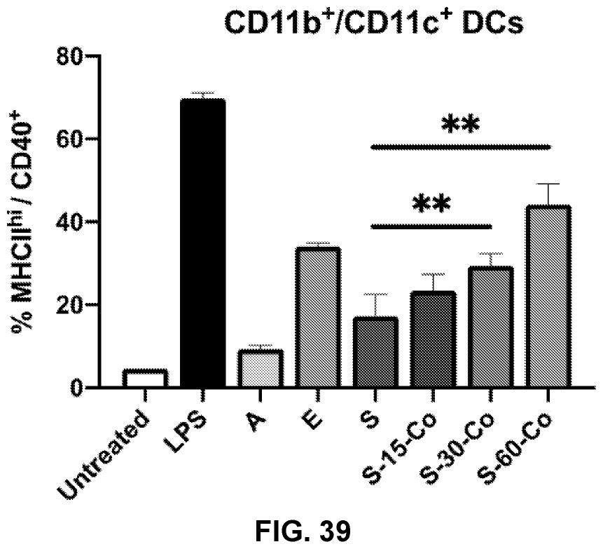

We also compared the level of DC activation using PLPs of different lengths and composition. Isolated DCs were treated overnight with different PLP formulations, using LPS as positive control. The results are shown in FIG. 39. The PLP designations listed in FIG. 39 correspond to the following PLP structures. S-15-Co: disulfide linked PLP, 15mer, copolymerized with 5 monomer units of PEG; S-30-Co: disulfide linked PLP, 30mer, copolymerized with 5 monomer units of PEG; S-60-Co: disulfide linked PLP, 60mer, copolymerized with 5 monomer units of PEG. Dosages were normalized to amount of antigen 50 ug/ml gp100. **: p<0.01. As demonstrated by FIG. 39, DC activation increased as the PLP monomer amount increased.

### Example 5—In Vivo Delivery of Terlipressin PLP in the Management of Blood Pressure

This example demonstrates the positive effect on blood pressure exhibited by the delivery of terlipressin PLP relative to terlipressin peptide alone.

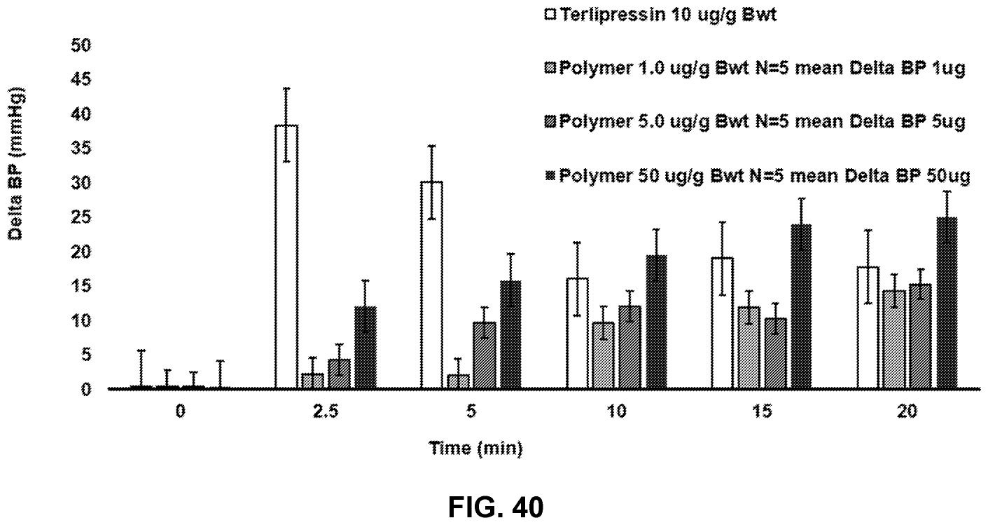

Healthy C57BL/6 mice were injected with either terlipressin peptide or terlipressin PLP and the systolic blood pressure was measured over time by employing a tail-cuff system (CODA™). The mice were anesthetized with ketamine and placed in restraints over a heated pad. Following 5 minutes of background measurements, the drugs were administrated by intraperitoneal injection (IP) and the measurements continued for another 20 minutes (limited by the effect of ketamine). The delta BP was measured and plotted as a function of time. The results are shown in FIG. 40.

While terlipressin peptide alone caused an immediate and very large increase in blood pressure, which dropped rapidly in a manner of few minutes, the terlipressin PLP generated a sustained and more stable effect. Dose dependency was observed for increasing dosages of the terlipressin PLP, suggesting the therapeutic effect can be tuned.

### Statements Regarding Incorporation by Reference and Variations

(1) All references cited throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).
(2) Pending U.S. Provisional Patent Application No. 62/885,655 filed Aug. 12, 2019 is hereby incorporated by reference.
(3) The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.
(4) When a group of substituents is disclosed herein, it is understood that all individual members of that group and all subgroups are disclosed separately. When a Markush group or other grouping is used herein, all individual members of the group and all combinations and subcombinations possible of the group are intended to be individually included in the disclosure. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.
(5) It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”
(6) Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. Nothing herein is to be construed as an admission that the invention is not entitled to antedate such disclosure by virtue of prior invention.
(7) Whenever a range is given in the specification, for example, a temperature range, a time range, or a composition or concentration range, all intermediate ranges and subranges, as well as all individual values included in the ranges given are intended to be included in the disclosure. As used herein, ranges specifically include the values provided as endpoint values of the range. For example, a range of 1 to 100 specifically includes the end point values of 1 and 100. It will be understood that any subranges or individual values in a range or subrange that are included in the description herein can be excluded from the claims herein.
(8) As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.
(9) One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.
(10) All references, including publications, patent applications, and patents, cited herein are hereby incorporated by reference to the same extent as if each reference were individually and specifically indicated to be incorporated by reference and were set forth in its entirety herein.
(11) The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.
(12) Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A polymer characterized by the formula (FX2a), (FX2b), or (FX2c): 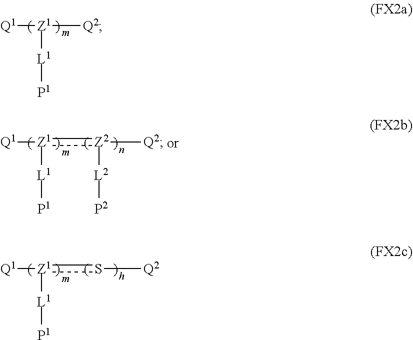
wherein
each Z¹ is independently a first polymer backbone group and each Z² is independently a second polymer backbone group;
each S is independently a repeating unit having a composition different from the first repeating unit;
Q¹ is a first backbone terminating group and Q² is a second backbone terminating group;
each L¹ is independently a first linking group, each L² is independently a second linking group;
each P¹ is the polymer side chain comprising the peptide; wherein each P² is a polymer side chain having a composition different from that of P¹;
each m is independently an integer selected from the range of 2 to 100;
each n is independently an integer selected from the range of 0 to 100; and
each h is independently an integer selected from the range of 0 to 100,
provided that each of the first polymer backbone group and/or the second polymer backbone group is a polymerized norbornene dicarboxyimide monomer, and
wherein the polymer fulfills at least one of the following properties:
(iv) the polymer has a degree of polymerization of 5 to 100,
(v) the peptide comprises 5 to 100 amino acids, and
(vi) the polymer has a peptide density of greater than or equal to 50%, as defined by the following formula:
$$\frac{{P}^{1}}{{P}^{1} + {P}^{2} + S} \times 100.$$

2. The polymer of claim 1, wherein the polymer fulfills at least one of the following properties (i)-(iii).

3. The polymer of claim 1 or 2, wherein the polymer has a degree of polymerization of 5 to 50.

4. The polymer of any one of claims 1-3, wherein the peptide comprises 8 to 60 amino acids.

5. The polymer of any one of claims 1-4, wherein the polymer has a peptide density of greater than or equal to 70%.

6. The polymer of any one of claims 1-5, wherein the polymer has a peptide density of greater than or equal to 90%.

7. The polymer of any one of claims 1-6, wherein each Z¹ connected to L¹, and P¹ or a combination thereof is independently characterized by the formula (FX3a):

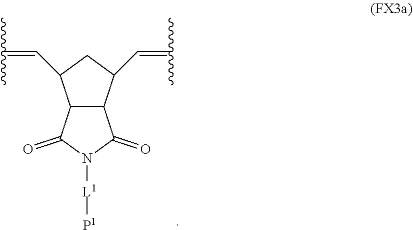

8. The polymer of any one of claims 1-7, wherein each Z² connected to L², and P² or a combination thereof is independently characterized by the formula (FX4a):

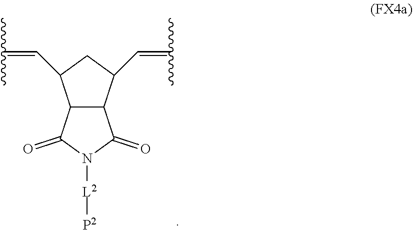

9. The polymer of any one of claims 1-8, wherein each of Q¹ and Q² is independently selected from a hydrogen, C₁-C₃₀ alkyl, C₃-C₃₀ cycloalkyl, C₅-Cao aryl, C₅-C₃₀ heteroaryl, C₁-Cao acyl, C₁-C₃₀ hydroxyl, C₁-C₃₀ alkoxy, C₂-C₃₀ alkenyl, C₂-C₃₀ alkynyl, C₅-C₃₀ alkylaryl, —CO₂R³, —CONR⁴R⁵, —COR⁶, —SOR⁷, —OSR⁸, —SO₂R⁹, —OR¹⁰, —SR¹¹, —NR¹²R¹³, —NR¹⁴COR¹⁵, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R³-R¹⁵ is independently H, C₅-C₁₀ aryl or C₁-C₁₀ alkyl.

10. The polymer of any one of claims 1-9, wherein each of L¹ and L² is independently selected from a single bond, an oxygen, and groups having an alkyl group, an alkenylene group, an arylene group, an alkoxy group, an acyl group, a triazole group, a diazole group, a pyrazole group, and combinations thereof.

11. The polymer of claim 10, wherein each of L¹ and L² is independently selected from a single bond, —O—, C₁-C₁₀ alkyl, C₂-C₁₀ alkenylene, C₃-C₁₀ arylene, alkoxy, C₁-C₁₀ acyl and combinations thereof.

12. The polymer of any one of claims 1-11, wherein P¹ is a terlipressin peptide, an anti-VEGF peptide, an ABT-898 peptide, a neoantigen-Melanoma peptide, an antigen-M30 peptide, an antigen-gp100 Melanoma peptide, or a derivative, a variant, an analogue, or a fragment of terlipressin peptide, an anti-VEGF peptide, an ABT-898 peptide, a neoantigen-melanoma peptide, an antigen-M30 peptide, or an antigen-gp100 Melanoma peptide.

13. The polymer of any one of claims 1-12, wherein the polymer has a weight average molecular weight of from 1 kDa to 1,000 kDa.

14. The polymer of any one of claims 1-13, wherein the polymer has a weight average molecular weight of from 1 kDa to 100 kDa.

15. The polymer of any one of claims 1-14, wherein the polymer has a weight average molecular weight of from 5 kDa to 50 kDa.

16. A polymer comprising:
a first polymer block comprising at least 2 first repeating units; wherein each of the first repeating units of the first polymer block comprises a first polymer backbone group directly or indirectly covalently linked to a first polymer side chain group comprising a peptide;
wherein the peptide is a terlipressin peptide or a derivative, analogue, variant, isomer or fragment of a terlipressin peptide; and
wherein the polymer exhibits efficacy for treatment or management of a condition selected from the group consisting of hepatorenal syndrome, low blood pressure, bleeding esophageal varices, septic shock and paracentesis-induced circulatory dysfunction.

17. The polymer of claim 16, wherein the peptide comprises a sequence having 80% or greater sequence homology of SEQ ID NO: 1 (GGGCYFQNCPKG).

18. The polymer of claim 16, wherein the peptide comprises the amino acid sequence of SEQ ID NO: 1 (GGGCYFQNCPKG).

19. The polymer of claim 16, wherein the peptide is a derivative, analogue, variant, isomer or fragment of said terlipressin peptide.

20. A polymer comprising:
a first polymer block comprising at least 2 first repeating units; wherein each of the first repeating units of the first polymer block comprises a first polymer backbone group directly or indirectly covalently linked to a first polymer side chain group comprising a peptide;
wherein the peptide is an anti-VEGF peptide, an ABT-898 peptide or a derivative, analogue, variant, isomer or fragment of an anti-VEGF peptide or an ABT-898 peptide; and
wherein the polymer exhibits efficacy for treatment or management of age related macular degeneration.

21. The polymer of claim 20, wherein the peptide comprises a sequence having 80% or greater sequence homology of SEQ ID NO: 3 (PCAIWF) or SEQ ID NO: 4 (GVi(allo)SQIRP).

22. The polymer of claim 20, wherein the peptide comprises the amino acid sequence of SEQ ID NO: 3 (PCAIWF) or SEQ ID NO: 4 (GVi(allo)SQIRP).

23. The polymer of claim 20, wherein the peptide is a derivative, analogue, variant, isomer or fragment of said anti-VEGF peptide or said ABT-898 peptide.

24. A polymer comprising:
a first polymer block comprising at least 2 first repeating units; wherein each of the first repeating units of the first polymer block comprises a first polymer backbone group directly or indirectly covalently linked to a first polymer side chain group comprising a peptide;
wherein the peptide is a neoantigen-melanoma peptide or a derivative, analogue, variant, isomer or fragment of a neoantigen-melanoma peptide; and
wherein the polymer exhibits efficacy for treatment or management of cancer.

25. The polymer of claim 24, wherein the peptide comprises a sequence having 80% or greater sequence homology of SEQ ID NO: 5 (SHCHWNDLAVIPAGVVHNWDFEPRKVS), SEQ ID NO: 6. (GRGHLLGRLAAIVGKQVLLGRKVVVVR), SEQ ID NO: 7. (SKPSFQEFVDWENVSPELNSTDQPFL), or SEQ ID NO: 8 (REGVELCPGNKYEMRRHGTTHSLVIHD).

26. The polymer of claim 24, wherein the peptide comprises the amino acid sequence of SEQ ID NO: 5 (SHCHWNDLAVIPAGVVHNWDFEPRKVS), SEQ ID NO: 6. (GRGHLLGRLAAIVGKQVLLGRKVVVVR), SEQ ID NO: 7. (SKPSFQEFVDWENVSPELNSTDQPFL), or SEQ ID NO: 8 (REGVELCPGNKYEMRRHGTTHSLVIHD).

27. The polymer of claim 24, wherein the peptide is a derivative, analogue, variant, isomer or fragment of said neoantigen—melanoma peptide.

28. A polymer comprising:
a first polymer block comprising at least 2 first repeating units; wherein each of the first repeating units of the first polymer block comprises a first polymer backbone group directly or indirectly covalently linked to a first polymer side chain group comprising a peptide;
wherein the peptide is an antigen-M30 peptide, an antigen-gp100 melanoma peptide or a derivative, analogue, variant, isomer or fragment of an antigen-M30 peptide or an antigen-gp100 melanoma peptide; and
wherein the polymer exhibits efficacy for treatment or management of cancer.

29. The polymer of claim 28, wherein the peptide comprises a sequence having 80% or greater sequence homology of SEQ ID NO: 9 (VDWENVSPELNSTDQ) or SEQ ID NO: 10 (KVPRNQDWL).

30. The polymer of claim 28, wherein the peptide comprises the amino acid sequence of SEQ ID NO: 9 (VDWENVSPELNSTDQ) or SEQ ID NO: 10 (KVPRNQDWL).

31. The polymer of claim 28, wherein the peptide is a derivative, analogue, variant, isomer or fragment of said antigen-M30 peptide or said antigen-gp100 melanoma peptide.

32. The polymer of any of the preceding claims comprising a homopolymer.

33. The polymer of any of the preceding claims comprising a copolymer.

34. The polymer of any of the preceding claims comprising a brush polymer.

35. The polymer of any of the preceding claims comprising a brush polymer.

36. The polymer of any of the preceding claims comprising a high-density brush polymer characterized by a brush density greater than or equal to 70%.

37. The polymer of any of the preceding claims comprising a brush block copolymer.

38. The polymer of any of the preceding claims, wherein the first polymer block comprises at least 5 first repeating units

39. The polymer of any of the preceding claims, wherein the first polymer block comprises 5-30 first repeating units.

40. The polymer of any one of claims 16-39, wherein the polymer is characterized by a degree of polymerization of 2 to 1000.

41. The polymer of any of the preceding claims, wherein the polymer is characterized by a polydispersity index less than 1.75.

42. The polymer of any of the preceding claims, wherein the peptide is a branched polypeptide, a linear polypeptide or a cross-linked polypeptide.

43. The polymer of any of the preceding claims, wherein at least a portion of the peptide is linked to the polymer backbone group via an enzymatically degradable linker.

44. The polymer of claim 41, wherein the enzymatically degradable linker is a matrix metalloproteinase (MMP) cleavage sequence, cathepsin B cleavage sequence, ester bond, reductive sensitive bond—disulfide bond, pH sensitive bond—imine bond or any combinations of these.

45. The polymer of any of the preceding claims, wherein at least a portion of the peptide side-chain is linked to the polymer backbone or consists of a degradable or triggerable linker.

46. The polymer of any one of claim 16-45 characterized by the formula (FX1a), (FX1b), (FX1c), (FX1d); (FX1e); (FX1f); or (FX1g):
Q¹-T-Q² (FX1a);
Q¹-T-[S]ₕ-Q² (FX1b);
Q¹-[S]ₕ-T-Q² (FX1c);
Q¹-[S]ᵢ-T-[S]ₕ-Q² (FX1d);
Q¹-[S]ᵢ-T-[S]ₕ-T-Q² (FX1e);
Q¹-T-[S]ᵢ-T-[S]ₕ-Q² (FX1f); or
Q¹-T-[S]ᵢ-T-[S]ₕ-T-Q² (FX1g);
wherein each T is independently the first polymer block comprising the first repeating units and each S is independently an additional polymer block; Q¹ is a first polymer block terminating group; Q² is a second polymer block terminating group; and
wherein h is zero or an integer selected over the range of 1 to 1000 and i is zero or an integer selected over the range of 1 to 1000.

47. The polymer of claim 46, wherein each -T- is independently -[Y¹]ₘ-; wherein each Y¹ is independently the first repeating unit of the first polymer block; and each m is independently an integer selected from the range 0 to 1000, provided that at least one m is an integer selected from the range 1 to 1000.

48. The polymer of any one of claim 16-45 characterized by the formula (FX2a), (FX2b), or (FX2c): 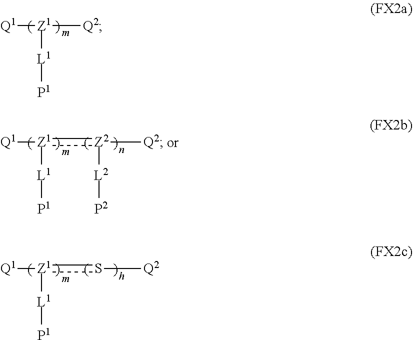
wherein
each Z¹ is independently a first polymer backbone group and each Z² is independently a second polymer backbone group;
each S is independently a repeating unit having a composition different from the first repeating unit;
Q¹ is a first backbone terminating group and Q² is a second backbone terminating group;
each L¹ is independently a first linking group, each L² is independently a second linking group;
each P¹ is the polymer side chain comprising the peptide; wherein each P² is a polymer side chain having a composition different from that of P¹;
each m is independently an integer selected from the range of 2 to 1000;
each n is independently an integer selected from the range of 0 to 1000; and
each h is independently an integer selected from the range of 0 to 1000.

49. The polymer of any one of claims 16-48, wherein each of the first polymer backbone group and/or the second polymer backbone group is a polymerized norbornene dicarboxyimide monomer.

50. The polymer of any one of claims 16-49, wherein each polymer backbone group of the polymer is a polymerized norbornene dicarboxyimide monomer.

51. The polymer of claim 48, wherein each of Z¹ and Z² is independently a substituted or unsubstituted norbornene, oxanorbornene, olefin, cyclic olefin, cyclooctene, or cyclopentadiene.

52. The polymer of claim 48, wherein each Z¹ connected to L¹, and P¹ or a combination thereof is independently characterized by the formula (FX3a) or (FX3b): 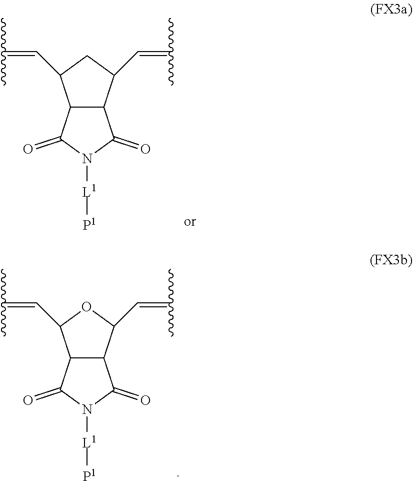
and wherein each Z² connected to L², and P² or a combination thereof is independently characterized by the formula (FX4a) or (FX4b)

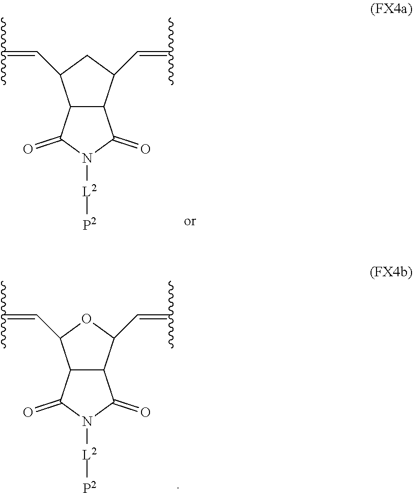

53. The polymer of claim 48, wherein each Z¹ connected to L¹, and P¹ or a combination thereof is independently characterized by the formula (FX3a):

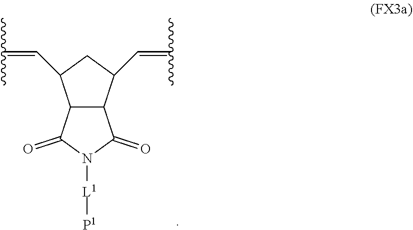

54. The polymer of claim 48 or claim 53, wherein each Z² connected to L², and P² or a combination thereof is independently characterized by the formula (FX4a):

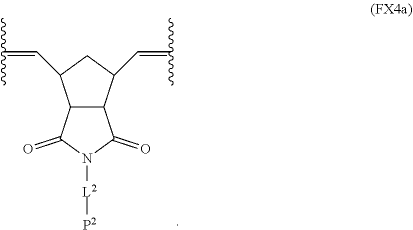

55. The polymer of any one of claims 46-54, wherein each of Q¹ and Q² is independently selected from a hydrogen, C₁-C₃₀ alkyl, C₃-C₃₀ cycloalkyl, C₅-Cao aryl, C₅-C₃₀ heteroaryl, C₁-Cao acyl, C₁-C₃₀ hydroxyl, C₁-C₃₀ alkoxy, C₂-C₃₀ alkenyl, C₂-C₃₀ alkynyl, C₅-C₃₀ alkylaryl, —CO₂R³, —CONR⁴R⁵, —CORE, —SORT, —OSR⁸, —SO₂R⁹, —OR¹⁰, —SR¹¹, —NR¹²R¹³, —NR¹⁴COR¹⁵, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R³-R¹⁵ is independently H, C₅-C₁₀ aryl or C₁-C₁₀ alkyl.

56. The polymer of any one of claims 48-55, wherein each of L¹ and L² is independently selected from a single bond, an oxygen, and groups having an alkyl group, an alkenylene group, an arylene group, an alkoxy group, an acyl group, a triazole group, a diazole group, a pyrazole group, and combinations thereof.

57. The polymer of claim 56, wherein each of L¹ and L² is independently selected from a single bond, —O—, C₁-C₁₀ alkyl, C₂-C₁₀ alkenylene, C₃-C₁₀ arylene, alkoxy, C₁-C₁₀ acyl and combinations thereof.

58. The polymer of any one of claims 46-57, wherein P¹ or P² is a terlipressin peptide, an anti-VEGF peptide, an ABT-898 peptide, a neoantigen—Melanoma peptide, an antigen-M30 peptide, an antigen-gp100 Melanoma peptide, or a derivative, a variant, an analogue, or a fragment of terlipressin peptide, an anti-VEGF peptide, an ABT-898 peptide, a neoantigen—melanoma peptide, an antigen-M30 peptide, or an antigen-gp100 Melanoma peptide.

59. The polymer of any one of claims 46-57, wherein P¹ or P² is the peptide comprising a sequence having 80% or greater sequence homology of SEQ ID NO: 1 (GGGCYFQNCPKG), SEQ ID NO: 3 (PCAIWF), SEQ ID NO: 4 (GVi(allo)SQIRP), SEQ ID NO: 5 (SHCHWNDLAVIPAGVVHNWDFEPRKVS), SEQ ID NO: 6. (GRGHLLGRLAAIVGKQVLLGRKVVVVR), SEQ ID NO: 7. (SKPSFQEFVDWENVSPELNSTDQPFL), or SEQ ID NO: 8 (REGVELCPGNKYEMRRHGTTHSLVIHD), SEQ ID NO: 9 (VDWENVSPELNSTDQ) or SEQ ID NO: 10 (KVPRNQDWL), SEQ ID NO: 11 (CKGKGAKCSRLMYDCCTGSCRSGKC), SEQ ID NO: 12. (ADNKCNSLRREIACGQCRDKVKTDGYFYECCTSDSTFKKCQDLLH), SEQ ID NO: 13. (EESMLLSCPDLSCPTGYTCDVLTKKCKRLSDELWDH), SEQ ID NO: 14 (GCCSDPRCRYRCR) or SEQ ID NO: 15. (PVNFKFLSH), or SEQ ID NO: 16 (AKPSY-Hyp-Hyp-T-DOPA-K).

60. The polymer of any one of claims 46-57, wherein P¹ or P² is the peptide comprising the amino acid sequence of SEQ ID NO: 1 (GGGCYFQNCPKG), SEQ ID NO: 3 (PCAIWF), SEQ ID NO: 4 (GVi(allo)SQIRP), SEQ ID NO: 5 (SHCHWNDLAVIPAGVVHNWDFEPRKVS), SEQ ID NO: 6. (GRGHLLGRLAAIVGKQVLLGRKVVVVR), SEQ ID NO: 7. (SKPSFQEFVDWENVSPELNSTDQPFL), or SEQ ID NO: 8 (REGVELCPGNKYEMRRHGTTHSLVIHD), SEQ ID NO: 9 (VDWENVSPELNSTDQ) or SEQ ID NO: 10 (KVPRNQDWL), SEQ ID NO: 11 (CKGKGAKCSRLMYDCCTGSCRSGKC), SEQ ID NO: 12. (ADNKCNSLRREIACGQCRDKVKTDGYFYECCTSDSTFKKCQDLLH), SEQ ID NO: 13. (EESMLLSCPDLSCPTGYTCDVLTKKCKRLSDELWDH), SEQ ID NO: 14 (GCCSDPRCRYRCR) or SEQ ID NO: 15. (PVNFKFLSH), or SEQ ID NO: 16 (AKPSY-Hyp-Hyp-T-DOPA-K).

61. The polymer of any one of the preceding claims, wherein the polymer is stable against enzymatic digestion.

62. The polymer of any one of the preceding claims, wherein the polymer is stable against enzymatic digestion by a metalloproteinase.

63. The polymer of any one of the preceding claims, wherein the polymer is stable against enzymatic digestion by matrix metalloproteinases and thermolysin.

64. The polymer of any one of the preceding claims, wherein the polymer is stable against enzymatic digestion for at least 450 minutes.

65. The polymer of any one of the preceding claims, wherein the polymer is stable against enzymatic digestion by thermolysin such that less than 20% of thermolysin-cleavable sites are cleaved by thermolysin after at least 450 minutes of the polymer's exposure to thermolysin.

66. The polymer of any one of the preceding claims, wherein each polymer individually solvated by water when a plurality of said polymers is dispersed in water.

67. A method of treatment comprising administering to a subject an effective amount of the polymer of any one of claims 1-66.

68. A method of treating or managing a condition in a subject comprising:
administering to a subject an effective amount of a polymer comprising:
a first polymer block comprising at least 2 first repeating units; wherein each of the first repeating units of the first polymer block comprises a first polymer backbone group directly or indirectly covalently linked to a first polymer side chain group comprising a peptide;
wherein the peptide is a terlipressin peptide or a derivative, analogue, variant, isomer or fragment of an terlipressin peptide;
wherein the condition is selected from the group consisting of hepatorenal syndrome, low blood pressure, bleeding esophageal varices, septic shock and paracentesis-induced circulatory dysfunction.

69. A method of treating or managing a condition in a subject comprising:
administering to a subject an effective amount of a polymer comprising:
a first polymer block comprising at least 2 first repeating units; wherein each of the first repeating units of the first polymer block comprises a first polymer backbone group directly or indirectly covalently linked to a first polymer side chain group comprising a peptide;
wherein the peptide is an anti-VEGF peptide, an ABT-898 peptide or a derivative, analogue, variant, isomer or fragment of an anti-VEGF peptide or an ABT-898 peptide;
wherein the condition is age related macular degeneration.

70. A method of treating or managing a condition in a subject comprising:
administering to a subject an effective amount of a polymer comprising:
a first polymer block comprising at least 2 first repeating units; wherein each of the first repeating units of the first polymer block comprises a first polymer backbone group directly or indirectly covalently linked to a first polymer side chain group comprising a peptide;
wherein the peptide is a neoantigen-melanoma peptide or a derivative, analogue, variant, isomer or fragment thereof;
wherein the condition is cancer.

71. A method of treating or managing a condition in a subject comprising:
administering to a subject an effective amount of a polymer comprising:
a first polymer block comprising at least 2 first repeating units; wherein each of the first repeating units of the first polymer block comprises a first polymer backbone group directly or indirectly covalently linked to a first polymer side chain group comprising a peptide;
wherein the peptide is an antigen-M30 peptide, an antigen-gp100 melanoma peptide or a derivative, analogue, variant, isomer or fragment of an antigen-M30 peptide or an antigen-gp100 melanoma peptide;
wherein the condition is cancer.

72. The method of any of claims 67-71, further comprising contacting a target tissue of the subject with the polymer or a metabolite or product thereof.

73. The method of any of claims 67-72, further comprising contacting a target cell of the subject with the polymer or a metabolite or product thereof.

74. The method of any of claims 67-73, further comprising contacting a target receptor of the subject with the polymer or a metabolite or product thereof.
